(19) United States
(12) Patent Application Publication
(10) Pub. No.: US 2002/0045591 A1
(43) Pub. Date: Apr. 18, 2002

(54) Methods and therapeutic compositions for treating cancer

(76) Inventors: Benjamin Geiger, Rehovot (IL); Avri Ben-Ze'ev, Rehovot (IL); Einat Sadot, Moshav Sitriya (IL)

Correspondence Address: SOL SHEINBEIN, c/o ANTHONY CASTORINA, SUITE 207, 2001 JEFFERSON DAVIS HIGHWAY, ARLINGTON, VA, 22202, US

(21) Appl. No.: 09/905,983

(22) Filed: Jul. 17, 2001

Related U.S. Application Data
(62) Division of application No. 09/318,633, filed on May 26, 1999, now abandoned.

(30) Foreign Application Priority Data
May 26, 1998 (IL) 124650

(51) Int. Cl. A61K 48/00

(52) U.S. Cl. 514/44

(57) ABSTRACT

Methods and therapeutic compositions for the treatment of cancer are disclosed. Specifically, peptides including β-catenin binding domains and polynucleotide sequences encoding same, such as cadherins and o-catenins and polynucleotide sequences encoding same, effective in methods and compositions for treating cancers associated with abnormally high levels of β-catenin, such as colon cancers and melanomas.

This is a divisional of U.S. patent application Ser. No. 09/318,633, filed May 26, 1999.

### FIELD AND BACKGROUND OF THE INVENTION

The present invention relates to methods and therapeutic compositions for the treatment of cancer and, more particularly, to peptides including β-catenin binding domains, such as cadherins and o-catenins, and to polynucleotide sequences encoding same, therapeutically effective methods and compositions for treating cancers associated with abnormally high levels of β-catenin, such as, but not limited to, colon cancers (carcinomas) and melanomas.

Cell adhesion and involvement of adhesion-related proteins in transmembrane signaling are the object of major studies in modern cell biology. The major advances in this field, and in particular in the involvement of β-catenin in cell adhesion and signaling, are summarized in the following reviews:

Bullions L. C. and Levine J. A. (1998) The role of beta-catenin in cell adhesion, signal transduction, and cancer. Current Opinion in Oncology, 10:81-87.

Brown D. J. and Moon T. R. (1998) Wnt signaling: why is everything so negative? Current Opinion in Cell Biology, 10:182-187.

Willert K. and Nusse R. (1998) β-catenin: a key mediator of Wnt signaling. Current Opinion in Genetics & Development, 8:95-102.

Ben-Ze'ev A. and Geiger B. (1998) Differential molecular interaction of β-catenin and plakoglobin in adhesion, signaling and cancer. Current Opinion in Cell Biology, 10:629-639.

Polakis P. (1999) The oncogenic activation of β-catenin. Current Opinion in Genetics & Development, 9:15-21.

Cox T. R and Peifer M. Wingless signaling: The inconvenient of complexities of life. Current Biology, 9:R140-R144.

Ben-Ze'ev A. (1997) Cytoskeletal and adhesion proteins as tumor suppressors. Current Opinion in Cell Biology, 9:99-108.

Cell-cell adhesion plays an important role in tissue morphogenesis and homeostasis, and is commonly mediated by cadherins, a family of Ca²⁺-dependent transmembrane adhesion receptors. Cadherins were shown to form homophilic interactions with similar receptors on neighboring cells, while their cytoplasmic domains interact with the cytoskeleton. The latter interactions are essential for stable adhesion and are mediated via β-catenin, or its closely related homolog γ-catenin (plakoglobin), which interact with microfilaments through α-catenin and α-actinin.

In addition, β-catenin can translocate into the nucleus, where it is involved together with transcription factors of the LEF/TCF family in the transcription of specific genes.

The β-Catenin/plakoglobin homologue in Drosophila, armadillo, was shown to be involved in the wingless (wg) signaling pathway that regulates cell fate during development. In Xenopus, β-catenin participates in the wnt-signaling pathway that determines body axis formation and overexpression of β-catenin and plakoglobin in Xenopus embryos was shown to induce double axis formation. In both Drosophila and Xenopus it was demonstrated that the signaling activity of armadillo/β-catenin is independent of cadherin-based adhesion.

On the other hand, this signaling is strongly affected by the levels of cadherin expression since overexpression of cadherin mimics the wg phenotype in Drosophila and blocks β-catenin signaling in Xenopus, suggesting that cadherin may be a negative regulator of armadillo/β-catenin signaling.

In cultured cells, wnt overexpression elicits adhesion-related responses and increased levels of β-catenin and plakoglobin. β-Catenin levels are regulated by glycogen synthase kinase-3β (GSK-3) and adenomatous polyposis coli (APC) tumor suppressor protein which are thought to target β-catenin for degradation by the ubiquitin-proteasome system. When β-catenin levels are high, it can associate with architectural transcription factors of the lymphoid enhancing binding factor/T-cell factor (LEF/TCF) family and translocate into the nucleus. In the nucleus, the β-catenin-LEF/TCF complex activates transcription of LEF/TCF-responsive genes that are not yet known in mammalian cells, but have partially been characterized in Xenopus and Drosophila.

Elevation of β-catenin in colon carcinoma cells that express a mutant APC molecule, or in melanoma where mutations in the NH₂-terminal domain of β-catenin were detected (both inhibiting P-catenin degradation), is oncogenic most probably due to constitutive activation of target genes which contributes to tumor progression. Interestingly, plakoglobin was shown to suppress tumorigenicity when overexpressed in various cells, and displays loss of heterozigosity in sporadic ovarian and breast carcinoma. Moreover, upon induction of plakoglobin expression in human fibrosarcoma and SV40-transformed 3T3 cells β-catenin is displaced from its complex with cadherin and directed to degradation.

Thus, β-catenin-mediated signaling can also be influenced by the tumor suppressor molecule adenomatous polyposis coli (APC). Both plakoglobin and β-catenin can independently associate with APC and further interact with glycogen synthase kinase 3β (GSK-3β), the homologue of zw3 in Drosophila. Phosphorylation of β-catenin by the APC-GSK-3β complex leads to its degradation by the ubiquitin-proteasome system. Failure of this degradation system in cells expressing mutant APC or β-catenin leads to the accumulation of β-catenin and is common in human colon cancer and melanoma. In addition, in azoxymethane-induced rat colon tumors and in certain human colon cancers expressing mutant APC, β-catenin was shown to accumulate in the cytoplasm and in the nuclei of the tumor cells. Interestingly, apart from the increase in β-catenin levels in certain tumors, a reduction in E-cadherin levels was also found in many carcinomas, and the invasiveness of these tumor cells could be suppressed by overexpression of E-cadherin. Moreover, transfection of E-cadherin into certain human colon carcinoma cells resulted in increased cell-substratum adhesion and decreased cell growth and gelatinase secretion, suggesting a tumor suppressive role for E-cadherin.

While reducing the present invention to practice the mechanisms underlying nuclear accumulation of β-catenin and/or plakoglobin were characterized and some of the partners associated with both proteins in the nucleus identified. Furthermore, the nuclear translocation and transactivation abilities of wt and mutant β-catenin and plakoglobin constructs were compared and it was found that these two proteins differ considerably in these properties, demonstrating that N-cadherin, as well as α-catenin can drive β-catenin from the nucleus to the cytoplasm and consequently block activation of LEF-1-responsive transcription.

Furthermore, the ability of the cytoplasmic domains of N- and E-cadherin to modulate β-catenin localization, stability and transactivation potential was characterized. It is shown that expression of the cytoplasmic tail of cadherin, either membrane bound or soluble, protects endogenous β-catenin from degradation and blocks its transactivation capability. In colon cancer cells containing mutant APC (and hence high levels of β-catenin) expression of the various cadherin derivatives, especially its soluble cytoplasmic tail, strongly suppressed β-catenin-mediated transactivation.

We conclude that the deregulated transactivation associated with elevated β-catenin in certain tumors can be suppressed by cadherins and α-catenins which are known to include β-catenin binding domains.

### SUMMARY OF THE INVENTION

According to an aspect of the present invention there is provided a polynucleotide comprising a nucleotide sequence encoding a cytoplasmic portion of cadherin.

According to still further features in the described preferred embodiments the nucleotide sequence includes a portion of a SEQ ID NO. selected from the group consisting of SEQ ID NOs. 1, 4, 45, 47, 49 and 51.

According to still further features in the described preferred embodiments the nucleotide sequence encodes, at most, about 70 amino acids of cadherin.

As used herein in the specification and in the claims section below, the term “about” refers to the range of ±20%.

According to still further features in the described preferred embodiments the nucleotide sequence encodes a β-catenin binding domain.

According to yet another aspect of the present invention there is provided a gene therapy vehicle harboring the above polynucleotide. Such a gene therapy vehicle is useful in the preparation of a pharmaceutical composition. Therefore, according to yet another aspect of the invention, there is provided a pharmaceutical composition comprising the above gene therapy vehicle of claim 7. Such a pharmaceutical composition is useful for treatment of cancer associated with abnormally high levels of β-catenin.

According to yet another aspect of the present invention there is provided a polypeptide comprising an amino acid sequence of a cytoplasmic portion of cadherin.

According to further features in preferred embodiments of the invention described below, the amino acid sequence includes a portion of a SEQ ID NO. selected from the group consisting of SEQ ID NOs. 2, 5, 46, 48, 50 and 52.

According to still further features in the described preferred embodiments the amino acid sequence includes, at most, about 70 amino acids of cadherin.

According to still further features in the described preferred embodiments the amino acid sequence includes a β-catenin binding domain.

According to yet another aspect of the present invention there is provided a pharmaceutical composition comprising the above polypeptide, which composition is useful for treatment of cancer associated with abnormally high levels of β-catenin.

According to still further features in the described preferred embodiments the cadherin is from a species selected from the group consisting of human, chicken, Xenopus, mouse, canine and Drosophila and other species known to express cadherin.

According to still further features in the described preferred embodiments the cadherin is selected from the group consisting of E-cadherin, N-cadherin, P-cadherin and VE-cadherin.

According to yet another aspect of the present invention there is provided a method of treating cancer associated with abnormally high levels of β-catenin comprising the step of treating the cancer with a therapeutic composition including a polypeptide, the polypeptide including a β-catenin binding domain, the polypeptide being therapeutically effective in reducing the abnormally high levels of β-catenin.

According to another aspect of the present invention there is further provided a pharmaceutical composition for treatment of cancer associated with abnormally high levels of β-catenin comprising a therapeutically effective amount of a polypeptide including a β-catenin binding domain, the polypeptide being therapeutically effective in reducing the abnormally high levels of β-catenin.

According to yet another aspect of the present invention there is further provided a method of treating cancer associated with abnormally high levels of β-catenin comprising the steps of genetically treating cancer cells with an acceptable gene therapy vehicle harboring a polynucleotide sequence encoding a polypeptide including a β-catenin binding domain, the polypeptide being therapeutically effective in reducing the abnormally high β-catenin transactivation activity.

According to still another aspect of the present invention there is further provided a pharmaceutical composition for treatment of cancer associated with abnormally high levels of β-catenin comprising an acceptable gene therapy vehicle harboring a polynucleotide sequence encoding a polypeptide including a β-catenin binding domain, the polypeptide being therapeutically effective in reducing the abnormally high levels of β-catenin transactivation activity.

According to further features in preferred embodiments of the invention described below, the polypeptide is a cytoplasmic portion of cadherin or a portion thereof.

According to still further features in the described preferred embodiments the cadherin is human.

According to still further features in the described preferred embodiments the cadherin is selected from the group consisting of E-cadherin, N-cadherin, P-cadherin and VE-cadherin.

According to still further features in the described preferred embodiments the cytoplasmic portion of cadherin or the portion thereof is signal peptide free.

According to still further features in the described preferred embodiments the polypeptide includes, at the most, about 70 amino acids of the β-catenin binding domain.

According to still further features in the described preferred embodiments the polypeptide includes, at the most, about 70 amino acids derived from the cadherin.

According to still further features in the described preferred embodiments the amino acids are derived from a carboxy terminus of the cadherin.

According to still further features in the described preferred embodiments the polypeptide is o-catenin or a portion thereof.

According to still further features in the described preferred embodiments the o-catenin is human.

The present invention successfully addresses the shortcomings of the presently known configurations by providing novel methods and therapeutic compositions for the combat in cancer.

### BRIEF DESCRIPTION OF THE DRAWINGS

The invention herein described, by way of example only, with reference to the accompanying drawings, wherein:

FIG. 1 is a schematic representation of cadherin constructs used in this study. Full length chicken N-cadherin and a chimera consisting of the extracellular and transmembrane domains of IL2Rα and the intracellular domain of N-cadherin (amino acids 752-912) (ILR/N-cad) are shown. The cytoplasmic domain of N-cadherin was tagged with a Flag epitope (N-cad (tail)), or fused to the C-terminus of GFP (NT). Two fragments of the cytoplasmic tail of N-cadherin (N71, amino acids 842-912 and N30, amino acids 862-891) were also fused to GFP. The cytoplasmic tail of mouse E-cadherin (amino acids 735-844) (ET) and two fragments from this domain (E72, amino acids 813-844 and E30, amino acids 833-862) were also fused to the C-terminus of GFP.

FIGS. 2A and 2B demonstrate stabilization of β-catenin by N-cadherin derivatives. (A), Western blot analysis of proteins from CHO cells (control), and CHO cells stably expressing full length N-cadherin (N-cad), the IL2R/N-cadherin chimera (IL2R/N-cad), or the N-cadherin cytoplasmic tail (N-cad (tail)) with antibodies against N-cadherin (lanes 1 and 2), the IL2R (lane 3) and Flag (lane 4). An identical blot was probed with anti β-catenin antibody. Note that β-catenin levels are higher in cells expressing the cadherin derivatives. (B) Co-immunoprecipitation (IP) analysis with anti cadherin antibody (cad) of extracts from CHO cells (lane 1) and from cells transfected with N-cadherin (lane 2), with anti IL2R antibody from cells transfected with the IL2R/N-cadherin chimera (IL2R, lane 3), or anti flag antibody with CHO cells transfected with the N-cadherin tail (flag, lane 4). Immunoblot analysis (IB) of the immunoprecipitates was performed using anti cadherin or anti β-catenin antibodies. The bands around 30 kDa and 50 kDa in all lanes are immunoglobulin chains from the IP. Note the co-precipitation of β-catenin with the various cadherin derivatives.

FIG. 3 demonstrate Triton X-100 solubility of proteins from CHO cells expressing N-cadherin and the N-cadherin cytoplasmic tail. Triton X-100 -soluble (sol) and -insoluble (ins) fractions of cell extracts were prepared and subjected to Western blot analysis with anti cadherin or anti flag antibodies. In CHO cells expressing N-cadherin (N-cad) 71% of the cadherin and 72% of β-catenin were found in the Triton-insoluble fraction. In contrast, in CHO cells transfected with the N-cadherin tail (N-cad (tail)) 74% of the cadherin and 64% of β-catenin were found in the detergent-soluble fraction.

FIG. 4 demonstrates localization of N-cadherin derivatives and β-catenin in CHO cells. Immunofluorescence staining of CHO cells and CHO expressing N-cadherin (N-cad), IL2Rα/N-cad, or the N-cadherin tail (N-cad tail). Cadherin was stained with secondary antibody conjugated to FITC, β-catenin with Cy3, and the nuclei with DAPI. Note that while CHO cells were only poorly stained for cadherin and β-catenin, CHO-N-cad cells displayed colocalization of N-cadherin and β-catenin in adherens junctions. CHO-IL2R/N-cad cells showed staining of N-cadherin and β-catenin in the membrane and cytoplasm, while CHO-N-cad tail cells exhibited nuclear staining for both the N-cadherin tail and β-catenin.

FIGS. 5A-5E demonstrate the effect of cadherin derivatives on β-catenin-mediated transactivation. CHO cells were transfected with TOPFLASH or FOPFLASH together with a cDNA encoding β-galactosidase (to normalize for transfection efficiency), β-catenin, LEF-1 and different cadherin derivatives in the indicated combinations (E), and transactivation was determined as the level of luciferase activity driven by a TOPFLASH containing construct (A). The levels of β-catenin (B), LEF-1 (C) and cadherin derivatives (D) in the different transfections were determined by Western blotting. Note the high level of β-catenin in the last three transfections (lanes 7-9) resulting from the stabilization of the endogenous β-catenin by the transfected cadherin derivatives. This pool of β-catenin however, was not available for transactivation.

FIGS. 6A-6B demonstrate inhibition of the constitutive LEF-1 responsive transactivation in SW480 cells by cadherin derivatives. (A) SW480 cells were transfected with TOPFLASH and the different cadherin constructs. The levels of β-catenin, LEF-1 and cadherins in the different transfections were determined by Western blot analysis. Note that the full length cadherin, the IL2R/N-cad chimera and the N-cad (tail) could all inhibit β-catenin-mediated transactivation (lanes 2-4 compare to lane 1). (B) Subcellular localization of N-cadherin N-CAD) (a), N-cadherin tail (N-CAD tail) and β-catenin (β-CAT) in SW480 cells transfected with the these cadherin-constructs. The bar represents 10 μm. Note that in cells transfected with full length N-cadherin, β-catenin re-localized from the nucleus to the cytoplasm and the plasma membrane, while the N-cadherin tail co-localized with β-catenin in the nucleus.

FIGS. 7A-7C demonstrate the effect of cadherin tail constructs on β-catenin level and transactivation. (A) CHO cells were transiently transfected with the various GFP-cadherin constructs (see FIGS. 1), and cell extracts were analyzed by Western blotting using anti GFP antibody. (B) The effect on β-catenin levels was determined by probing the same blot with anti β-catenin antibody. Note that the cadherin tail (NT) and the N71 and E72 cadherin tail fragments could all protect β-catenin from turnover, while the N30 and E30 cadherin fragments did not increase β-catenin levels. (C) SW480 cells were transfected with the indicated constructs and LEF-1-driven transactivation was determined as described in FIGS. 6A. Note that N71 and E72 could inhibit transactivation when compared to control (TOP), albeit less efficiently than the full length cytoplasmic tails of N- and E-cadherin.

FIGS. 8A-8E demonstrate competition between the N-cadherin tail and LEF-1 for binding to β-catenin. CHO cells were transfected at 1:1 ratio with cDNAs encoding β-catenin and HA-tagged LEF-1 (HA=hemaglutinin), together with increasing amounts of the N-cadherin tail. LEF-1 was immunoprecipitated (IP) using anti HA antibody and the levels of β-catenin (A) and LEF-1 (B) were determined by Western blotting (IB) using anti β-catenin and HA antibodies, respectively. Levels of N-cadherin tail (C) and β-catenin (D) in the transfected cells were determined by Western blot analysis (IB) of total protein extracts using anti cadherin and anti β-catenin antibodies. (E) Quantitative determination of the changes in the levels of the proteins shown in (A-D). Note that less β-catenin was co-precipitated in complex with LEF-1 when higher levels of cadherin tail were expressed, in spite of the presence of more β-catenin in the cells under these conditions (D).

FIG. 9 is a schematic representation of additional constructs used in this study. The molecules were tagged either with the hemaglutinin tag (HA) at the NH₂-terminus, or with the vesicular stomatitis virus - G (VSV-G) protein tag (VSV) at the COOH-terminus. Numbers 1-13 represent armadillo repeats in β-catenin, armadillo and plakoglobin with a non repeat region (ins) between repeats 10 and 11. Mutant plakoglobin and β-catenin lacking the COOH-transactivation domain (HA plakoglobin 1-ins; HA β-catenin 1-ins) were also constructed. A HA-tagged α-catenin that lacks the β-catenin binding domain was also prepared (HA (α-catenin Δβ). The COOH-terminal (C-term) transactivation domains of β-catenin and plakoglobin were fused to the DNA binding domain of Gal4 (Gal4DBD) (DBD=DNA-binding domain) to allow assessment of their transactivation potential.

FIGS. 10A-10F demonstrate nuclear localization of β-catenin and plakoglobin transiently transfected into MDCK cells. MDCK cells transfected with VSV-tagged β-catenin (β-CAT; A-C and F) or VSV-tagged plakoglobin (PG; D and E) were immunostained with either monoclonal anti β-catenin antibody (A), anti plakoglobin antibody (D), or anti VSV-tag antibody (B, C, E and F) and Cy3-labeled secondary antibody, 36 hours after transfection. The bar in (C) represents 10 μm. Note the nuclear localization of β-catenin and plakoglobin when overexpressed at high levels in MDCK cells (A-E), and of β-catenin at junctions when expressed at low level (F).

FIGS. 11A-11F demonstrate electronmicroscopical characterization of β-catenin and vinculin-containing nuclear structures in β-catenin-transfected cells. 293-T cells transfected with β-catenin were fixed and processed for (A), conventional TE microscopy, or the β-catenin-induced nuclear structures were identified by cryo EM with antibodies to β-catenin (B, C) or vinculin (D) using secondary antibodies bound to 10 nm gold particles. The nuclear structures were also visualized by phase (E) and immunofluorescence with anti β-catenin antibodies (F). The arrows in (E) point to nuclear structures decorated by anti β-catenin antibody (F). Nu, nucleus. The bars in (A, C and D) represent 0.2 μm, in (B), 1 μm, and in (F), 10 μm.

FIGS. 12A-12J demonstrate nuclear translocation of vinculin in β-catenin transfected cells. MDCK cells were transfected with VSV-tagged β-catenin and doubly stained with antibodies to the VSV tag (A, C, E and I) or to β-catenin (G), and with antibodies to LEF-1 (B), vinculin (D), α-catenin (F), plakoglobin (H), or α-actinin (J). Note the strong co-staining of LEF-1 and vinculin with β-catenin-containing nuclear rods, but not of plakoglobin, β-actinin or β-catenin. The bar in (I) represents 10 μm.

FIGS. 13A-13F demonstrate plakoglobin overexpression causes nuclear accumulation of β-catenin. Cells transfected with plakoglobin were doubly stained for plakoglobin (A, C and E), LEF-1 (B), β-catenin (D), or α-actinin (F). Note that in plakoglobin transfected cells β-catenin is translocated into the nucleus. α-ACT, α-actinin; β-cat, β-catenin; PG, plakoglobin. The bar in (E) represents 10 μm.

FIGS. 14A-14D demonstrate induction of nuclear translocation of β-catenin in stably transfected cells. Control neo^{r} HT1080 cells (A and B), and HT1080 cells stably transfected with an NH₂-terminal deleted β-catenin mutant (ΔN57; C and D) were either left untreated (A and C), or treated overnight with sodium butyrate (B and D) to enhance the expression of the transgene. Note the elevation in β-catenin content and its nuclear accumulation in butyrate-treated cells stably expressing ΔN57 β-catenin. The bar in (C) represents 10 μm.

FIGS. 15A-15H demonstrate nuclear translocation of β-catenin but not plakoglobin by LEF-1 overexpression. MDCK cells were transfected with either LEF-1 (A-D), with LEF-1 together with β-catenin (E and F), or with LEF-1 and plakoglobin (G-H). The cells were doubly stained with antibodies against LEF-1 (A, C, E and G) and antibodies to β-catenin (B) or plakoglobin (D). In doubly transfected cells (E-H), the transfected β-catenin (F) and plakoglobin (H) were detected by anti VSV-tag antibody. Note that LEF-1 efficiently translocated endogenous β-catenin into the nucleus, but not plakoglobin, while in cells transfected with both LEF-1 and plakoglobin or β-catenin, both transfected molecules were localized in the nucleus. The bar in (G) represents 10 μm.

FIGS. 16A and 16B demonstrate differential Triton X-100 solubility of various junctional plaque proteins and nuclear translocation of vinculin in cells overexpressing β-catenin together with LEF-1. (A) Equal volumes of total MDCK cell proteins (T), and Triton X-100-soluble (S) and -insoluble (I) cell fractions, were analyzed by gel electrophoresis and Western blotting with antibodies to β-catenin (β-CAT), plakoglobin (PG), vinculin (vinc), α-actinin (a-Act) and α-catenin (α-cat). Note that while β-catenin, vinculin and α-catenin present a large pool of a detergent-soluble fraction, plakoglobin and α-actinin are almost entirely insoluble in Triton X-100. (B) MDCK cells were co-transfected with LEF-1 and β-catenin and doubly stained for β-catenin (β-CAT, upper inset) and vinculin (Vinc), and β-catenin (β-CAT, lower inset) and plakoglobin (PG). Note that in cells doubly transfected with β-catenin and LEF-1 vinculin translocated into the nucleus, but plakoglobin remained junctional. The bar represents 10 μm.

FIGS. 17A-17D demonstrate elevation of β-catenin and plakoglobin content and nuclear localization after treatment with inhibitors of the ubiquitin-proteasome pathway. (A) Balb/C 3T3 cells were untreated (c) or treated for 4 hours with inhibitors of the ubiquitin-proteasome system: Lactacystin (Lact), ALLN (N-Acetyl-Leu-Leu-Norleucinal), or MG-132, and equal amounts of protein were analyzed by Western blotting with anti β-catenin antibody. (B), KTCTL60 and KTCTL60-PG cells (stably overexpressing plakoglobin) were treated with 10 and 20 μM MG-132 and probed with anti β-catenin and plakoglobin antibodies. (C) Northern blot hybridization for β-catenin and plakoglobin in KTCTL60, KTCTL60-PG and MDCK cells. (D), Balb/C 3T3 and KTCTL60 cells (a, c and e), were treated for 4 hours with MG-132 (b, d and f) and stained with antibodies to β-catenin (a-d) or plakoglobin (e and f). Note the appearance of higher molecular weight β-catenin forms in 3T3 cells (bracket in A), the dramatic elevation in β-catenin content of KTCTL60 cells, and the moderate increase in plakoglobin after treatment of KTCTL60-PG with the proteasome inhibitors.

FIGS. 18A-18B demonstrate activation of Gal4- and LEF-1-driven transcription by β-catenin and plakoglobin. (A), Constructs consisting of the DNA-binding domain of Gal4 (Gal4DBD) fused to the COOH-terminal-transactivation domains of β-catenin and plakoglobin were co-transfected with a reporter gene (luciferase) driven by Gal4-responsive sequences into 3T3 cells, and the levels of luciferase activity determined from duplicate transfections (light and dark bars). (B), Transactivation of LEF-1 consensus sequence (TOPFLASH)-driven transcription by full length and truncated β-catenin and plakoglobin in 293 cells. The values (fold increase) were normalized for transfection efficiency by analyzing β-galactosidase activity of co-transfected lacZ, and for LEF-1 specificity with an inactive mutant LEF-1 sequence (FOPFLASH, light bars). (C), Double immunofluorescence for β-catenin (insets a, d and f) in cells transfected with plakoglobin (inset a), β-catenin 1-ins (inset c), and plakoglobin 1-ins (inset e). Note that chimeras consisting of β-catenin and plakoglobin fused to Gal4 DNA-binding domain were both active in transcription stimulation, but LEF-1-responsive transactivation by β-catenin (and a β-catenin mutant) was much more potent than by full length plakoglobin, and a COOH-deletion mutant of plakoglobin was inactive in LEF-1-driven transactivation. Full length plakoglobin and the β-catenin mutant (β-cat 1-ins) were effective in translocating endogenous β-catenin into the nucleus, while the plakoglobin mutant (PG 1-ins) was not.

FIGS. 19A and 19B demonstrate inhibition of transactivation and nuclear accumulation of β-catenin in SW480 colon carcinoma cells after transfection with N-cadherin or α-catenin. (A) SW480 cells were transfected with either empty vector (pCGN), N-cadherin, α-catenin, or a mutant α-catenin lacking the β-catenin binding site (HA α-catenin Δβ, FIG. 9) together with a multimeric LEF-1 binding consensus sequence driving the expression of luciferase. The values of luciferase expression were corrected for transactivation specificity with a mutant LEF-1 consensus sequence, and with β-galactosidase activity for transfection efficiency. (B) Cells were transfected with N-cadherin (insets a and b), α-catenin (insets c and d), or mutant α-catenin (α-CATΔβ) (insets e and f) and doubly stained for β-catenin (insets b, d and f) and N-cadherin (inset a), α-catenin (inset c) and mutant α-catenin (inset e). Note the inhibition of transactivation and cytoplasmic retention of β-catenin in cells transfected with N-cadherin- or α-catenin, but not with mutant α-catenin. The bar represents 10 μm.

FIGS. 20-23 demonstrate sequence homologies among the cytoplasmic tail encoding portion of representative types of cadherin (CAD) genes, cytoplasmic tails of representative types of cadherin proteins, representative types of o-catenin genes and representative types of o-catenin proteins, respectively.

### DESCRIPTION OF THE PREFERRED EMBODIMENTS

The present invention is of methods and therapeutic compositions which can be used for the treatment of cancer. Specifically, the present invention is of methods employing, and of therapeutic compositions including, peptides featuring β-catenin binding domains, or polynucleotide sequences encoding same, for use in the treatment of cancers associated with abnormally high levels of β-catenin transactivation activity, such as, but not limited to, colon cancers (carcinomas) and melanomas.

The principles and operation of the methods and compositions according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

We studied the effect of N-cadherin, its cytoplasmic domain and a transmembrane chimeric molecule containing the cadherin cytoplasmic tail, on the level and localization of P-catenin and its ability to induce LEF-1-responsive transactivation. These cadherin derivatives formed complexes with β-catenin protecting it from degradation. N-cadherin directed β-catenin into adherens junctions, the chimeric protein and the associated β-catenin were diffusely distributed on the membrane, while the cytoplasmic domain of N-cadherin colocalized with β-catenin in the nucleus. Co-transfection of β-catenin and LEF-1 induced transactivation of a LEF-1 reporter. In CHO cells the N-cadherin-derived molecules blocked β-catenin-driven transactivation. Expression of N-cadherin and the IL-2 receptor/cadherin chimera in SW480 cells relocated β-catenin from the nucleus to the plasma membrane, and reduced transactivation. The cytoplasmic tail of N- or E-cadherin co-localized with β-catenin in the nucleus, and suppressed the constitutive LEF-1-mediated transactivation, by blocking β-catenin-LEF-1 interaction. Moreover, the 72 C-terminal amino acids of cadherin stabilized β-catenin and inhibited its transactivation potential. These results indicate that β-catenin binding to the cadherin cytoplasmic tail either in the membrane, or in the nucleus, can inhibit β-catenin degradation and efficiently block its transactivation capacity.

As already mentioned, β-Catenin and plakoglobin are homologous proteins that function in cell adhesion by linking cadherins to the cytoskeleton and in signaling by transactivation together with LEF/TCF transcription factors. Here the nuclear translocation and transactivation abilities of β-catenin and plakoglobin in mammalian cells are compared. Overexpression of each of the two proteins in MDCK cells resulted in nuclear translocation and formation of nuclear aggregates. The β-catenin-containing nuclear structures also contained LEF-1 and vinculin, while plakoglobin was inefficient in recruiting these molecules suggesting that its interaction with LEF-1 and vinculin is significantly weaker. Moreover, transfection of LEF-1 translocated endogenous β-catenin, but not plakoglobin to the nucleus. Chimeras consisting of Gal4 DNA-binding domain and the transactivation domains of either plakoglobin or β-catenin were equally potent in transactivating a Gal4-responsive reporter, while activation of LEF-1-responsive transcription was significantly higher with β-catenin. Overexpression of wt plakoglobin or mutant β-catenin lacking the transactivation domain induced accumulation of the endogenous β-catenin in the nucleus and LEF-1-responsive transactivation. It is further shown that the constitutive β-catenin-dependent transactivation in SW480 colon carcinoma cells and its nuclear localization can be inhibited by overexpressing N-cadherin or α-catenin. The results indicate that (i) plakoglobin and β-catenin differ in their nuclear translocation and complexing with LEF-1 and vinculin; (ii) LEF-1-dependent transactivation is preferentially driven by β-catenin; (iii) the cytoplasmic partners of β-catenin, cadherin and α-catenin, can sequester it to the cytoplasm and inhibit its transcriptional activity.

β-Catenin interacts with three major subcellular systems that affect its activities and fate, including: (a) adherens-type junctions, where β-catenin forms a complex with the cytoplasmic domain of different cadherins and links them to the actin cytoskeleton; (b) a unique degradation system that regulates the level of β-catenin via a multi-step process that includes binding to APC, phosphorylation by GSK-3β and degradation by the ubiquitin-proteasome system; and (c) the transcriptional machinery, where β-catenin interacts with LEF/TCF transcription factors and activates the expression of specific target genes.

While reducing the present invention into practice the cross-talk between these systems were investigated, and in particular, the effect of cadherin and cadherin derivatives on β-catenin stability and signaling capacity characterized.

Elevated expression of cadherin could potentially protect β-catenin from degradation, increasing its level in the cytoplasm and therefore stimulating LEF-1-responsive transcription. However, the same “protective cadherin” could also block β-catenin-mediated transactivation either by sequestering β-catenin to the plasma membrane (away from the nucleus), and/or competing with transcription factors of the LEF/TCF family that interact with β-catenin. It is shown that cadherin can indeed translocate β-catenin to junctional sites and stabilize the protein against degradation in cells where a “normal” (rapid) turnover of β-catenin takes place. This recruitment of β-catenin to junctional sites is accompanied by a strong inhibition of LEF-1-directed transcription. The presence of organized junctions however, was not essential for neither membrane translocation of β-catenin, nor for stabilization and inhibition of β-catenin-mediated transactivation. A chimeric receptor consisting of the cadherin cytoplasmic domain and an inert transmembrane anchor (IL2R) was fully effective regarding these functions, despite being unable to participate in junction formation, and its capacity to inhibit junction assembly in cadherin-containing cells. Interestingly, the cytoplasmic tails of both E- and N-cadherin were most effective in protecting β-catenin from degradation and inhibiting its transactivating potential, but did not affect the subcellular distribution of β-catenin. Thus, in cells expressing high levels of both β-catenin and the cadherin tail, the two proteins co-localized in the nucleus, and β-catenin-driven transcription was strongly suppressed. The most likely explanation for this effect is that binding of the cadherin tail (either isolated, or as part of the intact cadherin molecule) to β-catenin, inhibited the binding of β-catenin to LEF-1 and subsequently transactivation. This notion is supported by the co-immunoprecipitation experiments demonstrating that increasing levels of the cadherin tail resulted in lower levels of β-catenin-LEF-1 complex formation. The mechanism whereby N-cadherin or its tail confer the stabilization of β-catenin could also result from an effective competition with β-catenin binding to APC, or to other components of the APC-GSK-3β-ubiquitin-proteasome systems.

The current information on the binding sites for cadherin, APC and LEF-1 on β-catenin indicates that multiple overlapping armadillo repeats are involved: Armadillo repeats 4-13 are important for E-cadherin binding, repeats 1-10 are involved in APC binding, repeats 3-8 are essential for β-catenin-dTCF interaction, while repeats 1-14 are involved in β-catenin-LEF-1 interaction. Taken together with the present study, the association of β-catenin with each of these components appears to be mutually exclusive.

It is also demonstrated herein that sequestration to the plasma membrane or to adherens junctions is not necessary for protecting β-catenin from degradation, since the soluble N-cadherin tail did not affect the nuclear localization of β-catenin while being efficient in stabilizing β-catenin in CHO cells (similar to full length cadherin). As it was found that the soluble cadherin tail was capable of efficiently competing with LEF-1 for β-catenin binding in the nucleus, β-catenin translocation into the nucleus may not require LEF-1, in agreement with a recent report showing a role for the importin/karyophilin system in this process. Since shorter fragments of the cadherin tail could inhibit the constitutive transcriptional activity of β-catenin in SW480 cells, the antagonistic effect of cadherin on β-catenin signaling in these human cancer cells is most probably independent of adherens junction formation, similarly to the results obtained for β-catenin-signaling in Drosophila and Xenopus. However, since this inhibition of transcription only occurred after artificially increasing cadherin levels in these cells, its physiological significance remains unclear.

The results presented here are also relevant to some novel approaches aiming to suppress β-catenin-driven oncogenesis. It was demonstrated that mutations in APC that cannot participate in β-catenin degradation result in high levels of β-catenin in colon carcinoma, and in the activation of genes (the nature of which is still unknown) that are probably involved in the transformation of these cells. Thus, the cytoplasmic cadherin tail and fragments derived from it (that contain the β-catenin-binding site), may prove to be useful in blocking the expression of such target genes and therefore suppressing tumorigenicity. This study indicates that the C-terminal region of E and N-cadherin, corresponding to approximately 70 amino acids retains the transactivation suppressive capacity.

As mentioned, in mammalian cells, β-catenin and the closely related molecule plakoglobin have been shown to complex independently with similar partners, and are both involved in the formation of adherens type junctions. Plakoglobin, in addition, can associate with various desmosomal components, while β-catenin does not normally associate with desmosomes, except in plakoglobin-null mouse embryos where the segregation between adherens junctions and desmosomes collapses. Plakoglobin is also unable to substitute for β-catenin during development, as β-catenin-null mouse embryos die early in development. In this study some common features of β-catenin and plakoglobin are highlighted, as well as considerable differences in their nuclear translocation under various conditions, and their capacity to function in transcriptional activation. For both proteins, the increase in free protein levels induces nuclear translocation. This translocation can be blocked by junctional proteins which bind to β-catenin and sequester it to the plasma membrane or the cytoplasm.

Under the various conditions that resulted in increased levels of β-catenin and plakoglobin, both proteins translocated into the nucleus independently of, or in complex with LEF-1. While in β-catenin overexpressing cells the nuclear complexes that were formed by excess β-catenin also contained vinculin, in addition to LEF- 1, plakoglobin overexpression did not result in the recruitment of these molecules into the nuclear speckles. Interestingly, α-catenin and α-actinin both of which bind β-catenin and plakoglobin-containing complexes, were not co-translocated into the nucleus by β-catenin or plakoglobin probably due to their stronger binding to actin filaments resulting in a limited soluble pool in cells, in contrast to vinculin that is mostly in the detergent-soluble fraction. The present study is the first demonstration that ,-catenin can associate with and recruit into the nucleus vinculin, but not other components of the cadherin-catenin system (i.e. α-catenin, α-actinin, or cadherin) in a complex that also contains LEF-1. The association between vinculin and β-catenin was recently demonstrated by co-immunoprecipitation of these proteins together with E-cadherin, but was most pronounced in cells lacking α-catenin. Taken together, these findings reveal a new interaction of β-catenin with vinculin, that under certain conditions may lead to their colocalization in the nucleus where such complex may play an important physiological role, yet to be determined.

While both plakoglobin and β-catenin exhibited a largely similar nuclear translocation, they were distinct in their ability to colocalize with LEF-1 in the nucleus. Endogenous β-catenin was readily translocated into the nucleus following transfection with LEF-1, in agreement with previous studies, whereas the endogenous plakoglobin remained junctional. This difference may be attributed to the availability of a larger pool of soluble β-catenin in MDCK cells, or to an intrinsic difference between the two molecules in their binding to LEF-1.

Plakoglobin and β-catenin also differed in their ability to influence the localization of endogenous LEF-1 when individually overexpressed. Plakoglobin overexpression could drive part of the endogenous β-catenin into the nucleus, most probably by displacing it from cadherin or other cytoplasmic partners, in agreement with results obtained with HT1080 cells and with Xenopus embryos. This implies that plakoglobin may have a regulatory role in the control of the extrajunctional function of β-catenin. In contrast, β-catenin was inefficient in altering plakoglobin's localization in MDCK, 293 and SK-BR-3 cells (all expressing desmosomes, unpublished results). This was partly expected since β-catenin is not normally associated with desmosomes and the soluble pool of plakoglobin in these cells is very low.

Plakoglobin and β-catenin also responded differently to the inhibition of the ubiquitin-proteasome pathway, in particular in the renal carcinoma cell line KTCTL60 that does not express detectable levels of proteins of the cadherin-catenin system. The level of β-catenin could be dramatically induced in these cells with proteasome inhibitors, suggesting that efficient degradation of β-catenin is responsible for the very low level of β-catenin in these cells. Plakoglobin was absent from these cells, as there was no plakoglobin RNA, but when it was stably expressed, its level was only moderately enhanced by inhibitors of the ubiquitin-proteasome system. It is interesting to note that this stable expression of plakoglobin did not result in the elevation of β-catenin content in KTCTL60 cells, implying that plakoglobin cannot, by itself, effectively protect β-catenin from degradation in cells lacking cadherins. Only when the level of plakoglobin was further increased in these cells by butyrate treatment, could some accumulation of β-catenin be detected.

Comparison between the presumptive transactivating domains of β-catenin and plakoglobin, fused to the DNA-binding domain of Gal4, indicated comparable transcriptional activation by the two molecules. This demonstrated that plakoglobin like β-catenin and armadillo, has a potent transactivation domain. However, the specific transcriptional activation of LEF-1-driven reporter gene by plakoglobin was several fold less efficient than that of β-catenin. Interestingly, a deletion mutant of β-catenin that lacked the transactivating domain but retained the cadherin binding domain, was also capable of inducing transcription by the LEF-1 consensus construct. This can be attributed to competition and displacement of endogenous β-catenin from a complex with its cytoplasmic partners, nuclear translocation of the endogenous β-catenin and consequently LEF-1-driven transactivation. This finding is in agreement with studies demonstrating that a variety of membrane-anchored mutant forms of β-catenin can act in signaling for axis duplication in Xenopus embryos by releasing endogenous β-catenin from cell-cell junctions or from a complex with APC thus enabling its translocation into the nucleus.

Overexpression of full length plakoglobin was capable of inducing nuclear translocation of the endogenous β-catenin in MDCK and 293 cells, while a COOH-terminus mutant plakoglobin, previously shown to be inefficient in displacing β-catenin from its complex with cadherin, was unable to induce nuclear localization of the endogenous β-catenin, or transcriptional activation of the LEF-1-driven reporter. Since plakoglobin overexpression was inefficient in driving LEF-1 to complex with the nuclear speckles formed by plakoglobin overexpression, it is conceivable that the majority of the transcriptional stimulation of LEF-1-driven transcription in plakoglobin overexpressing cells was due to the endogenous β-catenin that relocated to the nucleus under these conditions.

Another recent study examining mammalian β-catenin and plakoglobin's embryonic signaling abilities in Drosophila (rescue of the segment polarity phenotype of armadillo) suggested that while both proteins can rescue armadillo mutants in adhesion properties, β-catenin had only a weak- and plakoglobin had no detectable -signaling activity. Nevertheless, since is was found that the COOH-terminus of plakoglobin is potent in transcriptional activation in the Gal4-fusion chimera and deletion mutants at the COOH terminus were inefficient in transactivation and most of the overexpressed plakoglobin was localized in the nuclei of transfected cells, one cannot exclude, at this point, the possibility that plakoglobin can also play a direct role in the transcriptional regulation of specific genes that are yet to be identified. This possibility is being currently examined by analyzing the transactivation capacities of plakoglobin and β-catenin in cells that lack such endogenous proteins.

In the human colon carcinoma SW480 cells which lack APC and therefore accumulate abnormally high levels of β-catenin in the nuclei, transcriptional activation of the LEF-1-driven reporter could be inhibited by members of the cadherin-catenin complex that sequestered β-catenin to the cytoplasm.

The role of cadherin in regulating β-catenin levels is complex: On the one hand, elevation in the content of cadherin can protect β-catenin from degradation and increase its levels. On the other hand, a strong binding of β-catenin to cadherin, rather than to LEF- 1, may result in its cytoplasmic sequestration and the inhibition of transactivation by it. Furthermore, the cytoplasmic tail of cadherin that contains the binding site for β-catenin can also inhibit transactivation by β-catenin even when bound to it in the nucleus of the transfected cells. The association of β-catenin with overexpressed α-catenin in SW480 cells also resulted in the cytoplasmic retention of nuclear β-catenin by binding of α-catenin to the actin-cytoskeleton. These results are in agreement with those obtained for β-catenin signaling in axis specification of developing Xenopus that is antagonized by overexpression of cadherin, or by the NH₂-terminus of α-catenin.

These results may have important implications for the possible role of β-catenin in the regulation of tumorigenesis, since E-cadherin and α-catenin were suggested to have tumor suppressive effects when re-expressed in cells deficient in these proteins, and were shown to affect the organization of cell-cell adhesion. In addition, modulation of vinculin and α-actinin levels in certain tumor cells was shown to influence the tumorigenic ability of these cells and to affect anchorage independence and tumorigenicity in 3T3 cells. It is possible that such effects are attributable to the capacity of vinculin and α-actinin to bind β-catenin thus affecting both its localization and its role in regulating transcription.

The β-catenin binding domains of cadherin and α-catenin are well characterized. It will therefore be possible to provide shorter peptides or peptidomimetics comprising these domains which are therapeutically effective in blocking the oncogenic action conferred by constitutive transactivation of LEF/TCF-responsive genes by β-catenin in colon cancer or other cancers.

In addition, gene therapy with suitable vectors including nucleic acid sequences encoding these therapeutically effective peptides may be used for treatment of cancer associated with the β-catenin transactivation system.

It will be appreciated that one ordinarily skilled in the art of proteinaceous drug design and delivery, would know how the design peptides or peptidomimetics therapeutically effective in treating β-catenin transactivation system associated cancers.

It will further be appreciated that one ordinarily skilled in the art of gene therapy, would know how the design vectors encoding these therapeutically effective peptides and to use such vectors in treating β-catenin transactivation system associated cancers.

U.S. Pat. No. 5,683,866, to Sarkar et al., entitled “process for producing a targeted gene”, and which is incorporated by reference as if fully set forth herein, discloses a reconstituted sendai-viral envelope containing the F-protein (F-virosomes) and to a process for producing a targeted gene or drug delivery carrier produced by the steps of chemical reduction of Sendai virus for reduction of HN protein and subjecting the reduced virus to the step of dialysis for removal of the reducing agent. The reduced virus is then solubilized with a detergent to obtain a solution. The said solution is centrifuged to separate the insolubles consisting of reduced HN protein and core of the virus, adding the required specific gene or drug to the centrifugal solution. Finally, the detergent is removed using an affinity complex agent which binds the detergent leading to the formation of the delivery carrier.

U.S. Pat. No. 5,455,027 to Zalipsky et al., entitled “poly(alkylene oxide) amino acid copolymers and drug carriers and charged copolymers based thereon”, and which is incorporated by reference as if fully set forth herein, teaches copolymers of poly(alkylene oxides) and amino acids or polypeptide sequences which have pendant functional groups that are capable of being conjugated with pharmaceutically active compounds for drug delivery systems and cross-linked to form polymer matrices functional as hydrogel membranes. The copolymers can also be formed into conductive materials. Methods are also disclosed for preparing the polymers and forming the drug conjugates, hydrogel membranes and conductive materials.

U.S. Pat. No. 5,652,130 to Kriegler et al., entitled “retroviral vectors expressing tumor necrosis factor (TNF)”, and which is incorporated by reference as if fully set forth herein, discloses a drug delivery virion which contains an expression system for the desired protein active ingredient packaged in an envelope derived from a retrovirus is especially useful in administering materials which need to cross cell membranes in order to serve their function.

U.S. Pat. No. 5,635,399 to Kriegler et al., entitled “retroviral vectors expressing cytokines”, and which is incorporated by reference as if fully set forth herein, similarly teaches a drug delivery virion which contains an expression system for the desired protein active ingredient packaged in an envelope derived from a retrovirus is especially useful in administering materials which need to cross cell membranes in order to serve their function.

U.S. Pat. No. 5,580,575 to Unger et al., entitled “therapeutic drug delivery systems”, and which is incorporated by reference as if fully set forth herein, teaches therapeutic drug delivery systems comprising gas-filled microspheres comprising a therapeutic are described. Methods for employing such microspheres in therapeutic drug delivery applications are also provided. Drug delivery systems comprising gas-filled liposomes having encapsulated therein a drug are preferred. Methods of and apparatus for preparing such liposomes and methods for employing such liposomes in drug delivery applications are also disclosed.

Different designs for gene therapy are also disclosed in Huber E., B. and Magrath I. 1998. Gene therapy in the treatment of cancer. Cambridge University Press., which is incorporated herein by reference.

Thus, in accordance with one aspect of the present invention there is provided a polynucleotide which comprises a nucleotide sequence encoding a cytoplasmic portion of cadherin. Preferably, the nucleotide sequence includes a portion of SEQ ID NOs. 1, 4, 45, 47, 49 or 51 and it preferably encodes, at most, about 70 amino acids of cadherin, preferably that portion of cadherin which includes a β-catenin binding domain.

Accordingly, there is also provided a gene therapy vehicle harboring the above described polynucleotide. Such a gene therapy vehicle is useful in the preparation of a pharmaceutical composition, itself, as further detailed hereinunder, is useful for treatment of cancer associated with abnormally high levels of β-catenin.

In accordance with another aspect of the present invention there is provided a polypeptide which comprises an amino acid sequence of a cytoplasmic portion of cadherin. Preferably, the amino acid sequence includes a portion of SEQ ID NOs. 2, 5, 46, 48, 50 or 52. Preferably, the amino acid sequence includes, at most, about 70 amino acids of cadherin, most preferably it includes the β-catenin binding domain of cadherin.

Accordingly there is provided a pharmaceutical composition comprising the above polypeptide, which composition is useful for the treatment of cancer associated with abnormally high levels of β-catenin.

The cadherin according to the present invention may be of any type and from any species. Types of cadherins include E-cadherin, N-cadherin, P-cadherin and VE-cadherin. Species include human, chicken, Xenopus, mouse, canine and Drosophila and other species known to express cadherin.

Further according to the present invention there is provided a method of treating cancer associated with abnormally high levels of β-catenin. According to the method the cancer is treated with a therapeutic composition containing a polypeptide which includes a β-catenin binding domain, wherein the polypeptide is therapeutically effective in reducing the abnormally high levels of β-catenin.

Accordingly, there is further provided a pharmaceutical composition for treatment of cancer associated with abnormally high levels of β-catenin. The pharmaceutical composition includes a therapeutically effective amount of a polypeptide including a β-catenin binding domain, therapeutically effective in reducing the abnormally high levels of β-catenin.

In accordance with yet another aspect of the present invention there is provided yet another method of treating cancer associated with abnormally high levels of β-catenin. According to this method the cancer cells are genetically treated with an acceptable gene therapy vehicle harboring a polynucleotide sequence encoding a polypeptide including a β-catenin binding domain, therapeutically effective in reducing the abnormally high levels of β-catenin.

Accordingly, there is further provided a pharmaceutical composition for treatment of cancer associated with abnormally high levels of β-catenin. This pharmaceutical composition includes an acceptable gene therapy vehicle harboring a polynucleotide sequence encoding a polypeptide including a β-catenin binding domain, therapeutically effective in reducing the abnormally high levels of β-catenin.

As used herein in the specification and in the claims section below, the term “treating” includes substantially inhibiting, slowing or reversing the progression of a disease, substantially ameliorating clinical symptoms of a disease or substantially preventing the appearance of clinical symptoms of a disease.

As used herein in the specification and in the claims section below, the term “β-catenin binding domain” refers to an amino acid sequence capable in specifically binding β-catenin. Few examples are given hereinbelow in the Examples section. However, the scope of the present invention is not limited to the specified examples. Processes for isolating binding domains are well known in the art. Examples include, but are not limited to, affinity column chromatography, use of an antibody specific to a protein in a protein complex to precipitate the protein complex, phage display library screening, etc. Using a genetic approach, the yeast two hybrid system can be employed to clone nucleic acid sequences encoding polypeptides having such domains (Ausubel S. M., et al. Eds. (1998, electronic version update) Current protocols in molecular biology. Willy & Sons. N.Y. electronic update).

As used herein in the specification and in the claims section below, the term “acceptable gene therapy vehicle” refers to any vector or composition of matter useful in in vivo introduction of nucleic acids into cells of an organism. Some examples are mentioned hereinabove.

According to a preferred embodiment of the invention the polypeptide is a cytoplasmic portion of cadherin or a portion thereof. As shown in the Examples section below, the cytoplasmic portion of cadherin includes a β-catenin binding domain capable of binding β-catenin and sequestering it from the nucleus and/or limit its association with LEF-1, and therefore effective in reducing the abnormally high levels of β-catenin. According to a preferred embodiment of the invention the cytoplasmic portion of cadherin or the portion thereof is signal peptide free.

As used herein in the specification and in the claims section below, the term “signal peptide” refers to an amino acid sequence known to, or capable of directing a protein to a membrane.

The cadherin is preferably human cadherin. However, as shown in the Examples section below, all cadherins share high amino acid sequence homology and are all known to bind β-catenin. Furthermore, interspecies (heterologous) experiments reported herein and in numerous publications demonstrate functional compatibility of cadherin and β-catenin across the animal kingdom, from Drosophila, through Xenopus to a variety of mammals and human. Therefore, the scope of the present invention is not limited to any specific type of cadherin, as it is well known that all cadherins effectively bind β-catenin. Known cadherins include, but are not limited to, E-cadherin, N-cadherin, P-cadherin and VE-cadherin from human, chicken, Xenopus, mouse, canine and Drosophila and other species known to express cadherins.

The β-catenin binding domain of each of these cadherins may be used in the therapeutic composition, and/or to effect the method, of the present invention.

According to a preferred embodiment of the present invention, the polypeptide includes, at the most, about 70 amino acids of the β-catenin binding domain. The polypeptide may include additional sequences or motives required, for example, for drug targeting or delivery. As used herein in the specification and in the claims section below, the term “about” refers to ±10%. According to a preferred embodiment of the present invention the polypeptide includes, at the most, about 70 amino acids derived from cadherin, preferably from the carboxy terminus thereof. As demonstrated in the Examples section below, the carboxy terminus of cadherin is highly effective in binding β-catenin.

Thus, according to a preferred embodiment of the invention the polynucleotide sequence encodes a cytoplasmic portion of cadherin or a portion thereof capable of binding β-catenin and sequestering it from the nucleus and/or limit its association with LEF-1, and therefore is effective in reducing the abnormally high levels of β-catenin. The scope of the present invention is not limited to any specific type of cadherin encoding polynucleotide, as it is well known that all cadherins effectively bind β-catenin. Known cadherin genes include, but are not limited to, E-cadherin, N-cadherin, P-cadherin and VE-cadherin from human, chicken, Xenopus, mouse, canine and Drosophila and other species known to express cadherins. The β-catenin binding domain encoded by each of these cadherin genes may be used in the therapeutic composition, and/or to effect the method, of the present invention.

According to a preferred embodiment of the present invention, the polynucleotide sequence encodes about 70 amino acids of the β-catenin binding domain, at most. However, it may encode additional sequences or motives required, for example, for stability. According to a preferred embodiment of the present invention the polynucleotide sequence encodes, at the most, about 70 amino acids derived from cadherin, preferably from the carboxy terminus thereof. As demonstrated in the Examples section below, the carboxy terminus of cadherin is highly effective in binding β-catenin.

According to another preferred embodiment of the present invention, the polypeptide is o-catenin or a portion thereof. Thus, according to another preferred embodiment of the present invention, the polynucleotide sequence encodes o-catenin or a portion thereof.

As shown in the Examples section below, the o-catenin, which is known to include a β-catenin binding domain, is capable of binding β-catenin and sequestering it from the nucleus and/or limit its association with LEF-1. Therefore o-catenin, or its β-catenin binding domain, are effective in reducing the abnormally high levels of β-catenin.

The o-catenin is preferably human o-catenin. However, as shown in the Examples section below, all o-catenins share high amino acid sequence homology and are all known to bind β-catenin. Furthermore, interspecies experiments reported herein and in numerous publications demonstrate functional compatibility of o-catenin and β-catenin across the animal kingdom, from Drosophila, through Xenopus to a variety of mammals and human. Therefore, the scope of the present invention is not limited to any specific type of o-catenin, as it is well known that all o-catenins effectively bind β-catenin. Known o-catenins include, but are not limited to, those of human, chicken, Xenopus, mouse, canine and Drosophila and other species known to express cadherins. The β-catenin binding domain of each of these o-catenins may be used in the therapeutic composition, and/or to effect the method, of the present invention.

The polynucleotide according to the present invention may be a native polynucleotide or alternatively it may be a therapeutically active mutant, variant or portion thereof. Furthermore, the polynucleotide may further include a fused sequence which encodes a polypeptide which may render the fused protein more stable under the harsh cellular environment. For example, the fused sequence can encode, for example, a signal peptide which will direct the fused (chimeric) protein to the plasmatic cell membrane.

The polypeptide according to the present invention may include the 20 naturally occurring amino acids; those amino acids often modified post-translationally in vivo, including for example hydroxyproline, phosphoserine and phosphothreonine; and other unusual amino acids including, but not limited to, 2-aminoadipic acid, hydroxylysine, isodesmosine, nor-valine, nor-leucine and ornithine, all in D or L configurations. Similarly, the polypeptide may be a peptidomimetic molecule, for example, prepared by peptidomimetic methods, which has similar binding properties to β-catenin. Methods for preparing peptidomimetic compounds are well known in the art and are specified in Quantitative Drug Design, C. A. Ramsden Gd., Chapter 17.2, F. Choplin Pergamon Press (1992). Specifically, peptidomimetic compounds can be prepared by replacing at least one peptide bond with various non-peptidic bonds, such as, but not limited to, CH₂—NH, CH₂—S, CH₂—S═O, O═C—NH, CH₂—O, CH₂—CH₂, S═C—NH, CH═CH or CF═CH. The polypeptide according to the present invention may be a native polypeptide or alternatively it may be a therapeutically active mutant, variant or portion thereof. Furthermore, the polypeptide may further include a fusion polypeptide which may serve to assist the therapeutically functional polypeptide to cross cell membranes.

For therapeutic treatment of cancer the polypeptide can be formulated in a pharmaceutical composition, which may include thickeners, carriers, buffers, diluents, surface active agents, preservatives, and the like, all as well known in the art. Pharmaceutical compositions may also include one or more active ingredients such as but not limited to antiinflammatory agents, antimicrobial agents, anesthetics and the like.

The pharmaceutical composition may be administered in either one or more of ways depending on whether local or systemic treatment is of choice, and on the area to be treated. Administration may be done topically (including ophtalmically, vaginally, rectally, intranasally), orally, by inhalation, or parenterally, for example by intravenous drip or intraperitoneal, subcutaneous, intramuscular or intravenous injection.

Formulations for topical administration may include, but are not limited to, lotions, ointments, gels, creams, suppositories, drops, liquids, sprays and powders. Conventional pharmaceutical carriers, aqueous, powder or oily bases, thickeners and the like may be necessary or desirable.

Compositions for oral administration include powders or granules, suspensions or solutions in water or non-aqueous media, sachets, capsules or tablets. Thickeners, diluents, flavorings, dispersing aids, emulsifiers or binders may be desirable. Slow release particles are also conceivable.

Formulations for parenteral administration may include but are not limited to solutions which may also contain buffers, diluents and other suitable additives.

Dosing is dependent on severity and responsiveness of the condition to be treated, but will normally be one or more doses per day, with course of treatment lasting from several days to several months or until a cure is effected or a diminution of disease state is achieved. Persons ordinarily skilled in the art can easily determine optimum dosages, dosing methodologies and repetition rates.

Reference is now made to the following examples, which together with the above descriptions, illustrate the invention in a non limiting fashion.

### EXAMPLE 1

### Inhibition of β-Catenin-mediated Transactivation by Cadherin Derivatives

### Materials and Experimental methods

Plasmid constructions: Full length chicken N-cadherin (Salomon et al. (1992) Extrajunctional distribution of N-cadherin in cultured human endothelial cells. J Cell Sci. 102:7-17, SEQ ID NOs. 1 and 3) was cloned into the pECE expression vector as previously described (Levenberg S. et al. (1998) Long-range and selective autoregulation of cell-cell and cell-matrix adhesions by cadherin or integrin ligands. J. Cell Sci. 111:347-357). The N-cadherin cytoplasmic tail was fused to the IL2Rα extracellular and transmembrane domains in the pcDNA3 expression vector to form the IL2R/N-cadherin chimera. The N-cadherin tail cDNA was isolated from the IL2R/N-cadherin chimera using HindIII and recloned into Bluescript (Stratagene, La Jolla, Calif.). The insert was isolated by EcoRI and XhoI digestion and then ligated, in frame, to a flag epitope in the pECE plasmid. The truncations of the N- and E-cadherin tails were generated by PCR from chicken N-cadherin and mouse E-cadherin (Butz S. (1992) Plakoglobin and β-catenin: distinct but closely related. Science, 257:1142-1144, SEQ ID NOs. 4 and 6) cDNAs by the following PCR conditions: 30 cycles of 15 sec at 94° C., 15 sec at 55° C. and 30 sec at 72° C. The following oligonucleotide primers were used: (a) for the N-cadherin tail amino acid residues 752-912 (SEQ ID NOs. 2 and 3): (5′) 5′-CGGAATTCCAAGCGCCGTGA TAAGGAGCG-3′ (SEQ ID NO. 7) and (3′) 5′-GCTCTAGATCAGTCATAGTC TTGCTCACCAC-3′ (SEQ ID NO. 8); (b) for the N-cadherin C-terminal 71 amino acids, residues 842-912 (SEQ ID NOs. 2 and 3), (5′) 5′-CGGAATTCCATTAATGAGGGACTTAAAGC-3′ (SEQ ID NO. 9) and the same 3′ oligonucleotide as above (i.e., SEQ ID NO. 8); (c) for N-cadherin residues 862-891 (SEQ ID NOs. 2 and 3), (5′) 5′-CGG AATTCCTTAGTCTTTGACTATGAAGG-3′ (SEQ ID NO. 10) and (3′) 5′-GCTCTAGATCAGTCATAGTCTTGCTCACCAC-3′ (SEQ ID NO. 11); (d) for the E-cadherin tail amino acids 735-884 (SEQ ID NOs. 5 and 6), (5′) 5′-CGGAATTCCAGGAGAACGGTGGTCAAAGA-3′ (SEQ ID NO. 12) and (3′) 5′-GCTCTAGACTAGTCGTCCTCGCCACCGC-3′ (SEQ ID NO. 13); (e) for the E-cadherin 72 C-terminal amino acids, residues 813-884 (SEQ ID NOs. 5 and 6), (5′) 5′-CGGAATTCCATCGATGAAAACCTGAA GGC-3′ (SEQ ID NO. 14) and the same 3′ oligonucleotide as above (i.e., SEQ ID NO. 13); (f) for E-cadherin residues 833-862 (SEQ ID NOs. 5 and 6), (5′) 5′-CGGAATTCCTTGGTGTTCGATTACGAGGG-3′ (SEQ ID NO. 15) and (3′) 5′-GCTCTAGATCAATCGTAGTCCTGGTCCTGAT-3′ (SEQ ID NO. 16). The 5′ primers contained EcoRI sites and the 3′ primers XbaI sites. The PCR products were fused to the C-terminus of the green fluorescent protein (GFP) in the pEGFP C1 plasmid (Clontech, Palo Alto, Calif.). HA-LEF-1, mouse β-catenin and TOPFLASH/ FOPFLASH vectors, were kindly provided by Dr. R. Kemler (Huber O. C. (1996) Cadherins and catenins in development. Curr. Opin. Cell Biol. 8:685-691), and Drs. M. van de Wetering and H. Clevers (van de Wetering M. R. et al. (1997) Armadillo coactivates transcription driven by the product of the Drosophila segment polarity gene dTCF. Cell 88:789-799), respectively.

Cell culture and transfections: CHO and SW480 human colon carcinoma cells were cultured in Dulbecco's modified Eagle's medium containing 10% fetal calf serum in an 8% CO₂ incubator at 37° C. Transfections were performed using Lipofectamine (Gibco, BRL, USA) according to the manufacturer's instructions. Stably transfected CHO clones were generated by co-transfection with a plasmid encoding the puromycin resistance gene. Two days after transfection, cells were replated in the presence of 10 μg/ml puromycin (Sigma, Holon, Israel). Stable clones were isolated 2 weeks later and tested for the expression of the transgene by Western blot analysis.

Immunoblotting and immunoprecipitation: The following antibodies were used: polyclonal pan-cadherin or monoclonal anti cadherin (CH-19) and polyclonal anti β-catenin antibodies were from Sigma (Holon, Israel). Monoclonal anti β-catenin, 5H10 (from M. Wheelock), anti IL2Rα (from UBI), anti flag (M2, from IBI), anti HA (clone 12CA5, from Boehringer Mannheim, Germany), anti GFP (polyclonal, from Clontech, Palo Alto, Calif.), anti LEF-1 (polyclonal, kindly provided by Dr. R. Grosschedl). Secondary antibodies for Western blotting were goat anti rabbit or anti mouse conjugated to HRP (Amersham, Buckinghampshire, UK), and for immunofluorescence analysis the secondary antibodies were FITC-goat anti mouse (Cappel, USA) or rhodamine-anti rabbit antibodies (from Jackson Immuno-Research, West Grove, Pa.). Cells were harvested in Laemmli's sample buffer and equal amounts of total cell protein from the different clones, or from transient transfections, were separated by SDS-PAGE, electrotransferred to nitrocellulose and incubated with the different antibodies. For immunoprecipitation, cells were harvested in IP buffer (20 mM Tris pH 8.0, 1% Triton X-100, 140 mM NaCl, 10% glycerol, 1 mM EGTA, 1.5 mM MgCl₂, 1 mM DTT, 1 mM sodium vanadate, and 50 μg/ml PMSF). Equal amounts (500-700 μg) of cell protein were incubated with 1 μl of the relevant antibody for 2 hours at 4° C., followed by incubation with 20 μl of protein A+G/agarose beads (Santa Cruz Biotechnologies, Santa Cruz, Calif.) for an additional 2 hours at 4° C. The agarose beads were washed with 20 mM Tris pH 8.0, 150 mM NaCl, and 0.5% NP-40, and the immune complexes recovered by boiling in Laemmli's sample buffer and resolved by SDS-PAGE.

Triton X-100 fractionation: Cells cultured on 35 mm plates were extracted at room temperature with 200 μl of a buffer containing 0.5% Triton X-100, 2.5 mM EGTA, 5 mM MgCl₂, and 50 mM MES pH 6.0 for 2 min. The Triton-soluble fraction was collected and the plates were washed twice with the same buffer to remove residual soluble material. The insoluble fraction was scraped into 200 μl of this buffer. Equal volumes of the two fractions were examined by SDS-PAGE followed by immunoblotting with anti cadherin and anti β-catenin antibodies.

Immunofluorescence microscopy: Cells cultured on glass coverslips were fixed with 3% paraformaldehyde in phosphate-buffered saline (PBS) and permeabilized with 0.5% Triton X-100. CHO cells were immunofluorescently labeled as described (Levenberg S. et al. (1998) Long-range and selective autoregulation of cell-cell and cell-matrix adhesions by cadherin or integrin ligands. J. Cell Sci. 111:347-357). Nuclei were visualized using DAPI and the fluorescence was examined using a Zeiss Axiophot microscope and a x100/1.3 Planapochromat objective.

Transactivation assays: CHO cells were transfected with TOPFLASH or FOPFLASH vectors (van de Wetering et al., 1997, ibid) and with pCDNA3 coding for β-galactosidase to normalize for transfection efficiency. The transfection mixture also contained either β-catenin, LEF-1 or cadherin derivatives in different combinations, as indicated. SW480 cells were transfected with TOPFLASH and cadherin derivatives. After 48 hours, cells were harvested and tested for luciferase and β-galactosidase activities as follows: Cells were washed 5 times with PBS, resuspended in PM-2 buffer (33 mM NaH₂PO₄, 66 mM Na₂HPO₄, 0.1 mM MnCl₂, 2 mM MgSO₄, 40 mM β-mercaptoethanol) and lysed by 5 cycles of freezing and thawing. Aliquots containing equal amounts of protein were incubated with O-Nitrophenyl-β-D-galactopyranoside (ONPG) at 37° C. until a yellow color appeared. The reactions were stopped with 1 M Na₂CO₃ and the absorbance at 420 nm was determined. Identical aliquots were tested for luciferase activity using luciferine buffer (100 mM Tris-O-Acetate pH 7.8, 10 mM Mg-O-Acetate, 1 mM EDTA, 74 μM luciferine (Boehringer Mannheim, Germany) and 2.22 mM ATP, pH 7.0). The luciferase reaction was monitored by a TD-20e luminometer (Turner, USA) and normalized for the β-galactosidase activity.

### Experimental Results

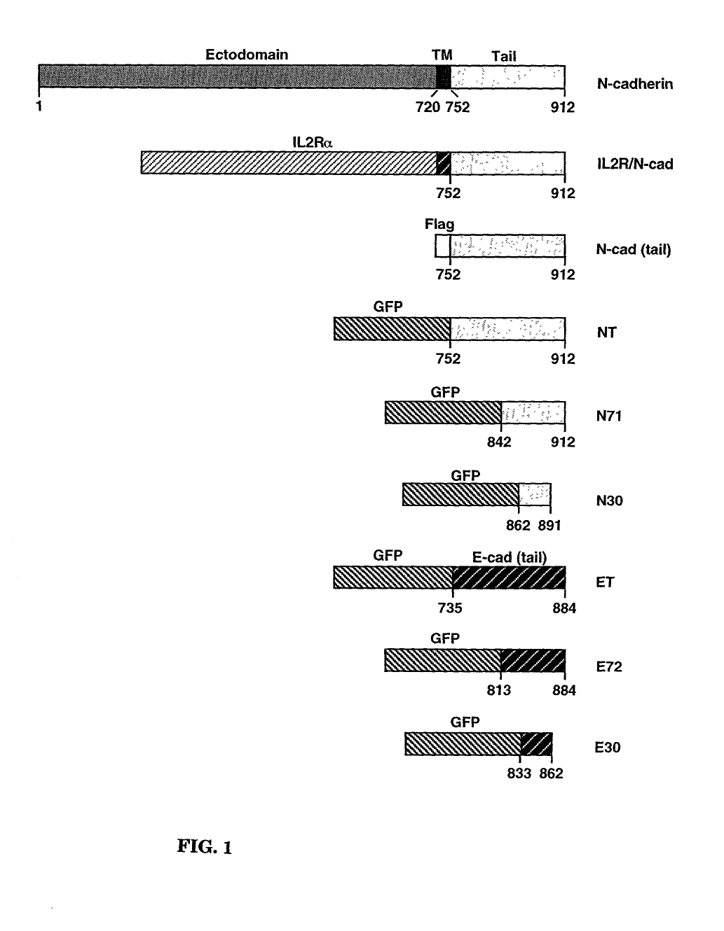

The effects of cadherin derivatives on the stability and localization of β-catenin in CHO cells: To study the effect of cadherin and cadherin derivatives on β-catenin organization and signaling, various cDNA constructs encoding N-cadherin, its cytoplasmic domain fused to the transmembrane and extracellular parts of the interleukin-2 receptor (IL2R), and the soluble cytoplasmic domains of N- and E-cadherin (or parts thereof) were prepared (FIG. 1). Each of the encoded cadherin derivatives contained an antigenic tag (IL2R or flag), or the autofluorescent green fluorescent protein (GFP) that enabled the visualization of the transfected protein.

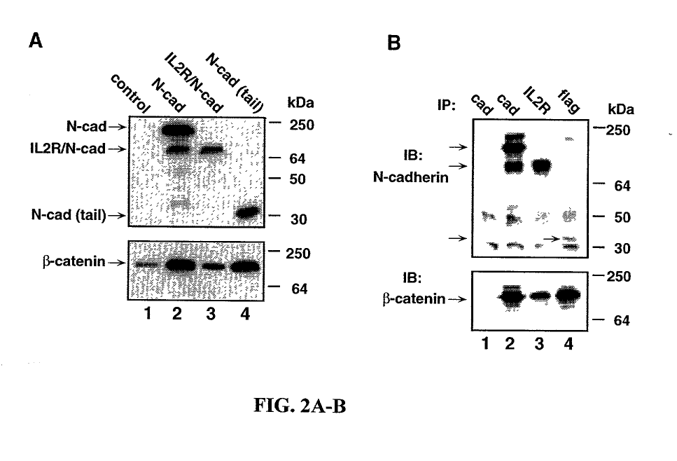

CHO cells that contain very low levels of N-cadherin were stably transfected with either a full length N-cadherin, the IL2R/N-cadherin chimera, or the cytoplasmic tail of N-cadherin. As shown in FIG. 2A, the expression of each of the three cadherin derivatives induced an increase in β-catenin levels, probably by complexing with and protecting β-catenin from degradation. This was supported by showing a direct interaction of these cadherin derivatives with β-catenin by co-immunoprecipitation using antibodies against cadherin, IL2R and flag, followed by immunoblotting with anti β-catenin antibody (FIG. 2B).

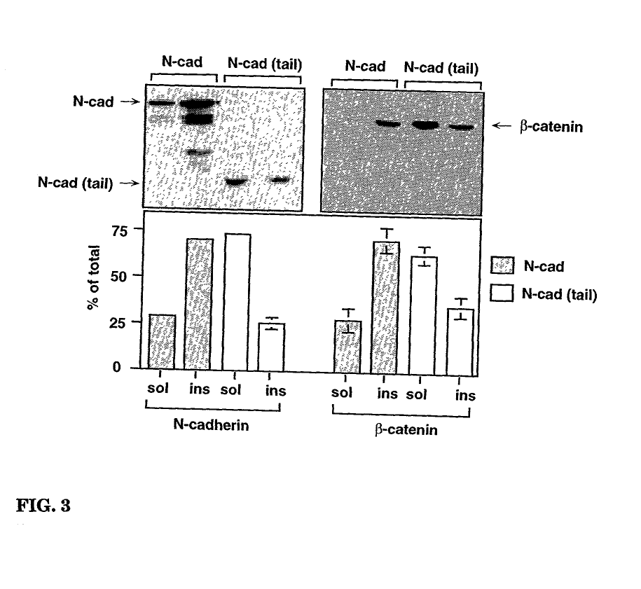

The association of each of the expressed cadherin derivatives with the cytoskeleton was determined by detergent extraction followed by immunoblot analysis, and by immunofluorescence microscopy. Triton X-100 fractionation indicated that 71% of the full length N-cadherin was associated with the Triton X-100-insoluble fraction (FIG. 3). In contrast, 74% of the cadherin tail was Triton-soluble in CHO cells stably expressing these molecules (FIG. 3). Similarly, 72% of β-catenin was detergent-insoluble in CHO cells transfected with N-cadherin, while 64% of β-catenin was Triton-soluble in the N-cadherin tail expressing CHO cells (FIG. 3). The detergent solubility of these molecules in the IL2R/N-cadherin chimera expressing cells was similar to that of cells expressing full length cadherin (results not shown).

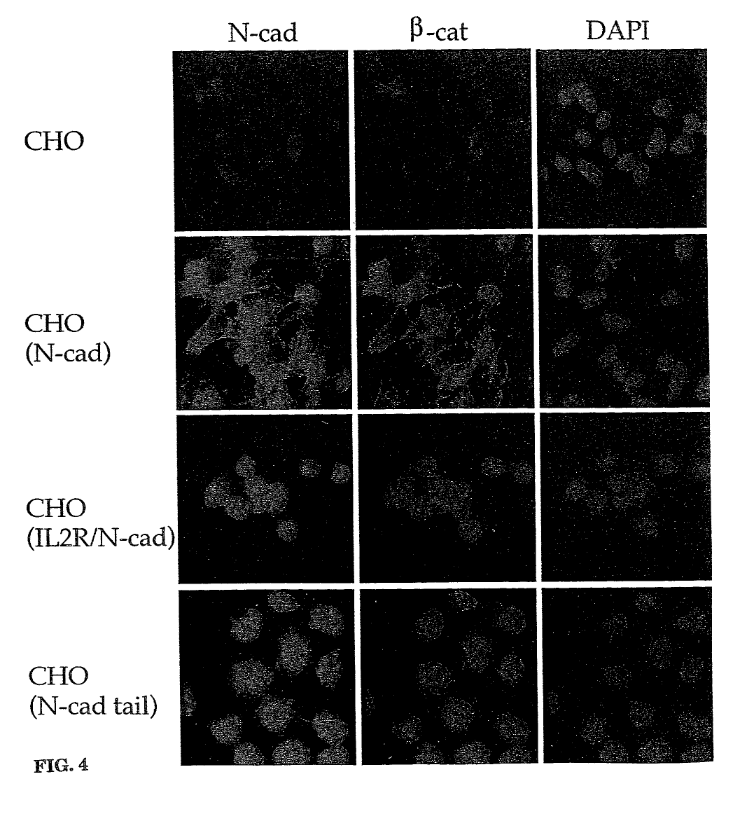

The subcellular localization of the N-cadherin derivatives and of β-catenin was studied by triple fluorescence microscopy. The labeling of the different clones with antibodies to cadherin, β-catenin and with DAPI is shown in FIG. 4. The parental CHO cells express very low levels of both N-cadherin and β-catenin. CHO-N-cadherin cells, on the other hand, exhibited intense N-cadherin and β-catenin staining that was mainly associated with cell-cell junctions. CHO cells expressing the IL2R/N-cadherin chimera displayed a diffuse IL2R/N-cadherin and β-catenin staining over the entire plasma membrane. In contrast, in CHO cells expressing the N-cadherin tail, both the cadherin tail and β-catenin were mainly localized in the nucleus (FIG. 4).

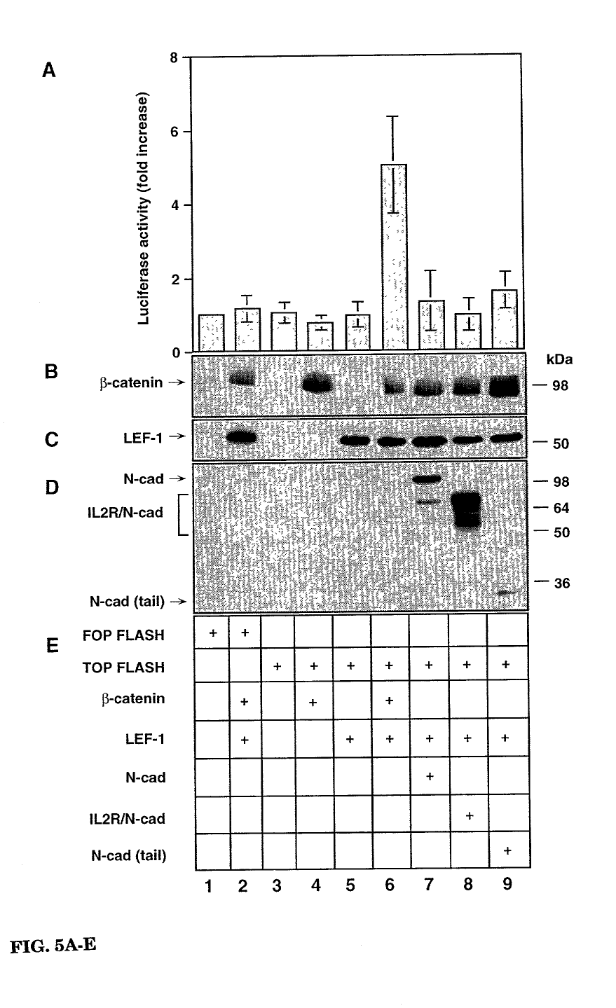

Inhibition of β-catenin-driven transactivation by cadherin derivatives in CHO cells: β-Catenin was shown to associate with transcription factors of the LEF/TCF family, forming a bipartite complex that can transactivate genes containing a LEF/TCF binding sequence near their promoter. In order to study the effect of the different cadherin derivatives on β-catenin-mediated transactivation, constructs were employed containing a multimeric synthetic LEF-1 binding site (TOPFLASH) and, as control, a mutated LEF-1 binding site (FOPFLASH), upstream of a luciferase reporter gene (van de Wetering et al., 1997, ibid). CHO cells were transfected with TOPFLASH or FOPFLASH together with either LEF-1 and β-catenin, or with LEF-1 and each of the cadherin constructs. In all transfections a CMV-β-galactosidase (CMV=cytomegalus virus) plasmid was included as an internal reporter for transfection efficiency. The results presented in FIG. 5A show an about 5-fold increase in luciferase activity after transfection of TOPFLASH together with LEF-1 and β-catenin into CHO cells, compared to TOPFLASH and LEF-1 transfection without β-catenin (FIG. 5A, compare lane 6 to 5). In contrast, transfection of LEF-1 with each of the three cadherin-derived molecules (FIG. 5D, lanes 7-9) was inefficient in elevating luciferase activity (FIG. 5A, lanes 7-9), despite the high levels of β-catenin expression in these cells after transfection (FIG. 5B, lanes 7-9). Moreover, although in N-cadherin tail expressing cells β-catenin was mostly localized in the nucleus (FIG. 4), no specific transactivation was detected in these cells (FIG. 5A, lane 9).

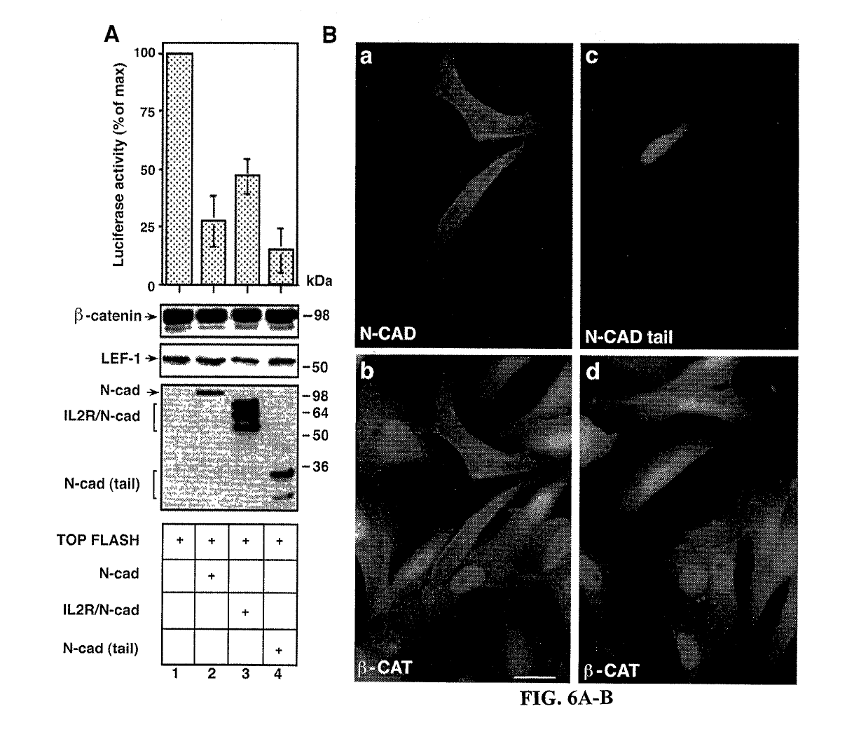

Distribution of N-cadherin derivatives and the effect on β-catenin transactivation in SW480 cells: SW480 colon carcinoma cells express mutant APC, low levels of E-cadherin and relatively high levels of free β-catenin. These cells also display very significant β-catenin-mediated transcription after transfection with TOPFLASH (FIG. 6A, lane 1). To determine the effect of cadherin on this β-catenin-driven transactivation, each of the three N-cadherin derivatives was transfected together with TOPFLASH into SW480 cells and luciferase activity determined. The results shown in FIG. 6A (lanes 2-4) demonstrate that the different cadherin constructs significantly suppressed TOPFLASH-responsive transcription. The most efficient inhibitor of this transactivation was the cadherin tail construct (FIG. 6A, lane 4), which inhibited the reporter gene by more than 85%. Full-length N-cadherin and the IL2R/N-cadherin chimera decreased the activity of the reporter by about 70% and 50%, respectively. Immunofluorescence staining of SW480 cells transfected with N-cadherin (FIG. 6B, N-CAD) showed that N-cadherin transfection resulted in binding of the endogenous β-catenin to the plasma membrane (FIG. 6B, β-CAT left), similarly to the results obtained with IL2R/N-cadherin chimera (data not shown), while the transfected cytoplasmic tail of N-cadherin (FIG. 6B, N-CAD tail) colocalized with β-catenin in the nucleus (FIG. 6B, β-CAT right).

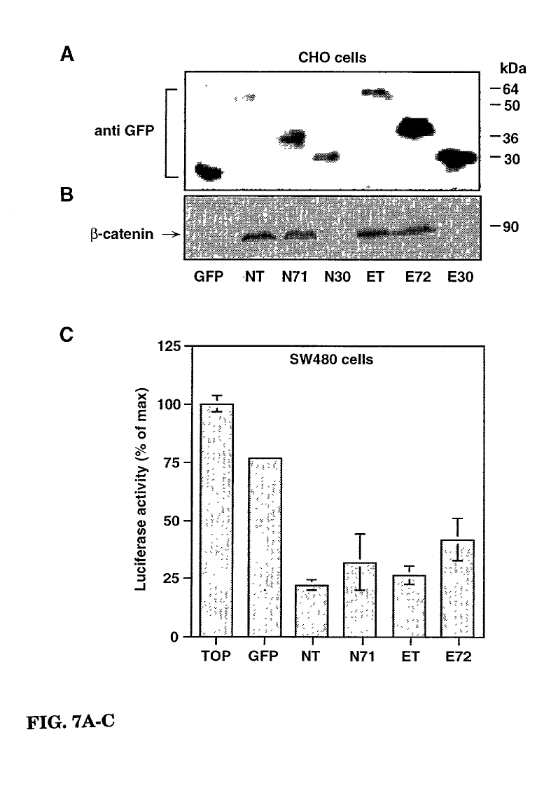

Identification of the region in the C-terminal tail of cadherin that stabilizes β-catenin and inhibits its transactivation potential. To identify the region in the cadherin tail that is involved in the suppression of LEF-1-responsive transcription, the inhibitory activity of the cytoplasmic tails of E- and N-cadherin, and that of two deletion mutants prepared from these tails that were fused to GFP were compared (FIG. 1). The constructs were transfected into CHO cells to determine the level of β-catenin, and into SW480 cells to examine their inhibitory activity on transactivation (FIG. 7).

Western blot analysis of the GFP-cadherin constructs expressed in CHO cells is shown in FIG. 7A. The migration of the expressed proteins analyzed by SDS-PAGE was in agreement with the expected molecular weights for these constructs. Immunoblotting of the same extracts with anti β-catenin antibody indicated that the N- (NT) and E-cadherin (ET) tails were both efficient in stabilizing β-catenin against degradation (FIG. 7B). Furthermore, the C-terminal 71 amino acids of N-cadherin (N71, amino acids 842-912, SEQ ID NOs. 2 and 3, FIG. 1) and the C-terminal 72 amino acids of E-cadherin (E72 amino acids 813-884, SEQ ID NOs. 5 and 6, FIG. 1) could also stabilize β-catenin in CHO cells (FIG. 7B). In contrast, shorter fragments of the cadherin cytoplasmic tails consisting of about 30 amino acids of the N- (N30, amino acids 860-891, SEQ ID NOs. 1 and 3, FIG. 1) and E-cadherin tails (E30, amino acids 833-862, SEQ ID NOs. 5 and 6, FIG. 1), corresponding to the middle part of N71 and E72, were ineffective in stabilizing β-catenin against degradation (FIG. 7B).

Analysis of transactivation in SW480 cells (FIG. 7C) showed that the GFP-constructs containing the N- and E-cadherin tails inhibited luciferase activity by 80% and 75% respectively, while GFP only slightly reduced this activity (FIG. 7C). The shorter GFP-N71 and GFP-E72 constructs were somewhat less efficient and reduced transactivation by 70% and 60% respectively. The 30 amino acid E- and N-cadherin tail fragments did not affect β-catenin-mediated transactivation in SW480 cells (data not shown).

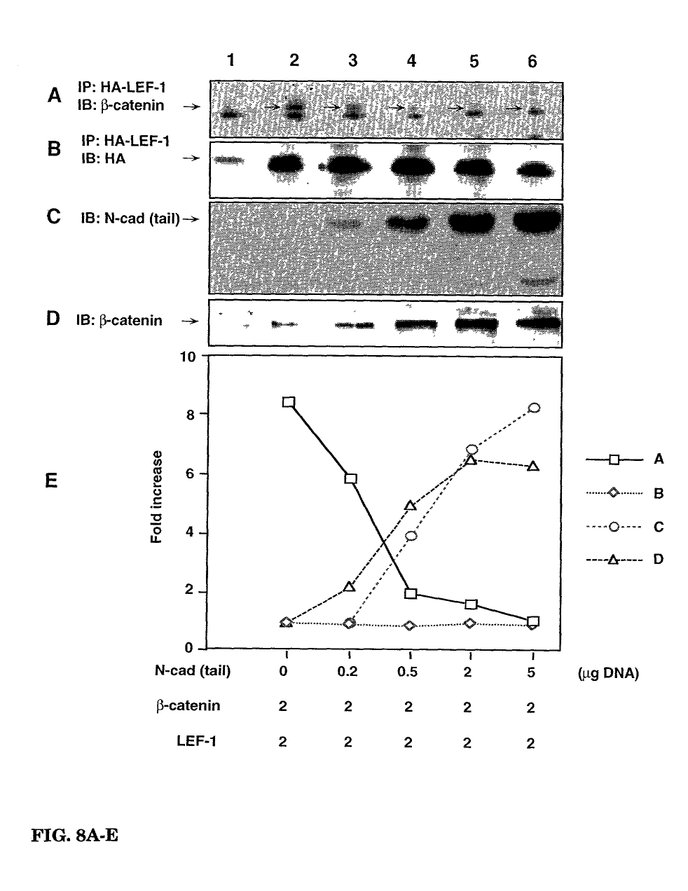

The N-cadherin tail inhibits the formation of a LEF-1-β-catenin complex: The inhibition by N-cadherin of β-catenin driven-transactivation could result from either displacement of LEF-1 binding to β-catenin by N-cadherin, or by formation of a ternary complex (cadherin-LEF-1-β-catenin) that has no transactivation potential. To distinguish between these possibilities, CHO cells were transfected with constant amounts of HA-tagged LEF-1 and with β-catenin, and increasing amounts of a cDNA encoding the N-cadherin tail (FIG. 8E, lanes 2-6). After transfection, LEF-1 was immunoprecipitated with anti HA antibody and the associated β-catenin was detected by Western blot analysis (FIG. 8A). The results shown in FIGS. 8A and 8E demonstrate that the increase in the level of N-cadherin tail inhibited β-catenin binding to LEF-1 in a dose dependent manner. This effect was especially prominent considering that the total amount of β-catenin in the transfected cells increased (FIG. 8D, compare lanes 2 to 6) in parallel with the level of the transfected N-cadherin tail (FIG. 8C, lanes 2-6), due to stabilization of β-catenin by its binding to the cadherin tail. This implies that the N-cadherin tail is effective in competing with LEF-1/β-catenin complex formation in the nucleus.

### EXAMPLE 2

### Differential Nuclear Translocation and Transactivation Potential of β-Catenin and Plakoglobin

### Materials and experimental Methods

Cell Culture and Transfections: Canine kidney epithelial cells MDCK, human fibrosarcoma HT1080, 293-T human embryonic kidney cells, Balb/C mouse 3T3 and human colon carcinoma SW480 cell lines were cultured in Dulbecco's modified Eagle's medium supplemented with 10% calf serum (Gibco, Grand Island, N.Y.) at 37° C. in the presence of 7% CO₂. The human renal carcinoma cell line KTCTL60 was grown in RPMI medium and 10% calf serum. Cells were transiently transfected with the cDNA constructs described below, using Ca²⁺-phosphate or lipofectamine (Gibco) and the expression of the transgene was assessed between 24 and 48 hours after transfection. In some experiments, the expression of the stably transfected NH₂ terminus-deleted β-catenin (Δ57) was enhanced in HT1080 cells by overnight treatment with 2 mM sodium butyrate.

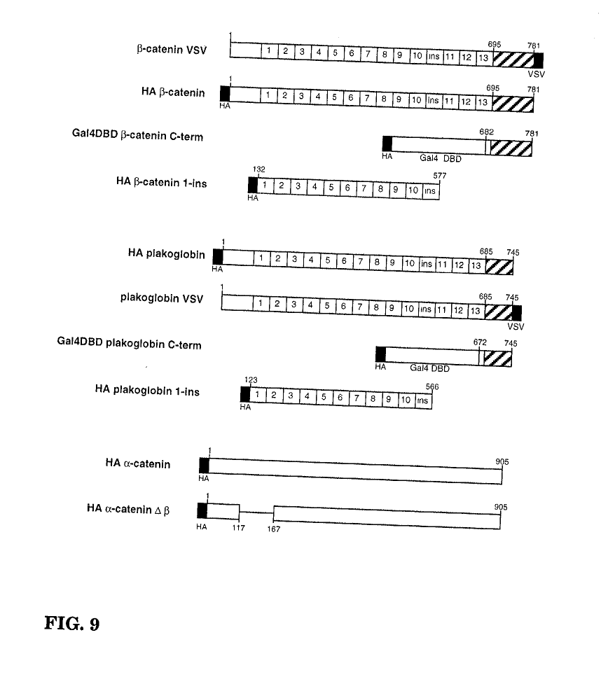

Construction of Plasmids: The β-catenin, plakoglobin and α-catenin (SEQ ID NOs. 17 and 19) constructs were cloned in frame into the pCGN expression vector containing a 16-amino acids hemaglutinin (HA)-tag at the NH₂-terminus (FIG. 9). HA-β-catenin was obtained by PCR amplification of the 5′ end of mouse β-catenin using oligonucleotides 5′-ACCTTCTAGAATGGCTACTCA AGCTGACCTG-3′ (SEQ ID NO. 20) and 5′-ATGAGCAGCGTCAAACT GCG-3′ (SEQ ID NO. 21). The XbaI/SphI fragment of the PCR product and the SphI/BamHI fragment of mouse β-catenin were joined and cloned into the pCGN vector. A β-catenin mutant containing armadillo repeats 1 to 10 (HA β-catenin 1-ins, FIG. 9) was obtained using oligonucleotides 5′-A CCTTCTAGATTGAAACATGCAGTTGTCAATTTG-3′ (SEQ ID NO. 22) and 5′-ACCTGGATCCAGCTCCAGTACACCCTTCG-3′ (SEQ ID NO. 23). The amplified fragment was cloned into pCGN as an XbaI/BamHI fragment. VSV-tagged β-catenin at the COOH terminus was obtained by PCR amplification of the 3′ end of Xenopus β-catenin using 5′-AACTGCTCCTCTTACTGA-3′ (SEQ ID NO. 24) and 5′-TATCC CGGGTCAAGTCAGTGTCAAACCA-3′ (SEQ ID NO. 25). An XbaI/SmaI fragment of the amplified product was joined to an XbaI/EcoRI digest of Xenopus β-catenin from Bluescript, and cloned into the pSY-1 plasmid containing an 11 amino acids VSV G-protein tag at the COOH terminus. The product was subcloned into pCI-neo (Promega, Madison, Wis.).

HA-tagged human plakoglobin (HA plakoglobin, FIG. 9) was obtained by PCR-amplification of the 5′ end of human plakoglobin cDNA (pHPG 5.1) using 5′-ACCTTCTAGAATGGAGGTGA TGAACCTGATGG-3′ (SEQ ID NO. 26) and 5′-AGCTGAGCATGCGGAC CAGAGC-3′ (SEQ ID NO. 27) oligonucleotide primers. The XbaI/SphI fragment of the PCR product and the SphI/BamHI fragment of human plakoglobin cDNA were cloned into the pCGN vector. A plakoglobin mutant containing armadillo repeats 1 to 10 (HA plakoglobin 1-ins, FIG. 9) was amplified using 5′-ACCTTCTAGACTCAAGTCGGCCATTGTGC-3′ (SEQ ID NO. 28) and 5′-ACCTGGATCCTGCTCCGGTGCAGCCCTC C-3′ (SEQ ID NO. 29) oligonucleotides. The amplified fragment was cloned into the XbaI/BamHI site of pCGN. A COOH-terminus VSV-tagged plakoglobin (plakoglobin-VSV, FIG. 9) was obtained by amplifying the 3′ terminus of plakoglobin cDNA using 5′-AGGCCGCC CGGGCAGCATG-3′ (SEQ ID NO. 30) and 5′-CGCATGGAGATCTT CCGGCTC-3′ (SEQ ID NO. 31) oligonucleotide primers. The EcoRI/BgIII fragment of the plakoglobin cDNA and the BgIII/SmaI fragment of the amplified product were cloned in frame with the COOH-terminus VSV-tag into the pSY-1 plasmid. VSV-tagged plakoglobin was recloned into the pCI-neo expression vector (Promega, Madison, Wis.) as an EcoRI/NotI fragment.

HA-tagged α-catenin (HA α-catenin, FIG. 9) was constructed by amplification of the 5′ end of chicken α-catenin (SEQ ID NOs. 17 and 19) using 5′-ACCTTCTAGAATGACGGCTGTTACTG CAGG-3′ (SEQ ID NO. 32) and 5′-GCCTTCTTAGAGCGCCCTCG-3′ (SEQ ID NO. 33) oligonucleotide primers. The XbaI/ApaI fragment of the PCR product and the ApaI/KpnI fragment of chicken α-catenin from pLK-α-catenin were cloned into the pCGN vector. A HA-tagged mutant α-catenin (HAα-catenin Δβ, FIG. 9) lacking the β-catenin binding site (amino acids 118-166) was obtained by PCR-based mutagenesis using two fragments from the 5′ end of α-catenin corresponding to amino acids 1-117 (SEQ ID NOs. 18 and 19) and 167-303 (SEQ ID NOs. 18 and 19), and oligonucleotides 5′-ACCTTCTAGAATGACGGCTGTTACTGCAGG-3′ (SEQ ID NO. 34), 5′-TGCCAGCGGAGCAGGGGTCATCAGCAAAC-3′ (SEQ ID NO. 35), 5′-CTGCTCCGCTGGCACCGAGCAGGATCTG-3′ (SEQ ID NO. 36) and 5′-ACGTTGCTCACTGAAGGTCG-3′ (SEQ ID NO. 37). The two fragments were joined, and the product was amplified using 5′-ACCTTCTAGAATGACGGCTGTTACTGCAGG-3′ (SEQ ID NO. 38) and 5′-ACGTTGCTCACTGAAGGTCG-3′ (SEQ ID NO. 39) primers. The XbaI/BamHI fragment of the mutant construct and the BamHI/KpnI fragment of chicken α-catenin from HA-α-catenin were cloned into the pCGN vector.

HA-tagged LEF-1 at the COOH terminus, was a generous gift from Dr. Rolf Kemler (Max Planck Institute, Freiburg, Germany).

The DNA-binding domain of Gal4 (Gal4DBD) was obtained by PCR using a 5′-ACCTTCTAGAATGAAGCTACTGTCTTCTATC-3′ (SEQ ID NO. 40) oligonucleotide with an XbaI site, and 5′-ACCTGAGCTCCGAT ACAGTCAACTGTCTTTG-3′ (SEQ ID NO. 41) with a SacI site (for Gal4DBD β-catenin and Gal4DBD plakoglobin, FIG. 9), and with the antisense primer 5′-ACCTGGATCCTACGATACAGTCAACTGTCTTG-3′ (SEQ ID NO. 42) creating a BaMHI site (for Gal4DBD, FIG. 9). Fragments corresponding to the COOH terminus of β-catenin (amino acids 682-781, Butz et al., 1994, ibid) and plakoglobin (amino acids 672-745, Franke, W. W. et al. (1989) Molecular cloning and amino acid sequence of human plakoglobin, the common junctional plaque protein. Proc. Natl. Acad. Sci. USA. 86:4027-4031) were obtained by PCR using sense primers containing a SacI site and antisense primers containing a BamHI site. The XbaI/SacI fragment of Gal4DBD and the SacI/BamHI fragments of β-catenin and plakoglobin were joined and cloned into pCGN (FIG. 9). The XbaI/BamHI fragment of Gal4DBD was cloned into pCGN and used as control in transactivation assays. A CMV-promoter driven N-cadherin cDNA was used (Salomon et al, 1992, ibid). The validity of the constructs shown in FIG. 9 was verified by sequencing, and the size of the proteins determined after transfection into 293-T and MDCK cells by Western blotting with anti HA and anti VSV antibodies.

Transactivation Assays: Transactivation assays were conducted with SW480 and 293-T cells grown in 35 mm/diameter dishes that were transfected with 0.5 μg of a plasmid containing a multimeric LEF-1 consensus binding sequence driving the luciferase reporter gene (TOPFLASH), or a mutant inactive form (FOPFLASH, generously provided by Drs. H. Clevers and M. van de Wetering Univ. Utrecht, The Netherlands). A plasmid encoding β-galactosidase (0.5 μg) was cotransfected to enable normalization for transfection efficiency. The relevant plasmid expressing catenin constructs (4.5 μg) was co-transfected with the reporter, or an empty expression vector was included. After 24-48 hours expression of the reporter (luciferase) and the control (β-galactosidase) genes were determined using enzyme assay systems from Promega (Madison, Wis.).

The Gal4RE reporter plasmid for determining transactivation by Gal4DBD chimeras was constructed as follows: oligonucleotides comprising a dimer of 17 nucleotides of the Gal4 binding sequences containing a SacI site at the 5′ end and a BgIII site at the 3′ end, were obtained by PCR amplification using 5′-GGAAGACTCTC CTCCGGATCCGGAAGACTCTCCTCC-3′ (SEQ ID NO. 43) and 5′-GAT CGGAGGAGAGTCTTCCGGATCCGGAGGAGAGTCTT CCAGCT-3′ (SEQ ID NO. 44) and subcloned as a SacI/BgIII fragment into the pGL3-promoter plasmid (Promega, Madison, Wis.) driving luciferase expression.

Northern blot hybridization: Total RNA was extracted from cells by the guanidinium thiocyanate method. Northern blots containing 20 μg per lane of total RNA were stained with methylene blue to determine the positions of 18S and 28S rRNA markers, and then hybridized with plakoglobin (Franke et al, 1989, ibid) and β-catenin (Butz et al., 1992, ibid) cDNAs, which were labeled with ³²P-dCTP by the random priming technique.

Protease Inhibitors: The calpain inhibitor N-Acetyl-Leu-Leu-Norleucinal (ALLN, used at 25 μM) and the inactive analog N-Acetyl-Leu-Leu-Normethional (ALLM, used at 10 μg/ml) were purchased from Sigma (St. Louis, Mo.). Lactacystin A (dissolved in water at 0.4 mg/ml was used at a final concentration of 4 μg/ml) and MG-132 (used at 10 or 20 μM) were purchased from Calbiochem (La Jolla, Calif.).

Immunofluorescence Microscopy: Cells cultured on glass coverslips were fixed with 3.7% paraformaldehyde in PBS and permeabilized with 0.5% Triton X-100. Monoclonal antibodies against human plakoglobin (11E4), β-catenin (5H10), the COOH terminus of β-catenin (6F9) and α-catenin (1G5) were described and kindly provided by Drs. M. Wheelock and K. Johnson (Univ. Toledo, Ohio). The secondary antibody was FITC- or Cy3-labeled goat anti mouse IgG (Jackson ImmunoResearch Laboratories, West Grove, Pa.). Polyclonal antiserum against β-catenin, monoclonal antibody against pan cadherin (CH-19), vinculin (hours-VIN 1) and α-actinin (BM75.2) were from Sigma (Holon, Israel). Monoclonal antibodies against the splicing factor SC35 and the HA epitope were kindly provided by Dr. D. Helfman (CSH Laboratory, N.Y.). Polyclonal rabbit antibody against the VSV-G epitope was a gift from Dr. JC Perriard (ETH, Zurich, Switzerland). FITC-labeled goat anti-rabbit IgG antibody was from Cappel/ICN. Monoclonal antibody against LEF-1 was kindly provided by Dr. R. Grosschedl (Univ. Calif. San Francisco, Calif.). Polyclonal anti HA-tag antibody was a gift from Dr. M. Oren (Weizmann Institute, Israel). Antibodies to actin were provided by Dr. J. Lessard (Childrens Hospital Res. Foundation, Cincinnati, Ohio) and Dr. I. Herman (Tufts Univ. Boston, Mass.). The cells were examined by epifluorescence with a Zeiss Axiophot microscope. To determine the level of overexpression relative to the endogenous protein, digitized immunofluorecent microscopy was employed. Images of the fluorescent cells were recorded with a cooled, scientific grade CCD camera (Photometrics, Tucson, Ariz.). Integrated fluorescent intensities in transfected and nontransfected cells were determined, after the background was subtracted.

Electron Microscopy: Cells were processed for conventional electronmicroscopy by fixation with 2% glutaraldehyde followed by 1% OsO₄. The samples were dehydrated and embedded in Epon, sectioned, and examined in a EM410 Philips electronmicroscope. Samples processed for immunoelectronnmicroscopy were fixed with 3% paraformaldehyde and 0.1% glutaraldehyde in 100 mM cacodylate buffer (pH 7.4) containing 5 mM CaCl₂, embedded in 10% gelatin and re-fixed as above, incubated with sucrose, frozen, and cryosectioned. The sections were incubated with monoclonal anti vinculin (hours-VIN-1), monoclonal anti β-catenin (5H10), or polyclonal anti VSV-tag (to recognize the VSV-tagged β-catenin), followed by secondary antibody conjugated to 10 nm gold particles (Zymed, San Francisco, Calif.). The sections were embedded in methyl cellulose and examined in a Philips CM12 electron microscope.

Polyacrylamide Gel Electrophoresis and Immunoblotting: Equal amounts of total cell protein were separated by SDS PAGE, electro-transferred to nitrocellulose and incubated with monoclonal antibodies. The antigens were visualized by the enhanced chemiluminescence (Amersham, Buckinghampshire, U.K). In some experiments, cells were fractionated into Triton X-100-soluble and -insoluble fractions. Briefly, cells cultured on 35 mm dishes were incubated in 0.5 ml buffer containing 50 mM MES, pH 6.8, 2.5 mM EGTA, 5 mM MgCl₂ and 0.5% Triton-X-100, at room temperature for 3 minutes. The Triton X-100-soluble fraction was removed, and the insoluble fraction was scraped into 0.5 ml of the same buffer. Equal volumes of the two fractions were analyzed by SDS PAGE followed by immunoblotting with the various antibodies.

### Experimental Results

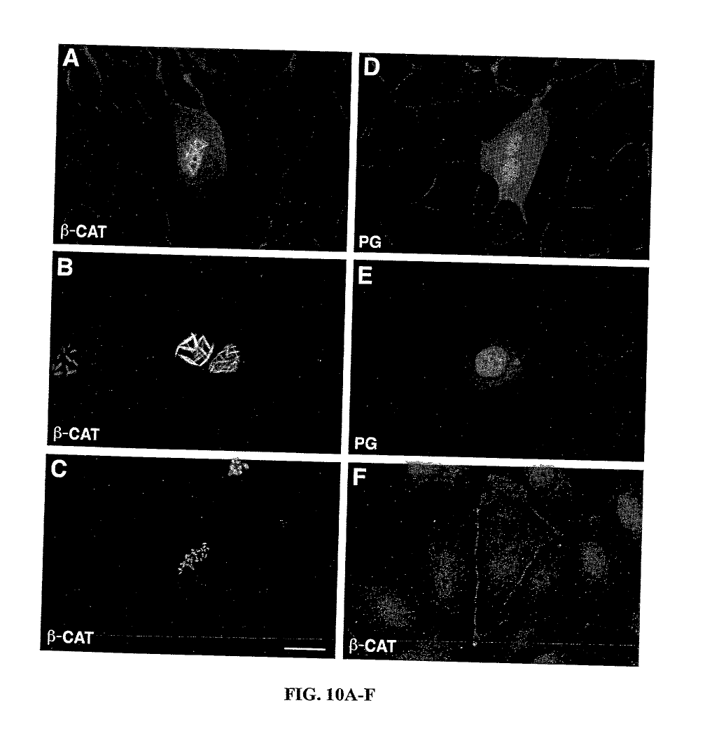

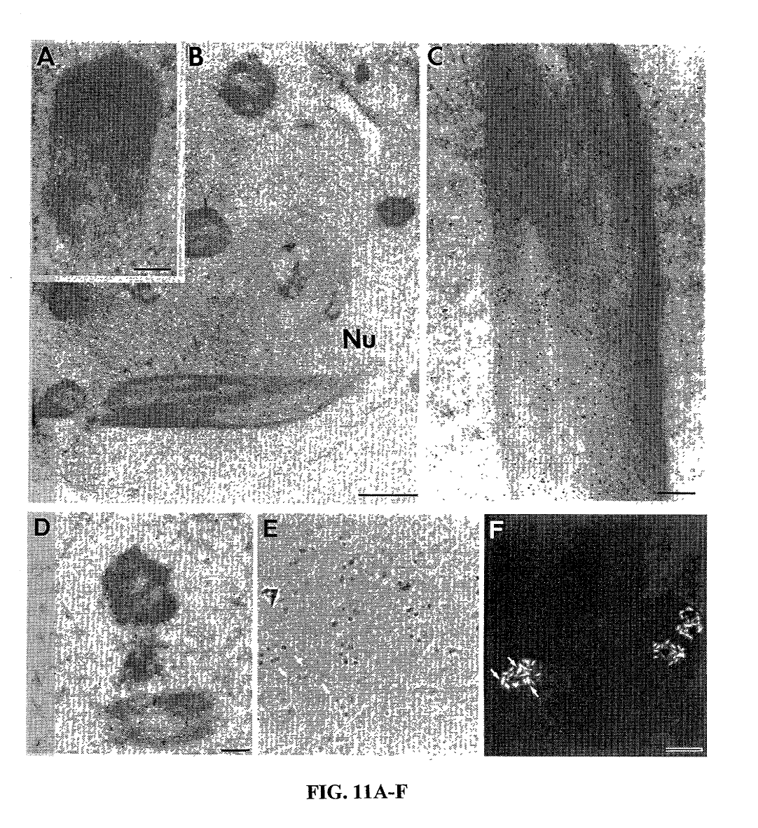

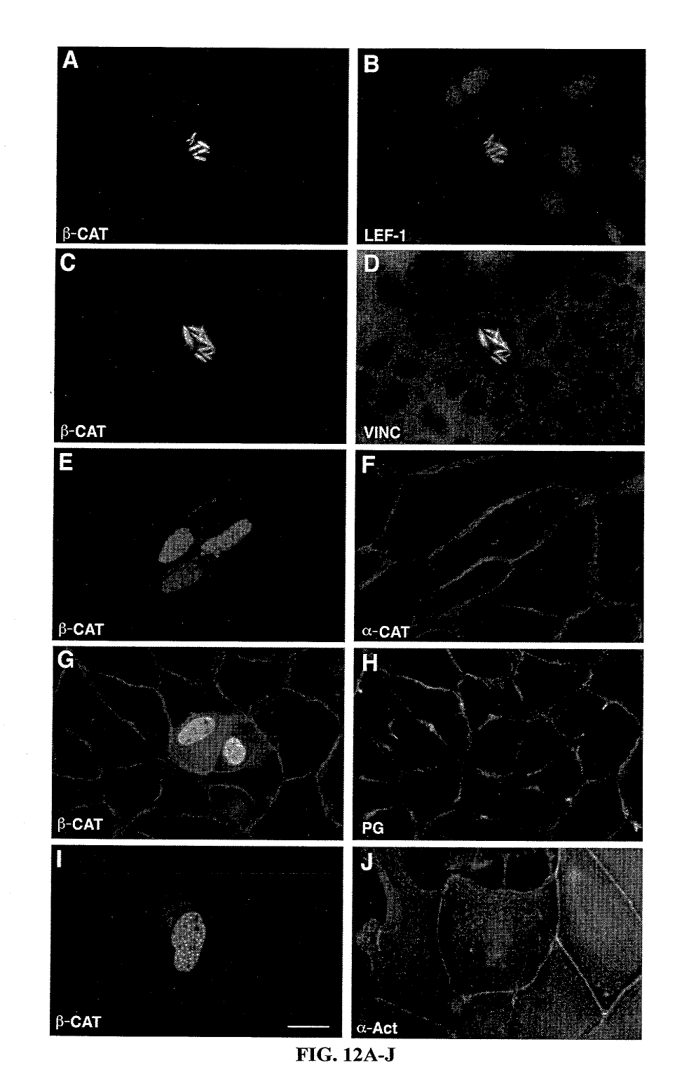

Overexpression of β-Catenin and Plakoglobin in MDCK Cells Results in their Nuclear Accumulation and in Nuclear Translocation of Vinculin: To determine the localization of overexpressed β-catenin and plakoglobin, MDCK cells that normally display these molecules at cell-cell junctions, were transiently transfected with VSV-tagged-β-catenin or plakoglobin cDNA constructs (FIG. 9) and immunostained with either anti VSV, anti β-catenin, or anti plakoglobin antibodies. The results in FIG. 10 show that when expressed at very low level, β-catenin was detected at cell-cell junctions (FIG. 10F), but in cells expressing higher levels (about 5 fold over the endogenous protein level, as determined by digital immunofluorescent microscopy), most of the transfected molecules were localized in the nuclei of cells, either in a diffuse form, or in aggregates of various shapes (speckles and rods, FIGS. 10A-C). These β-catenin-containing aggregates were organized in discernible structures that could be identified by phase microscopy (FIGS. 11E and 11F). Transmission electron microscopy of Epon-embedded cells revealed within the nucleus highly ordered bodies consisting of laterally aligned filamentous structures, with a filament diameter of about 10 nm and packing density of about 50 filaments per μm (FIG. 11A). Immunogold labeling of ultrathin frozen sections indicated that these intranuclear bodies contained high levels of β-catenin (FIGS. 11B and 11C).

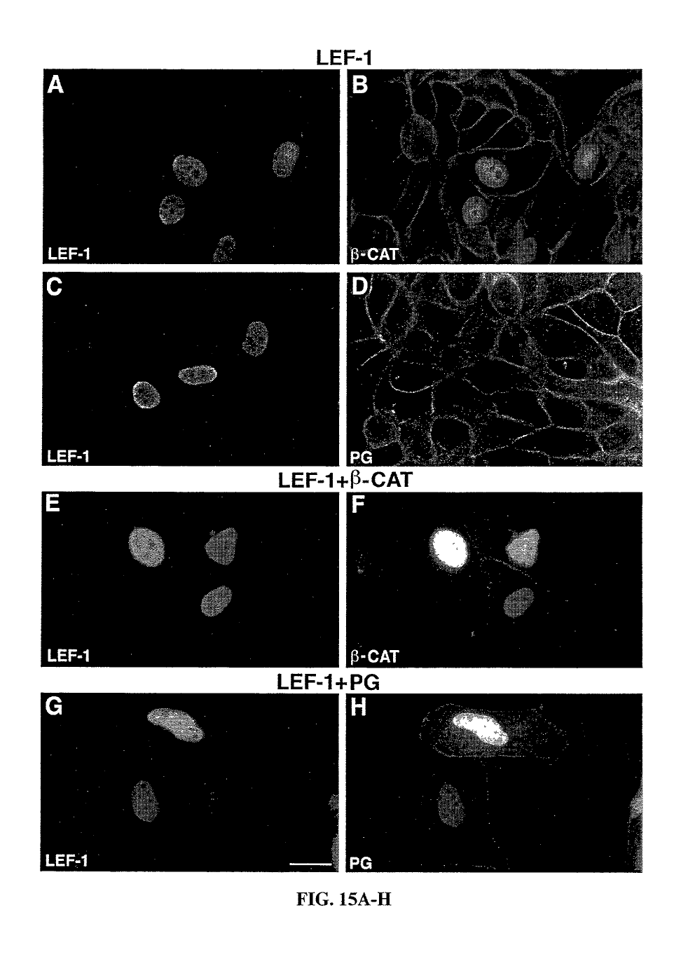

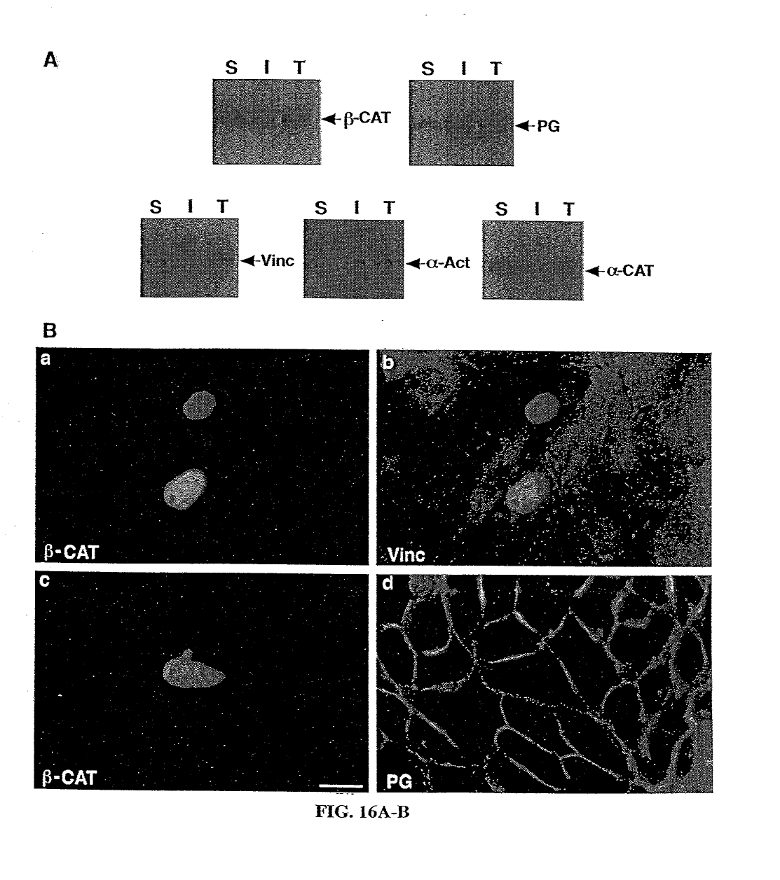

Transfection of plakoglobin also resulted in nuclear accumulation of the molecule and in addition showed diffuse cytoplasmic (FIG. 10D) and junctional staining (see below, FIG. 15H). While nuclear aggregates were observed in plakoglobin-transfected cells (FIG. 10E), large rods were not detected in the nuclei of these cells. Similar structures were observed with HA-tagged and untagged β-catenin or plakoglobin (data not shown), suggesting that these structures in the nucleus assembled due to high levels of these proteins and are not attributable to tagging.

The unique assembly of β-catenin into discrete nuclear structures is, most probably non physiological, yet it enabled us to examine the association of other molecules with β-catenin (FIGS. 12A-J). Interestingly, in addition to the transcription factor LEF-1 (FIG. 12B) that was shown to complex with β-catenin in the nucleus, vinculin also strongly associated with the β-catenin-containing speckles and rods in the nucleus (FIGS. 12C and 12D). This was also confirmed by immunogold labeling of ultrathin frozen sections with anti vinculin antibodies showing that the labeling was distributed throughout the entire nuclear aggregate (FIG. 11D). In contrast, other endogenous proteins known to be involved in linking cadherins to actin at adherens junctions such as α-catenin (FIG. 12F), α-actinin (FIG. 12J) and plakoglobin (FIG. 12H) were not associated with the β-catenin-containing nuclear aggregates. Actin was also missing from these nuclear aggregates (results not shown). Furthermore, the β-catenin-containing rods and speckles were clearly distinct from other nuclear structures such as the splicing component SC35 (data not shown), which also displays a speckled nuclear organization in many cells.

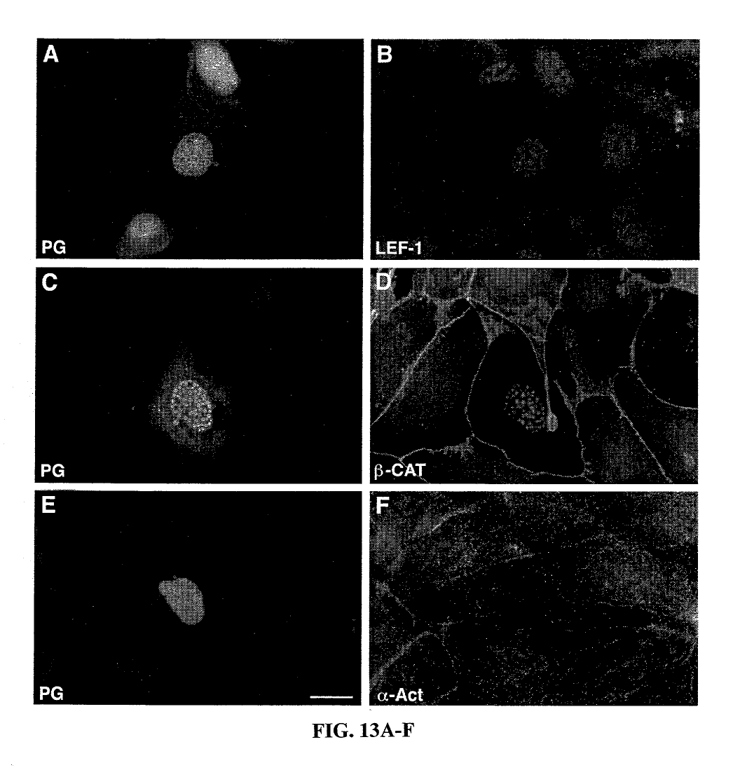

The molecular interactions of plakoglobin were distinctly different from those formed by β-catenin. While nuclear speckles in MDCK cells overexpressing plakoglobin displayed some faint staining for LEF-1 (FIG. 13B compare to 13A), this was less pronounced than that seen with β-catenin (FIG. 12B), and essentially no nuclear co-staining for vinculin, α-catenin (not shown), or α-actinin (FIG. 13F) was observed. Interestingly, plakoglobin-containing nuclear speckles (FIG. 13C) were also stained with anti β-catenin, and the cytoplasm of these cells was essentially devoid of the diffuse β-catenin staining seen in non-transfected cells (FIG. 13D). This may be explained by the capacity of plakoglobin to compete and release β-catenin from its other partners (i.e., cadherin or APC) leading to its nuclear translocation.

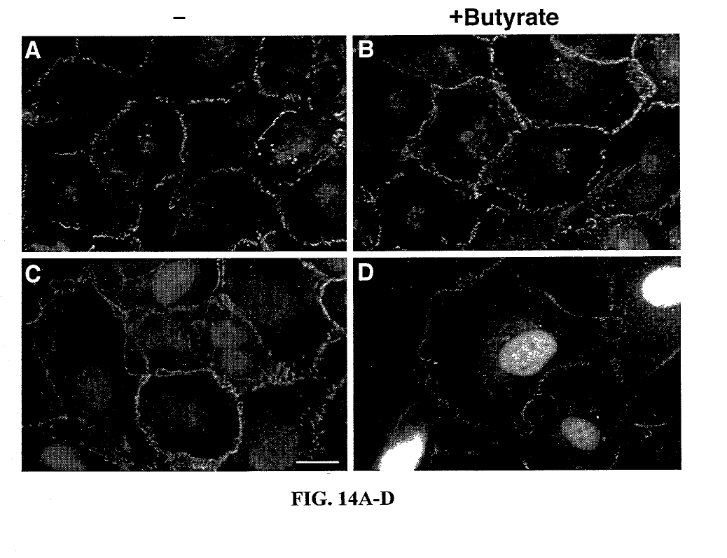

Nuclear Accumulation of β-Catenin After Induced Overexpression: Transient transfection usually results in very high and non physiological levels of expression (and organization) of the transfected molecules. To obtain information on β-catenin that is more physiologically relevant, HT1080 cells, were isolated, stably expressing a mutant β-catenin molecule lacking the NH₂-terminal 57 amino acids (ΔN57) that is considerably more stable than the wt protein. In such stably transfected cells the level of expression was low, and only faint nuclear β-catenin staining was detected, with the majority of β-catenin localized at cell-cell junctions (FIG. 14C). However, when the cells were treated with butyrate, an about 2 fold increase in the expression of the transgene was observed by western blot analysis (data not shown), and a dramatic translocation of β-catenin into the nucleus occurred (FIG. 14D). This translocation was not observed in butyrate-treated control neo^{r} HT1080 cells (FIG. 14A, and B). These results suggest that an increase in β-catenin over certain threshold levels of either transiently or inducibly expressed β-catenin results in its nuclear translocation and accumulation.

LEF-1 Overexpression Induces Translocation of Endogenous β-Catenin but not Plakoglobin into the Nuclei of MDCK Cells: Nuclear translocation of β-catenin was shown to be promoted by elevated LEF-1 expression. The ability of transfected LEF-1 to induce the translocation of endogenous β-catenin and plakoglobin into the nuclei of MDCK cells were thus compared. Cells were transfected with a HA-tagged LEF-1 and after 36 hours doubly immunostained for LEF-1 and β-catenin, or for LEF-1 and plakoglobin. As shown in FIGS. 15A-H, while endogenous β-catenin was efficiently translocated into the nucleus in LEF-1-transfected cells and colocalized with LEF-1 (FIG. 15A, and B), plakoglobin was not similarly translocated into the nucleus (FIG. 15D compare to C). This difference between β-catenin and plakoglobin could result from the larger pool of diffuse β-catenin (FIG. 16A) that is available for complexing and translocation into the nucleus with LEF-1. In contrast, plakoglobin was almost exclusively found in the Triton X-100-insoluble fraction (FIG. 16A), in association with adherens junctions and desmosomes. Vinculin and α-catenin that also display a large Triton X-100-soluble fraction (FIG. 16A), in contrast to α-actinin and plakoglobin, were not translocated into the nucleus after LEF-1 transfection (data not shown).

When LEF-1 was overexpressed in MDCK cells together with plakoglobin, both proteins were localized in the nucleus and displayed diffuse staining (FIGS. 15G and H), similar to β-catenin (FIGS. 15E and F). In cells doubly transfected with β-catenin and LEF-1, vinculin was also translocated into the nucleus displaying diffuse staining (FIG. 16B, β-CAT (upper inset) and Vinc), while plakoglobin was not detected in the nuclei of such cells (FIG. 16B, β-CAT (lower inset) and PG). Since LEF-1 transfection did not result in nuclear localization of vinculin (data not shown), this implies that vinculin translocation into the nucleus is only related to that of β-catenin.

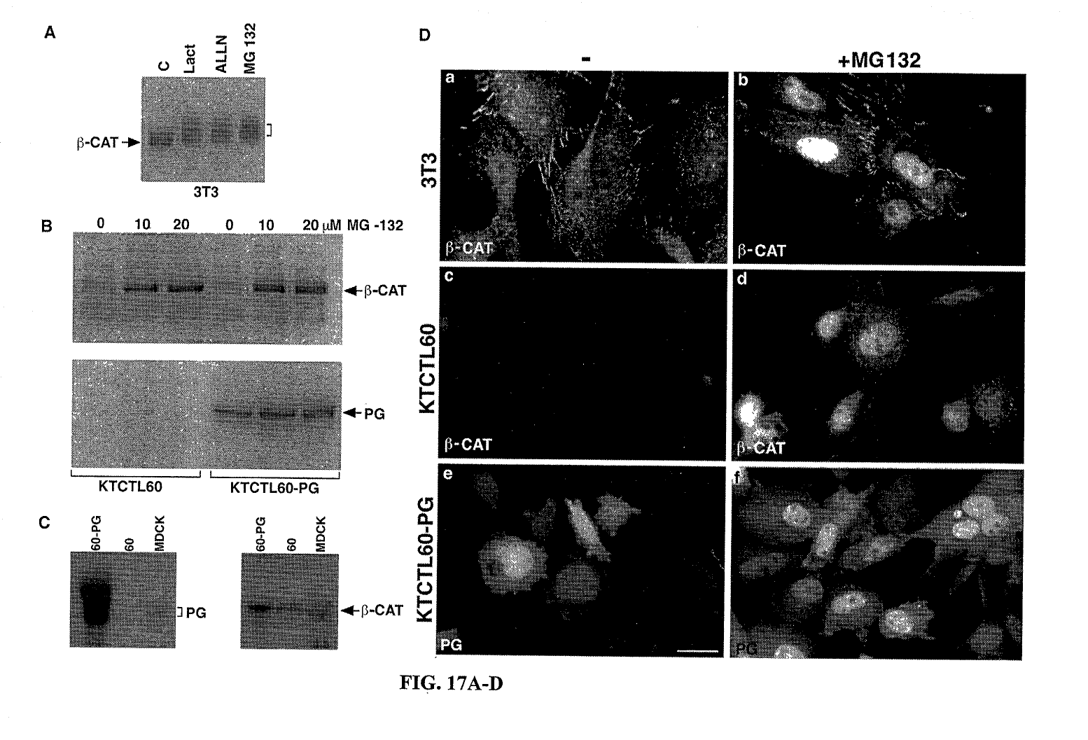

Induction of β-Catenin Accumulation and its Nuclear Localization by Inhibition of the Ubiquitin-Proteasome System: Another treatment by which β-catenin and plakoglobin content could be elevated in cells, is the inhibition of degradation by the ubiquitin-proteasome pathway that apparently controls β-catenin and plakoglobin turnover. Two cell lines were are herein: 3T3 cells that express β-catenin, and KTCTL60 renal carcinoma cells that do not express detectable levels of cadherin, α-, β-catenin, or plakoglobin, and treated them with various inhibitors of the ubiquitin-proteasome system (FIGS. 17A-D). In 3T3 cells, such treatment resulted in the appearance of higher molecular weight β-catenin forms representing most likely ubiquitinated derivatives of the molecule (FIG. 17A). This was accompanied by the accumulation of β-catenin in the nuclei of the cells (FIG. 17D, inset b, compare to inset a). In KTCTL60 cells that contain minute levels of β-catenin, inhibitors of the ubiquitin-proteasome pathway induced a dramatic increase in the level of β-catenin (FIG. 17B), and its translocation to the nucleus (FIG. 17D, inset d, compare to inset c). As these cells express no cadherins, it is conceivable that free β-catenin is very unstable and rapidly degraded by the proteasome pathway in these cells. In contrast, no plakoglobin was detected in KTCTL60 cells either before, or after treating the cells with the proteasome inhibitors (FIG. 17B) due to lack of plakoglobin RNA in these cells (FIG. 17C). When plakoglobin was stably overexpressed in KTCTL60-PG cells (FIG. 17C), its level was further increased by inhibitors of the ubiquitin-proteasome system (FIG. 17B), and it accumulated in the nuclei of the cells (FIG. 17D, insets e and f). These results demonstrate that in some cells the level of β-catenin can be dramatically enhanced by inhibiting its degradation by the ubiquitin-proteasome pathway. Plakoglobin levels were also enhanced by MG-132 treatment, but to a considerably lower extent. Under conditions of excess, both proteins accumulated in the nuclei of cells.

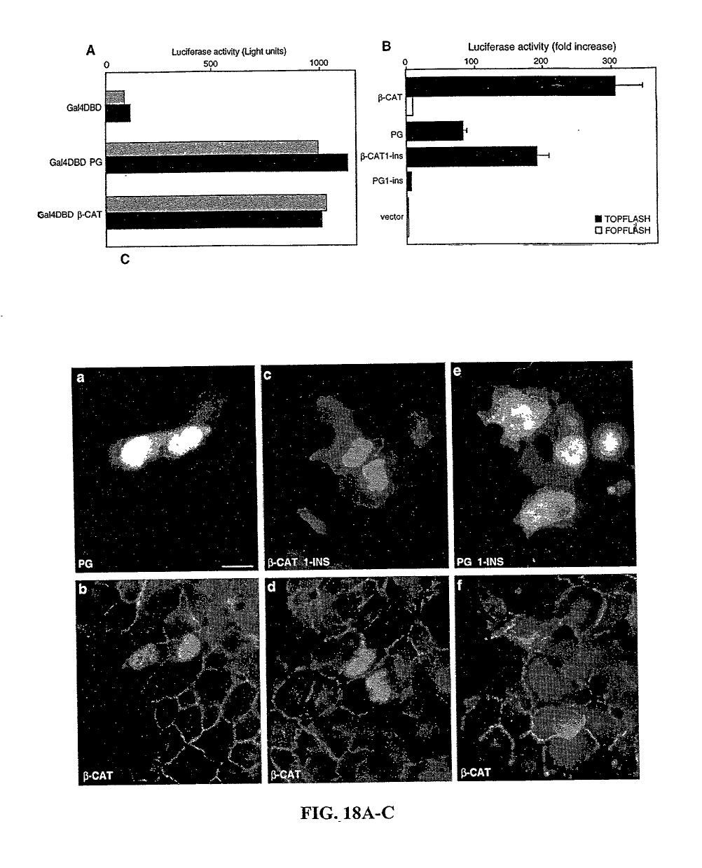

Transcriptional Co-activation by Plakoglobin and β-Catenin of Gal4- and LEF-1-driven Transcription: β-Catenin and its homolog in Drosophila, armadillo, were shown to be able to activate transcription of LEF/TCF-responsive consensus sequences by their COOH-terminus. To compare the ability of β-catenin to that of plakoglobin in transactivation, the COOH-terminus of β-catenin and the corresponding domain in plakoglobin were fused to the Gal4 DNA-binding domain (FIG. 9). Both constructs, when co-transfected with a reporter gene (luciferase) whose transcription was driven by a Gal4-responsive element, showed a similar ability to activate the expression of the reporter gene (FIG. 18A). This implies that the COOH-terminal domain of plakoglobin, like that of armadillo and β-catenin, has the ability to activate transcription.

Next, the capacity of β-catenin and plakoglobin to activate transcription of a reporter gene driven by a multimeric LEF-1 binding consensus sequence in 293 cells was compared. The results summarized in FIG. 18B demonstrate that β-catenin is a potent transcriptional co-activator of the multimeric LEF-1-responsive sequence. Interestingly, a mutant β-catenin lacking the COOH-transactivation domain (FIG. 9, HA β-catenin 1-ins) was also active in promoting LEF-1-driven transcription (FIG. 18B). It was examined whether this resulted from the substitution for endogenous β-catenin in its complexes with cadherin and APC by the mutant β-catenin as seen in Xenopus embryos injected with mutant β-catenin. This could release endogenous β-catenin from cytoplasmic complexes, resulting in its translocation into the nucleus and transcriptional activation of the LEF-1-responsive reporter. Double immunofluorescence using an antibody against the HA-tag linked to the mutant β-catenin (FIG. 18C, inset c) and an anti β-catenin antibody recognizing the COOH-terminus of endogenous β-catenin (but not the mutant HA β-catenin 1-ins which lacks this domain), demonstrated that the level of endogenous β-catenin was elevated, and part of the endogenous protein translocated into the nucleus in cells expressing mutant β-catenin (FIG. 18C, inset d).

Plakoglobin could also activate LEF-1-driven transcription, albeit at 3 to 4 fold lower extent than β-catenin (FIG. 18B). A mutant plakoglobin lacking the COOH-transactivation domain (FIG. 9, HA plakoglobin 1-ins), was unable to enhance LEF-1-driven transcription (FIG. 18B). To examine if full length or mutant plakoglobin overexpression resulted in nuclear accumulation of endogenous β-catenin, cells transfected with HA-tagged plakoglobin were doubly stained for HA (FIG. 18C, inset a), and β-catenin (FIG. 18C, inset b). The results demonstrated that plakoglobin overexpression resulted in nuclear translocation of endogenous β-catenin (FIG. 18C, inset b compare to inset a). In contrast, the COOH-deletion mutant plakoglobin that was abundantly expressed in the transfected cells (FIG. 18C, inset e), was unable to cause translocation of endogenous β-catenin into the nucleus (FIG. 18C, inset f).

Taken together, these results strongly suggest that while both plakoglobin and β-catenin have a COOH-terminal domain that can act as co-transcriptional activator when fused to the Gal4 DNA-binding domain, LEF-1-driven transcriptional activation by mutant β-catenin and wt plakoglobin mostly resulted from the release of endogenous β-catenin from its cytoplasmic partners, its nuclear translocation, and induction of LEF-1-responsive transcription. Thus, elevated plakoglobin expression can influence β-catenin-driven transactivation.

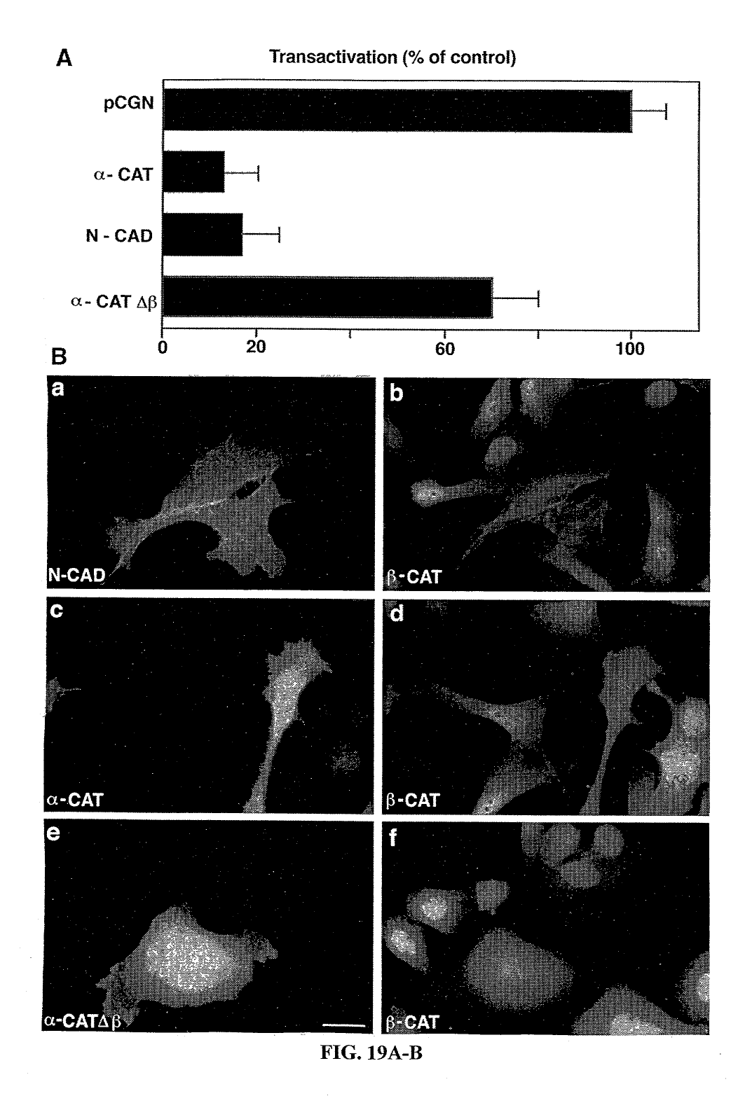

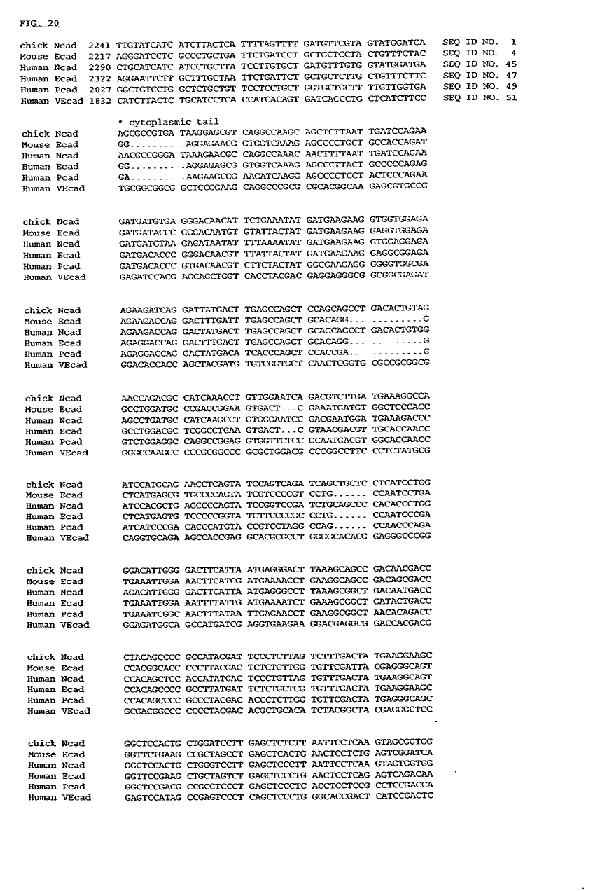

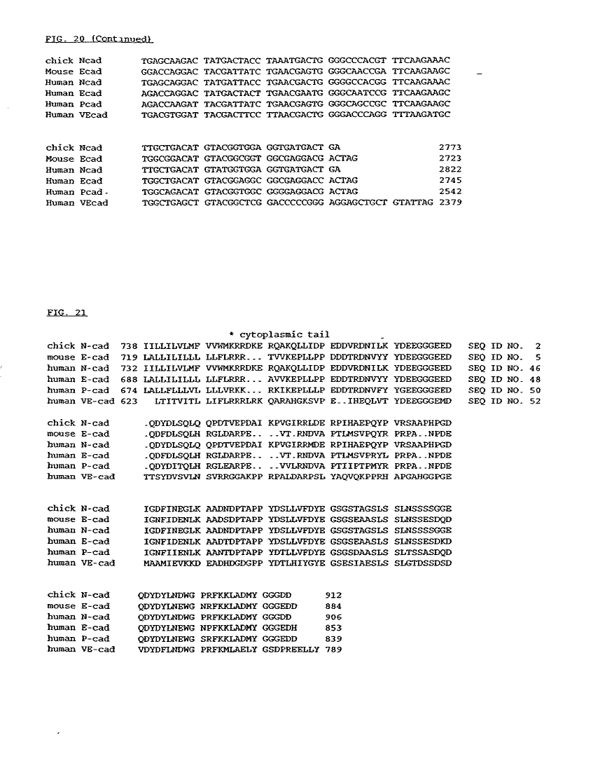

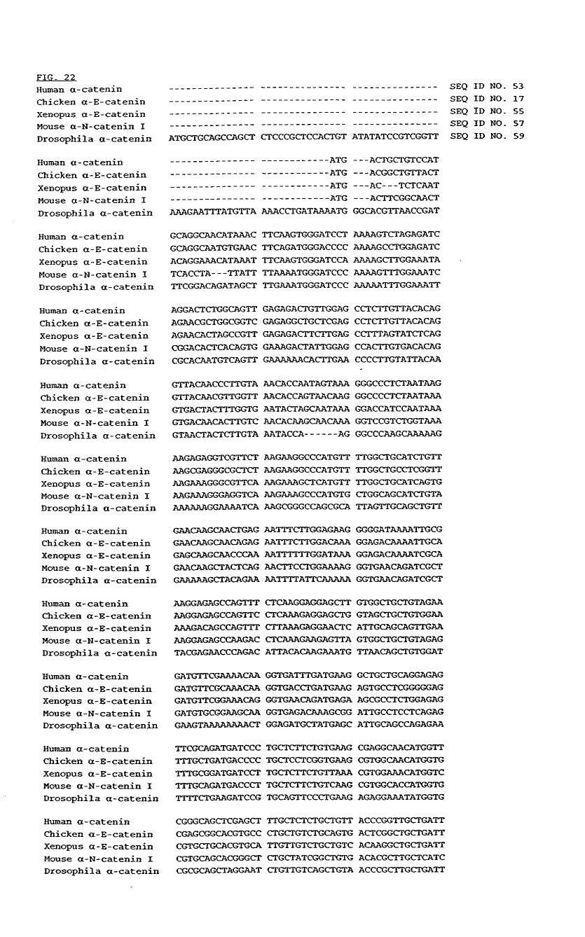

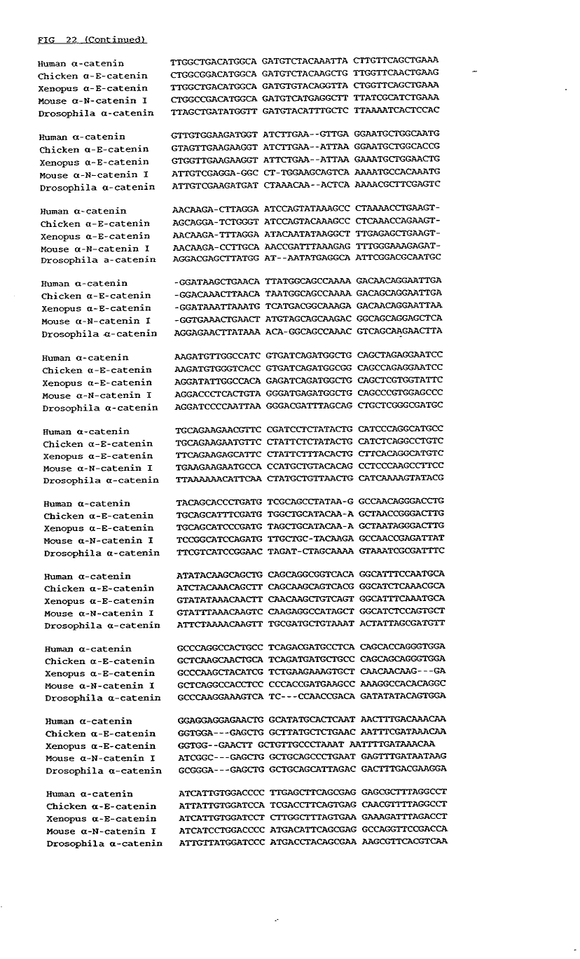

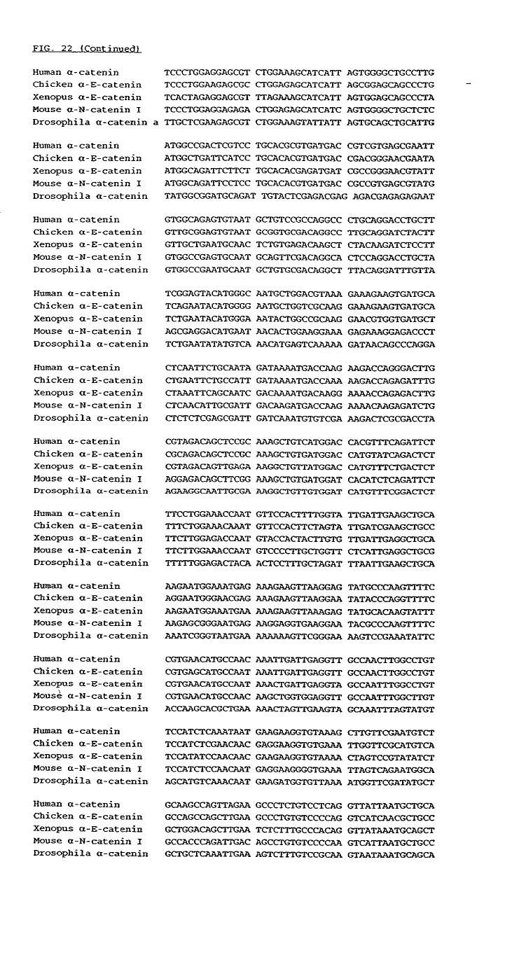

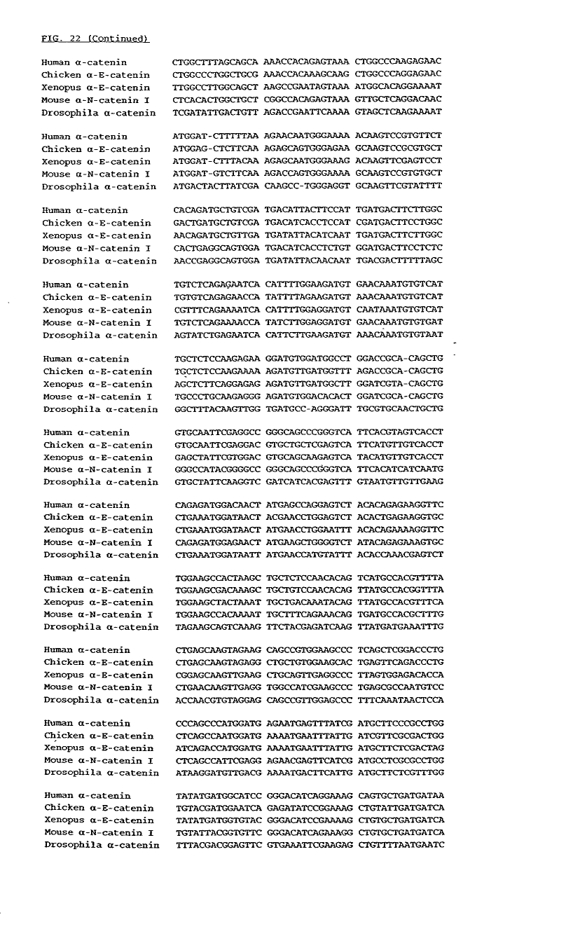

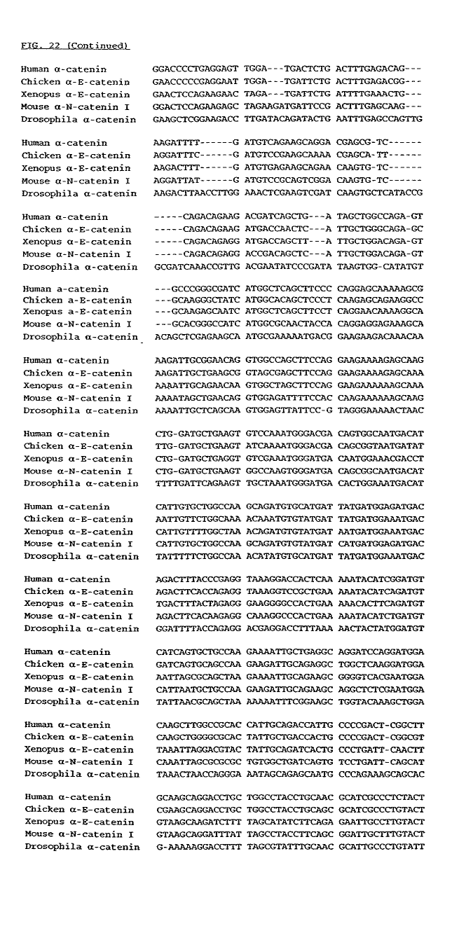

Inhibition Of β-Catenin Nuclear Localization and Transactivation Capacity by N-Cadherin and α-Catenin: Constitutive transactivation by high levels of β-catenin was suggested to be involved in tumor progression in colon carcinoma. In addition, the signaling activity of β-catenin in Xenopus development could be blocked by its junctional partners (i.e., C-cadherin and the NH₂-terminal of α-catenin. The localization and transcriptional activation capacity of β-catenin was investigated in SW480 colon carcinoma cells that overexpress β-catenin due to lack of APC, before and after transfection with N-cadherin and α-catenin. In these cells, β-catenin is abundant in the nucleus and a high level of constitutive LEF-1 driven transcription was detected (FIG. 19A). This activity of β-catenin was effectively blocked by the co-transfection of N-cadherin, or α-catenin (FIG. 19A). Deletion of the β-catenin binding site on α-catenin (FIG. 9, α-catenin Δβ) abolished the transactivation inhibition capacity of this molecule (FIG. 19A). Double immunofluorescence microscopy indicated that both molecules can drive β-catenin out of the nucleus in transfected SW480 cells (FIG. 19B). In these cells, β-catenin was sequestered to the cytoplasm by α-catenin (FIG. 19B, insets c and d) or to cell-cell junctions by the transfected N-cadherin (FIG. 19B, insets a and b). The α-catenin mutant lacking the β-catenin binding site (FIG. 19B, insets e and f) did not show this activity. The results suggest that the partners of β-catenin that are active in cell adhesion, are effective antagonists of the nuclear localization of β-catenin and its function in transcriptional regulation.

### EXAMPLE 3

### Sequence Homologies among Cadherin and o-Catenin Genes and Proteins

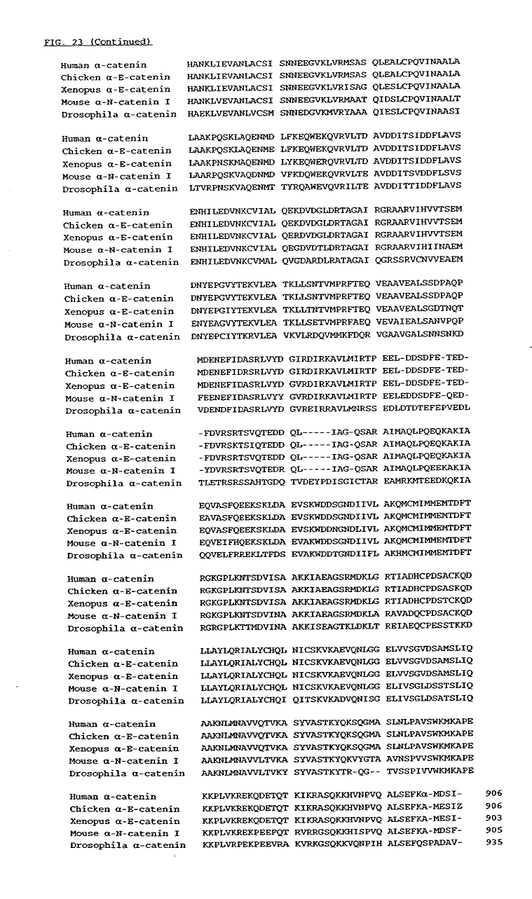

It is evident from the experiment described herein and elsewhere that both cadherins and o catenins are functionally conserved. This conservation specifically includes their ability for interspecies interaction with β catenins, as was determined using various heterologous systems. As can be expected, this functional conservation is reflected by high sequence conservation at the nucleic and amino acid levels among cadherins and o catenins of different types and origins, as is evident from the homologies presented in FIGS. 20-23.

Thus, the scope of the present invention is not limited to cadherins and o catenins of specific types and origins, rather, it is intended to embrace all cadherins and o catenins regardless of their type and species origin because all of the cadherins and o catenins so far examined include interspecies functional β catenin binding domains.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A pharmaceutical composition for treatment of cancer associated with abnormally high activity levels of β-catenin comprising a pharmaceutically acceptable gene therapy vehicle harboring a polynucleotide that comprises:
(a) a first nucleotide sequence encoding a soluble cytoplasmic portion of a cadherin, said soluble cytoplasmic portion of said cadherin lacking a transmembrane portion and an extracellular portion of said cadherin and including a β-catenin binding domain; and
(b) a second nucleotide sequence being positioned upstream of said first nucleotide sequence and containing a promoter for directing expression of said soluble cytoplasmic portion of said cadherin in a mammalian cell;
said acceptable gene therapy vehicle being therapeutically effective in reducing the abnormally high activity levels of β-catenin.

2. The pharmaceutical composition of claim 1, wherein said cadherin is selected from the group consisting of E-cadherin, N-cadherin, P-cadherin and VE-cadherin.

3. The pharmaceutical composition of claim 1, wherein said first nucleotide sequence is derived from SEQ ID NOs. 1, 4, 45, 47, 49 or 51.

4. The pharmaceutical composition of claim 1, wherein said first nucleotide sequence encodes, at most, about 70 amino acids of a cadherin.

5. The pharmaceutical composition of claim 1, wherein said cadherin is from a species selected from the group consisting of human, chicken, xenopus, mouse, canine and drosophila.

6. The pharmaceutical composition of claim 1, wherein said cadherin is human.

7. The pharmaceutical composition of claim 10, wherein said cytoplasmic portion of said cadherin is signal peptide-free.

8. A pharmaceutical composition for treatment of cancer associated with abnormally high activity levels of β-catenin comprising a pharmaceutically acceptable gene therapy vehicle harboring a polynucleotide that comprises:
(a) a first nucleotide sequence encoding an o-catenin; and
(b) a second nucleotide sequence being positioned upstream of said first nucleotide sequence and containing a promoter for directing expression of said o-catenin in a mammalian cell;
said acceptable gene therapy vehicle being therapeutically effective in reducing the abnormally high activity levels of β-catenin.

9. The pharmaceutical composition of claim 8, wherein said o-catenin is from human.

10. A method of treating cancer associated with abnormally high activity levels of β-catenin comprising administering to a subject in need a pharmaceutically acceptable gene therapy vehicle harboring a polynucleotide that comprises:
(a) a first nucleotide sequence encoding a soluble cytoplasmic portion of a cadherin, said soluble cytoplasmic portion of said cadherin lacking a transmembrane portion and an extracellular portion of said cadherin and including a β-catenin binding domain; and
(b) a second nucleotide sequence being positioned upstream of said first nucleotide sequence and containing a promoter for directing expression of said soluble cytoplasmic portion of said cadherin in a mammalian cell;
said acceptable gene therapy vehicle being therapeutically effective in reducing the abnormally high activity levels of β-catenin.

11. The method of claim 10, wherein said cadherin is selected from the group consisting of E-cadherin, N-cadherin, P-cadherin and VE-cadherin.

12. The method of claim 10, wherein said first nucleotide sequence is derived from SEQ ID NOs. 1, 4, 45, 47, 49 or 51.

13. The method of claim 10, wherein said first nucleotide sequence encodes, at most, about 70 amino acids of a cadherin.

14. The method of claim 10, wherein said cadherin is from a species selected from the group consisting of human, chicken, xenopus, mouse, canine and drosophila.

15. The method of claim 10, wherein said cadherin is human.

16. The method of claim 10, wherein said cytoplasmic portion of said cadherin is signal peptide-free.

17. A method of treating cancer associated with abnormally high activity levels of β-catenin comprising administering to a subject in need a pharmaceutically acceptable gene therapy vehicle harboring a polynucleotide that comprises:
(a) a first nucleotide sequence encoding an o-catenin; and
(b) a second nucleotide sequence being positioned upstream of said first nucleotide sequence and containing a promoter for directing expression of said c-catenin in a mammalian cell;
said acceptable gene therapy vehicle being therapeutically effective in reducing the abnormally high activity levels of β-catenin.

18. The method of claim 17, wherein said o-catenin is from human.

19. A method reducing abnormally high activity levels of β-catenin is mammalian cells comprising infecting or transforming the cells with a vehicle harboring a polynucleotide that comprises:
(a) a first nucleotide sequence encoding a soluble cytoplasmic portion of a cadherin, said soluble cytoplasmic portion of said cadherin lacking a transmembrane portion and an extracellular portion of said cadherin and including a β-catenin binding domain; and
(b) a second nucleotide sequence being positioned upstream of said first nucleotide sequence and containing a promoter for directing expression of said soluble cytoplasmic portion of said cadherin in a mammalian cell;
said vehicle being effective in reducing the abnormally high activity levels of β-catenin in the cells.

20. The method of claim 19, wherein said cadherin is selected from the group consisting of E-cadherin, N-cadherin, P-cadherin and VE-cadherin.

21. The method of claim 19, wherein said first nucleotide sequence is derived from SEQ ID NOs. 1, 4, 45, 47, 49 or 51.

22. The method of claim 19, wherein said first nucleotide sequence encodes, at most, about 70 amino acids of a cadherin.

23. The method of claim 19, wherein said cadherin is from a species selected from the group consisting of human, chicken, xenopus, mouse, canine and drosophila.

24. The method of claim 19, wherein said cadherin is human.

25. The method of claim 19, wherein said cytoplasmic portion of said cadherin is signal peptide-free.

26. A method reducing abnormally high activity levels of β-catenin is mammalian cells comprising infecting or transforming the cells with a vehicle harboring a polynucleotide that comprises:
(a) a first nucleotide sequence encoding an o-catenin; and
(b) a second nucleotide sequence being positioned upstream of said first nucleotide sequence and containing a promoter for directing expression of said o-catenin in a mammalian cell;
said vehicle being effective in reducing the abnormally high activity levels of β-catenin in the cells.

27. The method of claim 26, wherein said o-catenin is from human.


SEQUENCE LISTING (60 sequences)

SEQ ID NO: 1 - DNA, 2824 bp, Gallus gallus
gggggccgccccgccgccgcccctcctcgcctccatgtgccggatagcgggaacgccgccgcggatcctgccgccgctggcgctgatgctgctggcggccctgcagcaggcaccgataaaagcaacttgtgaagacatgttgtgcaagatgggatttcctgaagatgtgcacagtgcagtcgtgtcgaggagtgtacatggaggacaacctctgctcaatgtgaggtttcaaagctgcgatgaaaacagaaaaatatactttggaagcagtgagccagaagattttagagtaggtgaagatggtgtggtatatgcagagagaagctttcaactttcagcagagcccacggagtttgtagtgtctgctcgagacaaggaaactcaggaagaatggcaaatgaaggtgaagctaacccctgaaccagcattcacaggggcctcagaaaaggaccaaaagaaaattgaagacatcatatttccatggcaacaatataaggacagcagccatctgaagagacagaagagagactgggttatccctccaatcaacctaccagaaaattccagaggaccttttcctcaagaattagttaggattcggtctgatcgtgataaaagcctttcgctacggtacagtgtgactggcccaggagctgaccaacctccaacaggaatcttcatcatcaaccccatctcaggacagctgtctgtgacaaagcctttagatcgggagcagattgcttcttttcatctgagagcacatgcagtggatgtaaatggaaaccaggtggaaaatcctattgatattgtgattaacgtcattgatatgaatgataacagacctgaattcttgcatcaggtttggaatgggacagttcctgaaggatcaaagccaggaacctatgtaatgactgttactgccatcgatgctgatgatcccaatgcacagaatgggatgctgagatacagaatcttgtcacaggcaccaagcagtccctctcccaacatgtttacaatcaacaatgagactggtgacattattaccgtagcagctgggctcgacagagagaaagtacaacagtatacattaataattcaagctacggacatggaaggaaacccaacatatggtctttcaaacacagcaactgctgtcatcactgtgacagatgtcaatgacaatcctccagagttcactgctatgactttctacggtgaagtaccagaaaacagagtggatgtcatagtggctaacctaacagtaacagataaagatcagccacacacgcctgcgtggaatgcgaggtaccaaatgacagggggagacccgacaggccagtttactatcctgaccgatccaaatagcaatgatgggttggtaactgttgtcaagcccattgactttgagaccaacaggatgtttgtacttactgtagctgcagaaaatcaagtgcctttggctaaggggattcagcatcctcctcagtcaacagcaaccgtgtccattacagtcattgatgtgaatgagagtccatattttgttccaaaccccaagcttgtacgtcaagaagaagggctacttgctggtagcatgttgacaactttcactgctcgggacccagatcgttacatgcagcaaacctctctaaggtactcaaaactttcggaccctgcaaactggctaaaaattgaccctgttaatggacaaataacaaccacagctgttttggacagagaatcgatatatgtgcaaaacaatatgtataatgcaacttttcttgcctctgataatggaattcctccaatgagtggaactggtacacttcagatatacttgctggacatcaatgataatgctccccaagtgaacccaaaagaagccaccacctgtgaaacactgcagcctaatgctattaacatcactgctgtagaccctgacattgatccaaatgcaggcccatttgcctttgagctgcctgattcacctcctagtattaagaggaattggaccattgttcgaattagtggtgatcatgcccagctctctttaaggatcaggttcctggaggctggtatctatgatgtgcccatagtaattacagattctggaaatccacatgcatctagcacttctgtgctaaaagtgaaagtttgccaatgtgacataaatggggactgtactgatgttgaccggattgttggcgcaggactgggcactggtgccatcattgcaattctgctttgtatcatcatcttactcattttagttttgatgttcgtagtatggatgaagcgccgtgataaggagcgtcaggccaagcagctcttaattgatccagaagatgatgtgagggacaacattctgaaatatgatgaagaaggtggtggagaagaagatcaggattatgacttgagccagctccagcagcctgacactgtagaaccagacgccatcaaacctgttggaatcagacgtcttgatgaaaggccaatccatgcagaacctcagtatccagtcagatcagctgctcctcatcctggggacattggggacttcattaatgagggacttaaagcagccgacaacgaccctacagccccgccatacgattccctcttagtctttgactatgaaggaagcggctccactgctggatccttgagctctcttaattcctcaagtagcggtggtgagcaagactatgactacctaaatgactggggcccacgtttcaagaaacttgctgacatgtacggtggaggtgatgactgaacttcaaagtgaactttgtttctggacaagtacaaacatttcaactgatat

SEQ ID NO: 2 - protein, 912 aa, Gallus gallus
MCRIAGTPPRILPPLALMLLAALQQAPIKATCEDMLCKMGFPEDVHSAVVSRSVHGGQPLLNVRFQSCDENRKIYFGSSEPEDFRVGEDGVVYAERSFQLSAEPTEFVVSARDKETQEEWQMKVKLTPEPAFTGASEKDQKKIEDIIFPWQQYKDSSHLKRQKRDWVIPPINLPENSRGPFPQELVRIRSDRDKSLSLRYSVTGPGADQPPTGIFIINPISGQLSVTKPLDREQIASFHLRAHAVDVNGNQVENPIDIVINVIDMNDNRPEFLHQVWNGTVPEGSKPGTYVMTVTAIDADDPNAQNGMLRYRILSQAPSSPSPNMFTINNETGDIITVAAGLDREKVQQYTLIIQATDMEGNPTYGLSNTATAVITVTDVNDNPPEFTAMTFYGEVPENRVDVIVANLTVTDKDQPHTPAWNARYQMTGGDPTGQFTILTDPNSNDGLVTVVKPIDFETNRMFVLTVAAENQVPLAKGIQHPPQSTATVSITVIDVNESPYFVPNPKLVRQEEGLLAGSMLTTFTARDPDRYMQQTSLRYSKLSDPANWLKIDPVNGQITTTAVLDRESIYVQNNMYNATFLASDNGIPPMSGTGTLQIYLLDINDNAPQVNPKEATTCETLQPNAINITAVDPDIDPNAGPFAFELPDSPPSIKRNWTIVRISGDHAQLSLRIRFLEAGIYDVPIVITDSGNPHASSTSVLKVKVCQCDINGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRLDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 3 - DNA, 2824 bp, Gallus gallus; identical to SEQ ID NO: 1
  CDS (35)..(2770)

SEQ ID NO: 4 - DNA, 2768 bp, Mus musculus
ccggcctaacccggccctgcccgaccgcacccgagctcagtgtttgctcggcgtctgccgggtccgccatgggagcccggtgccgcagcttttccgcgctcctgctcctgctgcaggtctcctcatggctttgccaggagctggagcctgagtcctgcagtcccggcttcagttccgaggtctacaccttcccggtgccggagaggcacctggagagaggccatgtcctgggcagagtgagatttgaaggatgcaccggccggccaaggacagccttcttttcggaagactcccgattcaaagtggcgacagacggcaccatcacagtgaagcggcatctaaagctccacaagctggagaccagtttcctcgtccgcgcccgggactccagtcatagggagctgtctaccaaagtgacgctgaagtccatggggcaccaccatcaccggcaccaccaccgcgaccctgcctctgaatccaacccagagctgctcatgtttcccagcgtgtacccaggtctcagaagacagaaacgagactgggtcatccctcccatcagctgccccgaaaatgaaaagggtgaattcccaaagaacctggttcagatcaaatccaacagggacaaagaaacaaaggttttctacagcatcaccggccaaggagctgacaaaccccccgttggcgttttcatcattgagagggagacaggctggctgaaagtgacacagcctctggatagagaagccattgccaagtacatcctctattctcatgccgtgtcatcaaatggggaagcggtggaggatcccatggagatagtgatcacagtgacagatcagaatgacaacaggccagagtttacccaggaggtgtttgagggatccgttgcagaaggcgctgttccaggaacctccgtgatgaaggtctcagccaccgatgcagacgatgacgtcaacacctacaacgctgccatcgcctacaccatcgtcagccaggatcctgagctgcctcacaaaaacatgttcactgtcaatagggacaccggggtcatcagtgtgctcacctctgggctggaccgagagagttaccctacatacactctggtggttcaggctgctgaccttcaaggcgaaggcttgagcacaacagccaaggctgtgatcactgtcaaggatattaatgacaacgctcctgtcttcaacccgagcacgtatcagggtcaagtgcctgagaatgaggtcaatgcccggatcgccacactcaaagtgaccgatgatgatgcccccaacactccggcgtggaaagctgtgtacaccgtagtcaacgatcctgaccagcagttcgttgtcgtcacagaccccacgaccaatgatggcattttgaaaacagccaagggcttggattttgaggccaagcagcaatacatccttcatgtgagagtggagaacgaggaaccctttgaggggtctcttgtcccttccacagccactgtcactgtggacgtggtagacgtgaatgaagcccccatctttatgcctgcggagaggagagtcgaagtgcccgaagactttggtgtgggtcaggaaatcacatcttataccgctcgagagccggacacgttcatggatcagaagatcacgtatcggatttggagggacactgccaactggctggagattaacccagagactggtgccattttcacgcgcgctgagatggacagagaagacgctgagcatgtgaagaacagcacatatgtagctctcatcatcgccacagatgatggttcacccattgccactggcacgggcactcttctcctggtcctgttagacgtcaatgacaacgctcccatcccagaacctcgaaacatgcagttctgccagaggaacccacagcctcatatcatcaccatcttggatccagaccttccccccaacacgtccccctttactgctgagctaacccatggggccagcgtcaactggaccattgagtataatgacgcagctcaagaatctctcattttgcaaccaagaaaggacttagagattggcgaatacaaaatccatctcaagctcgcggataaccagaacaaagaccaggtgaccacgttggacgtccatgtgtgtgactgtgaagggacggtcaacaactgcatgaaggcgggaatcgtggcagcaggattgcaagttcctgccatcctcggaatccttggagggatcctcgccctgctgattctgatcctgctgctcctactgtttctacggaggagaacggtggtcaaagagcccctgctgccaccagatgatgatacccgggacaatgtgtattactatgatgaagaaggaggtggagaagaagaccaggactttgatttgagccagctgcacaggggcctggatgcccgaccggaagtgactcgaaatgatgtggctcccaccctcatgagcgtgccccagtatcgtccccgtcctgccaatcctgatgaaattggaaacttcatcgatgaaaacctgaaggcagccgacagcgaccccacggcacccccttacgactctctgttggtgttcgattacgagggcagtggttctgaagccgctagcctgagctcactgaactcctctgagtcggatcaggaccaggactacgattatctgaacgagtggggcaaccgattcaagaagctggcggacatgtacggcggtggcgaggacgactaggggactagcaagtctcccccgtgtggcaccatgggagatgcagaa

SEQ ID NO: 5 - protein, 899 aa, Mus musculus
TAPELSVCSASAGSAMGARCRSFSALLLLLQVSSWLCQELEPESCSPGFSSEVYTFPVPERHLERGHVLGRVRFEGCTGRPRTAFFSEDSRFKVATDGTITVKRHLKLHKLETSFLVRARDSSHRELSTKVTLKSMGHHHHRHHHRDPASESNPELLMFPSVYPGLRRQKRDWVIPPISCPENEKGEFPKNLVQIKSNRDKETKVFYSITGQGADKPPVGVFIIERETGWLKVTQPLDREAIAKYILYSHAVSSNGEAVEDPMEIVITVTDQNDNRPEFTQEVFEGSVAEGAVPGTSVMKVSATDADDDVNTYNAAIAYTIVSQDPELPHKNMFTVNRDTGVISVLTSGLDRESYPTYTLVVQAADLQGEGLSTTAKAVITVKDINDNAPVFNPSTYQGQVPENEVNARIATLKVTDDDAPNTPAWKAVYTVVNDPDQQFVVVTDPTTNDGILKTAKGLDFEAKQQYILHVRVENEEPFEGSLVPSTATVTVDVVDVNEAPIFMPAERRVEVPEDFGVGQEITSYTAREPDTFMDQKITYRIWRDTANWLEINPETGAIFTRAEMDREDAEHVKNSTYVALIIATDDGSPIATGTGTLLLVLLDVNDNAPIPEPRNMQFCQRNPQPHIITILDPDLPPNTSPFTAELTHGASVNWTIEYNDAAQESLILQPRKDLEIGEYKIHLKLADNQNKDQVTTLDVHVCDCEGTVNNCMKAGIVAAGLQVPAILGILGGILALLILILLLLLFLRRRTVVKEPLLPPDDDTRDNVYYYDEEGGGEEDQDFDLSQLHRGLDARPEVTRNDVAPTLMSVPQYRPRPANPDEIGNFIDENLKAADSDPTAPPYDSLLVFDYEGSGSEAASLSSLNSSESDQDQDYDYLNEWGNRFKKLADMYGGGEDD

SEQ ID NO: 6 - DNA, 2768 bp, Mus musculus; identical to SEQ ID NO: 4
  CDS (24)..(2720)

SEQ ID NO: 7 - DNA, 29 bp, Artificial sequence
  Synthetic oligonucleotide
cggaattccaagcgccgtgataaggagcg

SEQ ID NO: 8 - DNA, 31 bp, Artificial sequence
  Synthetic oligonucleotide
gctctagatcagtcatagtcttgctcaccac

SEQ ID NO: 9 - DNA, 29 bp, Artificial sequence
  Synthetic oligonucleotide
cggaattccattaatgagggacttaaagc

SEQ ID NO: 10 - DNA, 29 bp, Artificial sequence
  Synthetic oligonucleotide
cggaattccttagtctttgactatgaagg

SEQ ID NO: 11 - DNA, 31 bp, Artificial sequence; identical to SEQ ID NO: 8
  Synthetic oligonucleotide

SEQ ID NO: 12 - DNA, 29 bp, Artificial sequence
  Synthetic oligonucleotide
cggaattccaggagaacggtggtcaaaga

SEQ ID NO: 13 - DNA, 28 bp, Artificial sequence
  Synthetic oligonucleotide
gctctagactagtcgtcctcgccaccgc

SEQ ID NO: 14 - DNA, 29 bp, Artificial sequence
  Synthetic oligonucleotide
cggaattccatcgatgaaaacctgaaggc

SEQ ID NO: 15 - DNA, 29 bp, Artificial sequence
  Synthetic oligonucleotide
cggaattccttggtgttcgattacgaggg

SEQ ID NO: 16 - DNA, 31 bp, Artificial sequence
  Synthetic oligonucleotide
gctctagatcaatcgtagtcctggtcctgat

SEQ ID NO: 17 - DNA, 2718 bp, Gallus gallus
atgacggctgttactgcaggcaatgtgaacttcagatgggaccccaaaagcctggagatcagaacgctggcggtcgagaggctgctcgagcctcttgttacacaggttacaacgttggttaacaccagtaacaagggcccctctaataaaaagcgagggcgctctaagaaggcccatgttttggctgcctcggttgaacaagcaacagagaatttcttggacaaaggagacaaaattgcaaaggagagccagttcctcaaagaggagctggtagctgctgtggaagatgttcgcaaacaaggtgacctgatgaagagtgcctcgggggagtttgctgatgacccctgctcctcggtgaagcgtggcaacatggtgcgagcggcacgtgccctgctgtctgcagtgactcggctgctgattctggcggacatggcagatgtctacaagctgttggttcaactgaaggtagttgaagaaggtatcttgaaattaaggaatgctggcaccgagcaggatctgggtatccagtacaaagccctcaaaccagaagtggacaaacttaacataatggcagccaaaagacagcaggaattgaaagatgtgggtcaccgtgatcagatggcggcagccagaggaatcctgcagaagaatgttcctattctctatactgcatctcaggcctgtctgcagcatttcgatgtggctgcatacaaagctaaccgggacttgatctacaaacagcttcagcaagcagtcacgggcatctcaaacgcagctcaagcaactgcatcagatgatgctgcccagcagcagggtggaggtggagagctggcttatgctctgaacaatttcgataaacaaattattgtggatccatcgaccttcagtgagcaacgttttaggccttccctggaagagcgcctggagagcatcattagcggagcagccctgatggctgattcatcctgcacacgtgatgaccgacgggaacgaatagttgcggagtgtaatgcggtgcgacaggccttgcaggatctactttcagaatacatggggaatgctggtcgcaaggaaagaagtgatgcactgaattctgccattgataaaatgaccaaaaagaccagagatttgcgcagacagctccgcaaagctgtgatggaccatgtatcagactcttttctggaaacaaatgttccacttctagtattgatcgaagctgccaggaatgggaacgagaaagaagttaaggaatatacccaggttttccgtgagcatgccaataaattgattgaggttgccaacttggcctgttccatctcgaacaacgaggaaggtgtgaaattggttcgcatgtcagccagccagcttgaagccctgtgtccccaggtcatcaacgctgccctggccctggctgcgaaaccacaaagcaagctggcccaggagaacatggagctcttcaaagagcagtgggagaagcaagtccgcgtgctgactgatgctgtcgatgacatcacctccatcgatgacttcctggctgtgtcagagaaccatattttagaagatgtaaacaaatgtgtcattgctctccaagaaaaagatgttgatggtttagaccgcacagctggtgcaattcgaggacgtgctgctcgagtcattcatgttgtcacctctgaaatggataactacgaacctggagtctacactgagaaggtgctggaagcgacaaagctgctgtccaacacagttatgccacggtttactgagcaagtagaggctgctgtggaagcactgagttcagaccctgctcagccaatggatgaaaatgaatttattgatcgttcgcgactggtgtacgatggaatcagagatatccggaaagctgtattgatgatcagaacccccgaggaattggatgattctgactttgagacggaggatttcgatgtccgaagcaaaacgagcattcagacagaagatgaccaactcattgctgggcagagcgcaagggctatcatggcacagctccctcaagagcagaaggccaagattgctgaagcggtagcgagcttccaggaagaaaagagcaaattggatgctgaagtatcaaaatgggacgacagcggtaatgatataattgttctggcaaaacaaatgtgtatgattatgatggaaatgacagacttcaccagaggtaaaggtccgctgaaaaatacatcagatgtgatcagtgcagccaagaagattgcagaggctggctcaaggatggacaagctggggcgcactattgctgaccactgccccgactcggcgtcgaagcaggacctgctggcctacctgcagcgcatcgccctgtactgccaccagctcaacatctgcagcaaagtgaaggccgaagtgcagaacctcggaggggagctcgtcgtgtctggggtggacagcgccatgtccctcatccaggcggccaagaacctgatgaacgccgtggtgcagacggtgaaggcgtcctacgtggcgtccaccaagtaccagaagtcgcagggcatggcctcgctcaacctccccgccgtgtcctggaagatgaaggctccggagaagaagcccctggtcaagagggagaagcaggacgagacccagaccaaaatcaagcgggcgtcccagaagaagcacgtcaacccggtgcaggcgctcagcgagttcaaggcgatggagagcatttag

SEQ ID NO: 18 - protein, 905 aa, Gallus gallus
MTAVTAGNVNFRWDPKSLEIRTLAVERLLEPLVTQVTTLVNTSNKGPSNKKRGRSKKAHVLAASVEQATENFLDKGDKIAKESQFLKEELVAAVEDVRKQGDLMKSASGEFADDPCSSVKRGNMVRAARALLSAVTRLLILADMADVYKLLVQLKVVEEGILKLRNAGTEQDLGIQYKALKPEVDKLNIMAAKRQQELKDVGHRDQMAAARGILQKNVPILYTASQACLQHFDVAAYKANRDLIYKQLQQAVTGISNAAQATASDDAAQQQGGGGELAYALNNFDKQIIVDPSTFSEQRFRPSLEERLESIISGAALMADSSCTRDDRRERIVAECNAVRQALQDLLSEYMGNAGRKERSDALNSAIDKMTKKTRDLRRQLRKAVMDHVSDSFLETNVPLLVLIEAARNGNEKEVKEYTQVFREHANKLIEVANLACSISNNEEGVKLVRMSASQLEALCPQVINAALALAAKPQSKLAQENMELFKEQWEKQVRVLTDAVDDITSIDDFLAVSENHILEDVNKCVIALQEKDVDGLDRTAGAIRGRAARVIHVVTSEMDNYEPGVYTEKVLEATKLLSNTVMPRFTEQVEAAVEALSSDPAQPMDENEFIDRSRLVYDGIRDIRKAVLMIRTPEELDDSDFETEDFDVRSKTSIQTEDDQLIAGQSARAIMAQLPQEQKAKIAEAVASFQEEKSKLDAEVSKWDDSGNDIIVLAKQMCMIMMEMTDFTRGKGPLKNTSDVISAAKKIAEAGSRMDKLGRTIADHCPDSASKQDLLAYLQRIALYCHQLNICSKVKAEVQNLGGELVVSGVDSAMSLIQAAKNLMNAVVQTVKASYVASTKYQKSQGMASLNLPAVSWKMKAPEKKPLVKREKQDETQTKIKRASQKKHVNPVQALSEFKAMESI

SEQ ID NO: 19 - DNA, 2718 bp, Gallus gallus; identical to SEQ ID NO: 17
  CDS (1)..(2715)

SEQ ID NO: 20 - DNA, 31 bp, Artificial sequence
  Synthetic oligonucleotide
accttctagaatggctactcaagctgacctg

SEQ ID NO: 21 - DNA, 20 bp, Artificial sequence
  Synthetic oligonucleotide
atgagcagcgtcaaactgcg

SEQ ID NO: 22 - DNA, 34 bp, Artificial sequence
  Synthetic oligonucleotide
accttctagattgaaacatgcagttgtcaatttg

SEQ ID NO: 23 - DNA, 29 bp, Artificial sequence
  Synthetic oligonucleotide
acctggatccagctccagtacacccttcg

SEQ ID NO: 24 - DNA, 18 bp, Artificial sequence
  Synthetic oligonucleotide
aactgctcctcttactga

SEQ ID NO: 25 - DNA, 28 bp, Artificial sequence
  Synthetic oligonucleotide
tatcccgggtcaagtcagtgtcaaacca

SEQ ID NO: 26 - DNA, 32 bp, Artificial sequence
  Synthetic oligonucleotide
accttctagaatggaggtgatgaacctgatgg

SEQ ID NO: 27 - DNA, 22 bp, Artificial sequence
  Synthetic oligonucleotide
agctgagcatgcggaccagagc

SEQ ID NO: 28 - DNA, 29 bp, Artificial sequence
  Synthetic oligonucleotide
accttctagactcaagtcggccattgtgc

SEQ ID NO: 29 - DNA, 29 bp, Artificial sequence
  Synthetic oligonucleotide
acctggatcctgctccggtgcagccctcc

SEQ ID NO: 30 - DNA, 19 bp, Artificial sequence
  Synthetic oligonucleotide
aggccgcccgggcagcatg

SEQ ID NO: 31 - DNA, 21 bp, Artificial sequence
  Synthetic oligonucleotide
cgcatggagatcttccggctc

SEQ ID NO: 32 - DNA, 30 bp, Artificial sequence
  Synthetic oligonucleotide
accttctagaatgacggctgttactgcagg

SEQ ID NO: 33 - DNA, 20 bp, Artificial sequence
  Synthetic oligonucleotide
gccttcttagagcgccctcg

SEQ ID NO: 34 - DNA, 30 bp, Artificial sequence; identical to SEQ ID NO: 32
  Synthetic oligonucleotide

SEQ ID NO: 35 - DNA, 29 bp, Artificial sequence
  Synthetic oligonucleotide
tgccagcggagcaggggtcatcagcaaac

SEQ ID NO: 36 - DNA, 28 bp, Artificial sequence
  Synthetic oligonucleotide
ctgctccgctggcaccgagcaggatctg

SEQ ID NO: 37 - DNA, 20 bp, Artificial sequence
  Synthetic oligonucleotide
acgttgctcactgaaggtcg

SEQ ID NO: 38 - DNA, 30 bp, Artificial sequence; identical to SEQ ID NO: 32
  Synthetic oligonucleotide

SEQ ID NO: 39 - DNA, 20 bp, Artificial sequence
  Synthetic oligonucleotide
acgttgctcactgaaggtcg

SEQ ID NO: 40 - DNA, 31 bp, Artificial sequence
  Synthetic oligonucleotide
accttctagaatgaagctactgtcttctatc

SEQ ID NO: 41 - DNA, 31 bp, Artificial sequence
  Synthetic oligonucleotide
acctgagctccgatacagtcaactgtctttg

SEQ ID NO: 42 - DNA, 33 bp, Artificial sequence
  Synthetic oligonucleotide
acctggatcctacgatacagtcaactgtctttg

SEQ ID NO: 43 - DNA, 36 bp, Artificial sequence
  Synthetic oligonucleotide
ggaagactctcctccggatccggaagactctcctcc

SEQ ID NO: 44 - DNA, 44 bp, Artificial sequence
  Synthetic oligonucleotide
gatcggaggagagtcttccggatccggaggagagtcttccagct

SEQ ID NO: 45 - DNA, 2853 bp, Homo sapiens
accctggccgctgttggtgctgccgctgcctcctcctcctccgccgccgccgccgccgccgccgcctcctccggctcttcgctcggcccctctccgcctccatgtgccggatagcgggagcgctgcggaccctgctgccgctgctgctggccctgcttcaggcgtctgtagaggcttctggtgaaatcgcattatgcaagactggatttcctgaagatgtttacagtgcagtcttatcgaaggatgtgcatgaaggacagcctcttctcaatgtgaagtttagcaactgcaatggaaaaagaaaagtacaatatgagagcagtgagcctgcagattttaaggtggatgaagatggcatggtgtatgccgtgagaagctttccactctcttctgagcatgccaagttcctgatatatgcccaagacaaagagacccaggaaaagtggcaagtggcagtaaaattgagcctgaagccaaccttaactgaggagtcagtgaaggagtcagcagaagttgaagaaatagtgttcccaagacaattcagtaagcacagtggccacctacaaaggcagaagagagactgggtcatccctccaatcaacttgccagaaaactccaggggaccttttcctcaagagcttgtcaggatcaggtctgatagagataaaaacctttcactgcggtacactgtaactgggccaggagctgaccagcctccaactggtatcttcattatcaaccccatctcgggtcagctgtcggtgacaaagcccctggatcgcgagcagatagcccggtttcatttgagggcacatgcagtagatattaatggaaatcaagtggagaaccccattgacattgtcatcaatgttattgacatgaatgacaacagacctgagttcttacaccaggtttggaatgggacagttcctgagggatcaaagcctggaacatatgtgatgaccgtaacagcaattgatgctgacgatcccaatgccctcaatgggatgttgaggtacagaatcgtgtctcaggctccaagcaccccttcacccaacatgtttacaatcaacaatgagactggtgacatcatcacagtggcagctggacttgatcgagaaaaagtgcaacagtatacgttaataattcaagctacagacatggaaggaatccccacatatggcctttcaaacacagccacggccgtcatcacagtgacagatgtcaatgacaatcctccagagtttactgccatgacgttttatggtgaagttcctgagaacagggtagacatcatagtagctaatctaactgtgaccgataaggatcaaccccatacaccagcctggaacgcagtgtacagaatcagtggcggagatcctactggacggttcgccatccagaccgacccaaacagcaacgacgggttagtcaccgtggtcaaaccaatcgactttgaaacaaataggatgtttgtccttactgttgctgcagaaaatcaagtgccattagccaagggaattcagcacccgcctcagtcaactgcaaccgtgtctgttacagttattgacgtaaatgaaaacccttattttgcccccaatcctaagatcattcgccaagaagaagggcttcatgccggtaccatgttgacaacattcactgctcaggacccagatcgatatatgcagcaaaatattagatacactaaattatctgatcctgccaattggctaaaaatagatcctgtgaatggacaaataactacaattgctgttttggaccgagaatcaccaaatgtgaaaaacaatatatataatgctactttccttgcttctgacaatggaattcctcctatgagtggaacaggaacgctgcagatctatttacttgatattaatgacaatgcccctcaagtgttacctcaagaggcagagacttgcgaaactccagaccccaattcaattaatattacagcacttgattatgacattgatccaaatgctggaccatttgcttttgatcttcctttatctccagtgactattaagagaaattggaccatcactcggcttaatggtgattttgctcagcttaatttaaagataaaatttcttgaagctggtatctatgaagttcccatcataatcacagattcgggtaatcctcccaaatcaaatatttccatcctgcgcgtgaaggtttgccagtgtgactccaacggggactgcacagatgtggacaggattgtgggtgcggggcttggcaccggtgccatcattgccatcctgctctgcatcatcatcctgcttatccttgtgctgatgtttgtggtatggatgaaacgccgggataaagaacgccaggccaaacaacttttaattgatccagaagatgatgtaagagataatattttaaaatatgatgaagaaggtggaggagaagaagaccaggactatgacttgagccagctgcagcagcctgacactgtggagcctgatgccatcaagcctgtgggaatccgacgaatggatgaaagacccatccacgctgagccccagtatccggtccgatctgcagccccacaccctggagacattggggacttcattaatgagggccttaaagcggctgacaatgaccccacagctccaccatatgactccctgttagtgtttgactatgaaggcagtggctccactgctgggtccttgagctcccttaattcctcaagtagtggtggtgagcaggactatgattacctgaacgactgggggccacggttcaagaaacttgctgacatgtatggtggaggtgatgactgaacttcagggtgaacttggtttttggacaagt

SEQ ID NO: 46 - protein, 906 aa, Homo sapiens [sequence omitted]

SEQ ID NO: 47 - DNA, 2808 bp, Homo sapiens
ggaaagcacctgtgagcttggcaagtcagttcagagctccagcccgctccagcccggcccgacccgaccgcacccggcgcctgcctcgctcgggctccccggccagccatgggcccttggagccgcagcctctcgggcctgctgctgctgctgaggtctcctcttggctctcaggagcggagccctcctccctgtttgacgcgagagctacacgttcacggtgccccggcgccacctgagaagaggccgcgtctgggcagagtgaattttgaagattgcaccggtcgacaaaggacagctattttcctgacaccgattccgaaagtgggcacagatggtgtgattacagtcaaaaggcctctacggtttcataacccaacagatccatttcttggtctacgctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaaggccccatctttgtgcctcctgaaaagagagtggaagtgtccgaggactttggcgtgggccaggaaatcacatcctacactgcccaggagccagacacatttatggaacagaaaataacatatcggatttggagagacactcgcaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacattcatgatgcagaccttcctcccaatacatctcccttcacagcagaactaacacacgggcgagtgcccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgccggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatccgttcaagaagctggctgacatgtacggaggcggcgaggaccactaggggactcgagagaggcggcccagaccatgtgcagaaatgcagaaatcagcgttctggtgtttt

SEQ ID NO: 48 - protein, 878 aa, Homo sapiens [sequence omitted]

SEQ ID NO: 49 - DNA, 3171 bp, Homo sapiens
gcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacagatgaggatgatgccatctacacctacaatggggtggttgcttactccatccatagccaagaaccaaaggacccacacgacctcatgttcacaattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaacgaccatggcccagtccctgagccccgtcagatcaccatctgcaaccaaagccctgtgcgccacgtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaaaggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctaggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaa

SEQ ID NO: 50 - protein, 829 aa, Homo sapiens [sequence omitted]

SEQ ID NO: 51 - DNA, 2750 bp, Homo sapiens
gcacgatctgttcctcctgggaagatgcagaggctcatgatgctcctcgccacatcgggcgcctgcctgggcctgctggcagtggcagcagtggcagcagcaggtgctaaccctgcccaacgggacacccacagcctgctgcccacccaccggcgccaaaagagagattggatttggaaccagatgcacattgatgaagagaaaaacacctcacttccccatcatgtaggcaagatcaagtcaagcgtgagtcgcaagaatgccaagtacctgctcaaaggagaatatgtgggcaaggtcttccgggtcgatgcagagacaggagacgtgttcgccattgagaggctggaccgggagaatatctcagagtaccacctcactgctgtcattgtggacaaggacactggtgaaaacctggagactccttccagcttcaccatcaaagttcatgacgtgaacgacaactggcctgtgttcacgcatcggttgttcaatgcgtccgtgcctgagtcgtcggctgtggggacctcagtcatctctgtgacagcagtggatgcagacgaccccactgtgggagaccacgcctctgtcatgtaccaaatcctgaaggggaaagagtattttgccatcgataattctggacgtattatcacaataacgaaaagcttggaccgagagaagcaggccaggtatgagatcgtggtggaagcgcgagatgcccagggcctccggggggactcgggcacggccaccgtgctggtcactctgcaagacatcaatgacaacttccccttcttcacccagaccaagtacacatttgtcgtgcctgaagacacccgtgtgggcacctctgtgggctctctgtttgttgaggacccagatgagccccagaaccggatgaccaagtacagcatcttgcggggcgactaccaggacgctttcaccattgagacaaaccccgcccacaacgagggcatcatcaagcccatgaagcctctggattatgaatacatccagcaatacagcttcatcgtcgaggccacagaccccaccatcgacctccgatacatgagccctcccgcgggaaacagagcccaggtcattatcaacatcacagatgtggacgagccccccattttccagcagcctttctaccacttccagctgaaggaaaaccagaagaagcctctgattggcacagtgctggccatggaccctgatgcggctaggcatagcattggatactccatccgcaggaccagtgacaagggccagttcttccgagtcacaaaaaagggggacatttacaatgagaaagaactggacagagaagtctacccctggtataacctgactgtggaggccaaagaactggattccactggaacccccacaggaaaagaatccattgtgcaagtccacattgaagttttggatgagaatgacaatgccccggagtttgccaagccctaccagcccaaagtgtgtgagaacgctgtccatggccagctggtcctgcagatctccgcaatagacaaggacataacaccacgaaacgtgaagttcaaattcaccttgaatactgagaacaactttaccctcacggataatcacgataacacggccaacatcacagtcaagtatgggcagtttgaccgggagcataccaaggtccacttcctacccgtggtcatctcagacaatgggatgccaagtcgcacgggcaccagcacgctgaccgtggccgtgtgcaagtgcaacgagcagggcgagttcaccttctgcgaggatatggccgcccaggtgggcgtgagcatccaggcagtggtagccatcttactctgcatcctcaccatcacagtgatcaccctgctcatcttcctgcggcggcggctccggaagcaggcccgcgcgcacggcaagagcgtgccggagatccacgagcagctggtcacctacgacgaggagggcggcggcgagatggacaccaccagctacgatgtgtcggtgctcaactcggtgcgccgcggcggggccaagcccccgcggcccgcgctggacgcccggccttccctctatgcgcaggtgcagaagccaccgaggcacgcgcctggggcacacggagggcccggggagatggcagccatgatcgaggtgaagaaggacgaggcggaccacgacggcgacggccccccctacgacacgctgcacatctacggctacgagggctccgagtccatagccgagtccctcagctccctgggcaccgactcatccgactctgacgtggattacgacttccttaacgactggggacccaggtttaagatgctggctgagctgtacggctcggacccccgggaggagctgctgtattaggcggccgaggtcactctgggcctggggacccaaaccccctgcagcccaggccagtcagactccaggcaccacagcctccaaaaatggcagtgactccccagcccagcaccccttcctcgtgggtcccagagacctcatcagccttgggatagcaaactccaggttcctgaaatatccaggaatatatgtcagtgatgactattctcaaatgctggcaaatccaggctggtgttctgtctgggctcagacatccacataaccctgtcacccacagaccgccgtctaactcaaagacttcctctggctccccaaggctgcaaagcaaaacagactgtgtttaactgctgcagggtctttttctagggtccctg

SEQ ID NO: 52 - protein, 784 aa, Homo sapiens
MQRLMMLLATSGACLGLLAVAAVAAAGANPAQRDTHSLLPTHRRQKRDWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVFTHRLFNASVPESSAVGTSVISVTAVDADDPTVGDHASVMYQILKGKEYFAIDNSGRIITITKSLDREKQARYEIVVEARDAQGLRGDSGTATVLVTLQDINDNFPFFTQTKYTFVVPEDTRVGTSVGSLFVEDPDEPQNRMTKYSILRGDYQDAFTIETNPAHNEGIIKPMKPLDYEYIQQYSFIVEATDPTIDLRYMSPPAGNRAQVIINITDVDEPPIFQQPFYHFQLKENQKKPLIGTVLAMDPDAARHSIGYSIRRTSDKGQFFRVTKKGDIYNEKELDREVYPWYNLTVEAKELDSTGTPTGKESIVQVHIEVLDENDNAPEFAKPYQPKVCENAVHGQLVLQISAIDKDITPRNVKFKFTLNTENNFTLTDNHDNTANITVKYGQFDREHTKVHFLPVVISDNGMPSRTGTSTLTVAVCKCNEQGEFTFCEDMAAQVGVSIQAVVAILLCILTITVITLLIFLRRRLRKQARAHGKSVPEIHEQLVTYDEEGGGEMDTTSYDVSVLNSVRRGGAKPPRPALDARPSLYAQVQKPPRHAPGAHGGPGEMAAMIEVKKDEADHDGDGPPYDTLHIYGYEGSESIAESLSSLGTDSSDSDVDYDFLNDWGPRFKMLAELYGSDPREELLY

SEQ ID NO: 53 - DNA, 2721 bp, Homo sapiens
atgactgctgtccatgcaggcaacataaacttcaagtgggatcctaaaagtctagagatcaggactctggcagttgagagactgttggagcctcttgttacacaggttacaacccttgtaaacaccaatagtaaagggccctctaataagaagagaggtcgttctaagaaggcccatgttttggctgcatctgttgaacaagcaactgagaatttcttggagaagggggataaaattgcgaaggagagccagtttctcaaggaggagcttgtggctgctgtagaagatgttcgaaaacaaggtgatttgatgaaggctgctgcaggagagttcgcagatgatccctgctcttctgtgaagcgaggcaacatggttcgggcagctcgagctttgctctctgctgttacccggttgctgattttggctgacatggcagatgtctacaaattacttgttcagctgaaagttgtggaagatggtatcttgaagttgaggaatgctggcaatgaacaagacttaggaatccagtataaagccctaaaacctgaagtggataagctgaacattatggcagccaaaagacaacaggaattgaaagatgttggccatcgtgatcagatggctgcagctagaggaatcctgcagaagaacgttccgatcctctatactgcatcccaggcatgcctacagcaccctgatgtcgcagcctataaggccaacagggacctgatatacaagcagctgcagcaggcggtcacaggcatttccaatgcagcccaggccactgcctcagacgatgcctcacagcaccagggtggaggaggaggagaactggcatatgcactcaataactttgacaaacaaatcattgtggaccccttgagcttcagcgaggagcgctttaggccttccctggaggagcgtctggaaagcatcattagtggggctgccttgatggccgactcgtcctgcacgcgtgatgaccgtcgtgagcgaattgtggcagagtgtaatgctgtccgccaggccctgcaggacctgctttcggagtacatgggcaatgctggacgtaaagaaagaagtgatgcactcaattctgcaatagataaaatgaccaagaagaccagggacttgcgtagacagctccgcaaagctgtcatggaccacgtttcagattctttcctggaaaccaatgttccacttttggtattgattgaagctgcaaagaatggaaatgagaaagaagttaaggagtatgcccaagttttccgtgaacatgccaacaaattgattgaggttgccaacttggcctgttccatctcaaataatgaagaaggtgtaaagcttgttcgaatgtctgcaagccagttagaagccctctgtcctcaggttattaatgctgcactggctttagcagcaaaaccacagagtaaactggcccaagagaacatggatctttttaaagaacaatgggaaaaacaagtccgtgttctcacagatgctgtcgatgacattacttccattgatgacttcttggctgtctcagagaatcacattttggaagatgtgaacaaatgtgtcattgctctccaagagaaggatgtggatggcctggaccgcacagctggtgcaattcgaggccgggcagcccgggtcattcacgtagtcacctcagagatggacaactatgagccaggagtctacacagagaaggttctggaagccactaagctgctctccaacacagtcatgccacgttttactgagcaagtagaagcagccgtggaagccctcagctcggaccctgcccagcccatggatgagaatgagtttatcgatgcttcccgcctggtatatgatggcatccgggacatcaggaaagcagtgctgatgataaggacccctgaggagttggatgactctgactttgagacagaagattttgatgtcagaagcaggacgagcgtccagacagaagacgatcagctgatagctggccagagtgcccgggcgatcatggctcagcttccccaggagcaaaaagcgaagattgcggaacaggtggccagcttccaggaagaaaagagcaagctggatgctgaagtgtccaaatgggacgacagtggcaatgacatcattgtgctggccaagcagatgtgcatgattatgatggagatgacagactttacccgaggtaaaggaccactcaaaaatacatcggatgtcatcagtgctgccaagaaaattgctgaggcaggatccaggatggacaagcttggccgcaccattgcagaccattgccccgactcggcttgcaagcaggacctgctggcctacctgcaacgcatcgccctctactgccaccagctgaacatctgcagcaaggtcaaggccgaggtgcagaatctcggcggggagcttgttgtctctggggtggacagcgccatgtccctgatccaggcagccaagaacttgatgaatgctgtggtgcagacagtgaaggcatcctacgtcgcctctaccaaataccaaaagtcacagggtatggcttccctcaaccttcctgctgtgtcatggaagatgaaggcaccagagaaaaagccattggtgaagagagagaaacaggatgagacacagaccaagattaaacgggcatctcagaagaagcacgtgaacccggtgcaggccctcagcgagttcaaagctatggacagcatctaa

SEQ ID NO: 54 - protein, 906 aa, Homo sapiens
MTAVHAGNINFKWDPKSLEIRTLAVERLLEPLVTQVTTLVNTNSKGPSNKKRGRSKKAHVLAASVEQATENFLEKGDKIAKESQFLKEELVAAVEDVRKQGDLMKAAAGEFADDPCSSVKRGNMVRAARALLSAVTRLLILADMADVYKLLVQLKVVEDGILKLRNAGNEQDLGIQYKALKPEVDKLNIMAAKRQQELKDVGHRDQMAAARGILQKNVPILYTASQACLQHPDVAAYKANRDLIYKQLQQAVTGISNAAQATASDDASQHQGGGGGELAYALNNFDKQIIVDPLSFSEERFRPSLEERLESIISGAALMADSSCTRDDRRERIVAECNAVRQALQDLLSEYMGNAGRKERSDALNSAIDKMTKKTRDLRRQLRKAVMDHVSDSFLETNVPLLVLIEAAKNGNEKEVKEYAQVFREHANKLIEVANLACSISNNEEGVKLVRMSASQLEALCPQVINAALALAAKPQSKLAQENMDLFKEQWEKQVRVLTDAVDDITSIDDFLAVSENHILEDVNKCVIALQEKDVDGLDRTAGAIRGRAARVIHVVTSEMDNYEPGVYTEKVLEATKLLSNTVMPRFTEQVEAAVEALSSDPAQPMDENEFIDASRLVYDGIRDIRKAVLMIRTPEELDDSDFETEDFDVRSRTSVQTEDDQLIAGQSARAIMAQLPQEQKAKIAEQVASFQEEKSKLDAEVSKWDDSGNDIIVLAKQMCMIMMEMTDFTRGKGPLKNTSDVISAAKKIAEAGSRMDKLGRTIADHCPDSACKQDLLAYLQRIALYCHQLNICSKVKAEVQNLGGELVVSGVDSAMSLIQAAKNLMNAVVQTVKASYVASTKYQKSQGMASLNLPAVSWKMKAPEKKPLVKREKQDETQTKIKRASQKKHVNPVQALSEFKAMDSI

SEQ ID NO: 55 - DNA, 2712 bp, Xenopus laevis
atgactctcaatacaggaaacataaatttcaagtgggatccaaaaagcttggaaataagaacactagccgttgagagacttcttgagcctttagtatctcaggtgactactttggtgaatactagcaataaaggaccatccaataaaaagaaagggcgttcaaagaaagctcatgttttggctgcatcagtggagcaagcaacccaaaattttttggataaaggagacaaaatcgcaaaagacagccagtttcttaaagaggaactcattgcagcagttgaagatgttcggaaacagggtgaacagatgagaagcgcctctggagagtttgcggatgatccttgctcttctgttaaacgtggaaacatggtccgtgctgcacgtgcattgttgtctgctgtcacaaggctgctgattttggctgacatggcagatgtgtacaggttactggttcagctgaaagtggttgaagaaggtattctgaaattaagaaatgctggaactgaacaagatttaggaatacaatataaggctttgagagctgaagtggataaattaaatgtcatgacggcaaagagacaacaggaattaaaggatattggccacagagatcagatggctgcagctcgtggtattcttcagaagagcattcctattctttacactgcttcacaggcatgtctgcagcatcccgatgtagctgcatacaaagctaatagggacttggtatataaacaacttcaacaagctgtcagtggcatttcaaatgcagcccaagctacatcgtctgaagaaagtgctcaacaacaaggaggtggagaacttgctgttgccctaaataattttgataaacaaatcattgtggatcctcttggctttagtgaagaaagatttagaccttcactagaggagcgtttagaaagcatcattagtggagcagccctaatggcagattcttcttgcacacgagatgatcgccgggaacgtattgttgctgaatgcaactctgtgagacaagctctacaagatctcctttctgaatacatgggaaatactggccgcaaggaacgtggtgatgctctaaattcagcaatcgacaaaatgacaaggaaaaccagagacttgcgtagacagttgagaaaggctgttatggaccatgtttctgactctttcttggagaccaatgtaccactacttgtgttgattgaggctgcaaagaatggaaatgaaaaagaagttaaagagtatgcacaagtatttcgtgaacatgccaataaactgattgaggtagccaatttggcctgttccatatccaacaacgaagaaggtgtaaaactagtccgtatatctgctggacagcttgaatctctttgcccacaggttataaatgcagctttggccttggcagctaagccgaatagtaaaatggcacaggaaaatatggatctttacaaagagcaatgggaaagacaagttcgagtcctaacagatgctgttgatgatattacatcaattgatgacttcttggccgtttcagaaaatcacattttggaggatgtcaataaatgtgtcatagctcttcaggagagagatgttgatggcttggatcgtacagctggagctattcgtggacgtgcagcaagagtcatacatgttgtcacctctgaaatggataactatgaacctggaatttacacagaaaaggttctggaagctactaaattgctgacaaatacagttatgccacgtttcacggagcaagttgaagctgcagttgaggcccttagtggagacaccaatcagaccatggatgaaaatgaatttattgatgcttctcgactagtatatgatggtgtacgggacatccgaaaagctgtgctgatgatcagaactccagaagaactagatgattctgattttgaaactgaagactttgatgtgagaagcagaacaagtgtccagacagaggatgaccagcttattgctggacagagtgcaagagcaatcatggctcagcttcctcaggaacaaaaggcaaaaattgcagaacaagtggctagcttccaggaagaaaaaagcaaactggatgctgaggtgtcgaaatgggatgacaatggaaacgacctcattgttttggctaaacagatgtgtatgataatgatggaaatgactgactttactagagggaaggggccactgaaaaacacttcagatgtaattagcgcagctaagaaaattgcagaagcggggtcacgaatggataaattaggacgtactattgcagatcactgccctgattcaacttgtaagcaagatcttttagcatatcttcagagaattgccttgtactgccatcagttaaatatatgtagtaaagtaaaggcagaagttcagaatcttggcggcgagcttgttgtgtctggggttgacagtgccatgtccctgatacaagccgcgaaaaatctgatgaatgccgttgttcagaccgtaaaagcctcctatgtagcttctacgaaataccagaagtcacaaggaatggcatctctgaatctgcctgcagtgtcatggaaaatgaaagctccagaaaagaaaccacttgttaagagagagaagcaagatgagacccaaactaagataaagcgtgcttctcagaaaaaacatgtcaacccagtgcaggctctaagtgaatttaaagcaatggaaagcatttaa

SEQ ID NO: 56 - protein, 903 aa, Xenopus laevis
MTLNTGNINFKWDPKSLEIRTLAVERLLEPLVSQVTTLVNTSNKGPSNKKKGRSKKAHVLAASVEQATQNFLDKGDKIAKDSQFLKEELIAAVEDVRKQGEQMRSASGEFADDPCSSVKRGNMVRAARALLSAVTRLLILADMADVYRLLVQLKVVEEGILKLRNAGTEQDLGIQYKALRAEVDKLNVMTAKRQQELKDIGHRDQMAAARGILQKSIPILYTASQACLQHPDVAAYKANRDLVYKQLQQAVSGISNAAQATSSEESAQQQGGGELAVALNNFDKQIIVDPLGFSEERFRPSLEERLESIISGAALMADSSCTRDDRRERIVAECNSVRQALQDLLSEYMGNTGRKERGDALNSAIDKMTRKTRDLRRQLRKAVMDHVSDSFLETNVPLLVLIEAAKNGNEKEVKEYAQVFREHANKLIEVANLACSISNNEEGVKLVRISAGQLESLCPQVINAALALAAKPNSKMAQENMDLYKEQWERQVRVLTDAVDDITSIDDFLAVSENHILEDVNKCVIALQERDVDGLDRTAGAIRGRAARVIHVVTSEMDNYEPGIYTEKVLEATKLLTNTVMPRFTEQVEAAVEALSGDTNQTMDENEFIDASRLVYDGVRDIRKAVLMIRTPEELDDSDFETEDFDVRSRTSVQTEDDQLIAGQSARAIMAQLPQEQKAKIAEQVASFQEEKSKLDAEVSKWDDNGNDLIVLAKQMCMIMMEMTDFTRGKGPLKNTSDVISAAKKIAEAGSRMDKLGRTIADHCPDSTCKQDLLAYLQRIALYCHQLNICSKVKAEVQNLGGELVVSGVDSAMSLIQAAKNLMNAVVQTVKASYVASTKYQKSQGMASLNLPAVSWKMKAPEKKPLVKREKQDETQTKIKRASQKKHVNPVQALSEFKAMESI

SEQ ID NO: 57 - DNA, 2718 bp, Mus musculus
atgacttcggcaacttcacctattattttaaaatgggatcccaaaagtttggaaatccggacactcacagtggaaagactattggagccacttgtgacacaggtgacaacacttgtcaacacaagcaacaaaggtccgtctggtaaaaagaaagggaggtcaaagaaagcccatgtgctggcagcatctgtagaacaagctactcagaacttcctggaaaagggtgaacagatcgctaaggagagccaagacctcaaagaagagttagtggctgctgtagaggatgtgcggaagcaaggtgagacaaagcggattgcctcctcagagtttgcagatgacccttgctcttctgtcaagcgtggcaccatggtgcgtgcagcacgggctctgctatcggctgtgacacgcttgctcatcctggccgacatggcagatgtcatgaggcttttatcgcatctgaaaattgtcgaggaggccttggaagcagtcaaaaatgccacaaatgaacaagaccttgcaaaccgatttaaagagtttgggaaagagatggtgaaactgaactatgtagcagcaagacggcagcaggagctcaaggaccctcactgtagggatgagatggctgcagcccgtggagccctgaagaagaatgccaccatgctgtacacagcctcccaagccttcctccggcatccagatgttgctgctacaagagccaaccgagattatgtatttaaacaagtccaagaggccatagctggcatctccagtgctgctcaggccacctcccccaccgatgaagccaaaggccacacaggcatcggcgagctggctgcagccctgaatgagtttgataataagatcatcctggaccccatgacattcagcgaggccaggttccgaccatccctggaggagagactggagagcatcatcagtggggctgctctcatggcagattcctcctgcacacgtgatgaccgccgtgagcgtatggtggccgagtgcaatgcagttcgacaggcactccaggacctgctaagcgaggacatgaataacactggaaggaaagagaaaggagaccctctcaacattgcgattgacaagatgaccaagaaaacaagagatctgaggagacagcttcggaaagctgtgatggatcacatctcagattctttcttggaaaccaatgtccccttgctggttctcattgaggctgcgaagagcgggaatgagaaggaggtgaaggaatacgcccaagttttccgtgaacatgccaacaagctggtggaggttgccaatttggcttgttccatctccaacaatgaggaaggggtgaaattagtcagaatggcagccacccagattgacagcctgtgtccccaagtcattaatgctgccctcacactggctgctcggccacagagtaaagttgctcaggacaacatggatgtcttcaaagaccagtgggaaaagcaagtccgtgtgctcactgaggcagtggatgacatcacctctgtggatgacttcctctctgtctcagaaaaccatatcttggaggatgtgaacaaatgtgtgattgccctgcaagagggagatgtggacacactggatcgcacagctggggccatacggggccgggcagcccgggtcattcacatcatcaatgcagagatggagaactatgaagctggggtctatacagagaaagtgctggaagccacaaaattgctttcagaaacagtgatgccacgctttgctgaacaagttgaggtggccatcgaagccctgagcgccaatgtccctcagccattcgaggagaacgagttcatcgatgcctcgcgcctggtgtattacggtgttcgggacatcagaaaggctgtgctgatgatcaggactccagaagagctagaagatgattccgactttgagcaagaggattatgatgtccgcagtcggacaagtgtccagacagaggaccgacagctcattgctggacagagtgcacgggccatcatggcgcaactaccacaggaggagaaagcaaaaatagctgaacaggtggagattttccaccaagaaaaaagcaagctggatgctgaagtggccaagtgggatgacagcggcaatgacatcattgtgctggccaagcagatgtgtatgatcatgatggagatgacagacttcacaagaggcaaaggcccactgaaaaatacatctgatgtcattaatgctgccaagaagattgcagaagcaggctctcgaatggacaaattagcgcgcgctgtggctgatcagtgtcctgattcagcatgtaagcaggatttattagcctaccttcagcggattgctttgtactgccatcagcttaacatctgcagcaaagtgaaggccgaggttcagaacctaggaggagagctcattgtgtcagggctggacagttctacatcactcatccaggcagccaaaaacctgatgaatgctgttgtcctcacggtgaaagcgtcttatgtagcctcaactaaataccagaaggtctatggaacagcagctgtcaactctccagttgtgtcttggaagatgaaggctcctgaaaagaagccccttgtgaagagagaaaagcctgaagaattccagacaagagttagacgggggtctcaaaagaaacacatttcacctgtgcaggctttaagcgaattcaaggcaatggattccttctag

SEQ ID NO: 58 - protein, 905 aa, Mus musculus
MTSATSPIILKWDPKSLEIRTLTVERLLEPLVTQVTTLVNTSNKGPSGKKKGRSKKAHVLAASVEQATQNFLEKGEQIAKESQDLKEELVAAVEDVRKQGETKRIASSEFADDPCSSVKRGTMVRAARALLSAVTRLLILADMADVMRLLSHLKIVEEALEAVKNATNEQDLANRFKEFGKEMVKLNYVAARRQQELKDPHCRDEMAAARGALKKNATMLYTASQAFLRHPDVAATRANRDYVFKQVQEAIAGISSAAQATSPTDEAKGHTGIGELAAALNEFDNKIILDPMTFSEARFRPSLEERLESIISGAALMADSSCTRDDRRERMVAECNAVRQALQDLLSEDMNNTGRKEKGDPLNIAIDKMTKKTRDLRRQLRKAVMDHISDSFLETNVPLLVLIEAAKSGNEKEVKEYAQVFREHANKLVEVANLACSISNNEEGVKLVRMAATQIDSLCPQVINAALTLAARPQSKVAQDNMDVFKDQWEKQVRVLTEAVDDITSVDDFLSVSENHILEDVNKCVIALQEGDVDTLDRTAGAIRGRAARVIHIINAEMENYEAGVYTEKVLEATKLLSETVMPRFAEQVEVAIEALSANVPQPFEENEFIDASRLVYYGVRDIRKAVLMIRTPEELEDDSDFEQEDYDVRSRTSVQTEDRQLIAGQSARAIMAQLPQEEKAKIAEQVEIFHQEKSKLDAEVAKWDDSGNDIIVLAKQMCMIMMEMTDFTRGKGPLKNTSDVINAAKKIAEAGSRMDKLARAVADQCPDSACKQDLLAYLQRIALYCHQLNICSKVKAEVQNLGGELIVSGLDSSTSLIQAAKNLMNAVVLTVKASYVASTKYQKVYGTAAVNSPVVSWKMKAPEKKPLVKREKPEEFQTRVRRGSQKKHISPVQALSEFKAMDSF

SEQ ID NO: 59 - DNA, 2808 bp, Drosophila melanogaster
atgctgcagccagctctcccgctccactgtatatatccgtcggttaaagaatttatgttaaaacctgataaaatgggcacgttaaccgatttcggacagatagctttgaaatgggatcccaaaaatttggaaattcgcacaatgtcagttgaaaaaacacttgaaccccttgtattacaagtaactactcttgtaaataccaagggcccaagcaaaaagaaaaaaggaaaatcaaagcgggccagcgcattagttgcagctgttgaaaaagctacagaaaattttattcaaaaaggtgaacagatcgcttacgagaacccagacattacacaagaaatgttaacagctgtggatgaagtaaaaaaaactggagatgctatgagcattgcagccagagaattttctgaagatccgtgcagttccctgaagagaggaaatatggtgcgcgcagctaggaatctgttgtcagctgtaacccgcttgctgattttagctgatatggttgatgtacatttgctcttaaaatcactccacattgtcgaagatgatctaaacaaactcaaaaacgcttcgagtcaggacgagcttatggataatatgaggcaattcggacgcaatgcaggagaacttataaaacaggcagccaaacgtcagcaagaacttaaggatccccaattaagggacgatttagcagctgctcgggcgatgcttaaaaaacattcaactatgctgttaactgcatcaaaagtatacgttcgtcatccggaactagatctagcaaaagtaaatcgcgatttcattctaaaacaagtttgcgatgctgtaaatactattagcgatgttgcccaaggaaagtcatcccaaccgacagatatatacagtggagcgggagagctggctgcagcattagacgactttgacgaaggaattgttatggatcccatgacctacagcgaaaagcgttcacgtcaattgctcgaagagcgtctggaaagtattattagtgcagctgcattgatggcggatgcagattgtactcgagacgagagacgagagagaattgtggccgaatgcaatgctgtgcgacaggctttacaggatttgttatctgaatatatgtcaaacatgagtcaaaaagataacagcccaggactctctcgagcgattgatcaaatgtgtcgaaagactcgcgacctaagaaggcaattgcgaaaggctgttgtggatcatgtttcggactcttttttggagactacaactcctttgctagatttaattgaagctgcaaaatcgggtaatgaaaaaaaagttcgggaaaagtccgaaatattcaccaagcacgctgaaaaactagttgaagtagcaaatttagtatgtagcatgtcaaacaatgaagatggtgttaaaatggttcgatatgctgctgctcaaattgaaagtctttgtccgcaagtaataaatgcagcatcgatattgactgttagaccgaattcaaaagtagctcaagaaaatatgactacttatcgacaagcctgggaggtgcaagttcgtattttaaccgaggcagtggatgatattacaacaattgacgactttttagcagtatctgagaatcacattcttgaagatgtaaacaaatgtgtaatggctttacaagttggtgatgccagggatttgcgtgcaactgctggtgctattcaaggtcgatcatcacgagtttgtaatgttgttgaagctgaaatggataattatgaaccatgtatttacaccaaacgagtcttagaagcagtcaaagttctacgagatcaagttatgatgaaatttgaccaacgtgtaggagcagccgttggagccctttcaaataactccaataaggatgttgacgaaaatgacttcattgatgcttctcgtttggtttacgacggagttcgtgaaattcgaagagctgttttaatgaatcgaagctcggaagaccttgatacagatactgaatttgagccagttgaagacttaaccttggaaactcgaagtcgatcaagtgctcataccggcgatcaaaccgttgacgaatatcccgatataagtggcatatgtacagctcgagaagcaatgcgaaaaatgacggaagaagacaaacaaaaaattgctcagcaagtggagttattccgtagggaaaaactaacttttgattcagaagttgctaaatgggatgacactggaaatgacattatttttctggccaaacatatgtgcatgattatgatggaaatgacggattttaccagaggacgaggacctttaaaaactactatggatgttattaacgcagctaaaaaaatttcggaagctggtacaaagctggataaactaaccagggaaatagcagagcaatgcccagaaagcagcacgaaaaaggaccttttagcgtatttgcaacgcattgccctgtattgtcatcaaatccaaataacttcaaaagtaaaagcagacgttcaaaatataagcggcgaacttatagtttctgggctggacagcgctacatcgttaattcaagctgctaaaaatttaatgaatgccgttgtattaacggtaaagtactcatatgtggcatcaacaaaatataccaggcaaggaacagtgtcttctccaattgttgtatggaagatgaaagcaccagaaaaaaaaccattggtcaggcctgaaaaaccagaagaagtgcgggccaaagttcgcaagggatctcaaaaaaaggttcaaaaccctatacatgcattatctgaattccagagtcctgctgacgctgtttaa

SEQ ID NO: 60 - protein, 935 aa, Drosophila melanogaster
MLQPALPLHCIYPSVKEFMLKPDKMGTLTDFGQIALKWDPKNLEIRTMSVEKTLEPLVLQVTTLVNTKGPSKKKKGKSKRASALVAAVEKATENFIQKGEQIAYENPDITQEMLTAVDEVKKTGDAMSIAAREFSEDPCSSLKRGNMVRAARNLLSAVTRLLILADMVDVHLLLKSLHIVEDDLNKLKNASSQDELMDNMRQFGRNAGELIKQAAKRQQELKDPQLRDDLAAARAMLKKHSTMLLTASKVYVRHPELDLAKVNRDFILKQVCDAVNTISDVAQGKSSQPTDIYSGAGELAAALDDFDEGIVMDPMTYSEKRSRQLLEERLESIISAAALMADADCTRDERRERIVAECNAVRQALQDLLSEYMSNMSQKDNSPGLSRAIDQMCRKTRDLRRQLRKAVVDHVSDSFLETTTPLLDLIEAAKSGNEKKVREKSEIFTKHAEKLVEVANLVCSMSNNEDGVKMVRYAAAQIESLCPQVINAASILTVRPNSKVAQENMTTYRQAWEVQVRILTEAVDDITTIDDFLAVSENHILEDVNKCVMALQVGDARDLRATAGAIQGRSSRVCNVVEAEMDNYEPCIYTKRVLEAVKVLRDQVMMKFDQRVGAAVGALSNNSNKDVDENDFIDASRLVYDGVREIRRAVLMNRSSEDLDTDTEFEPVEDLTLETRSRSSAHTGDQTVDEYPDISGICTAREAMRKMTEEDKQKIAQQVELFRREKLTFDSEVAKWDDTGNDIIFLAKHMCMIMMEMTDFTRGRGPLKTTMDVINAAKKISEAGTKLDKLTREIAEQCPESSTKKDLLAYLQRIALYCHQIQITSKVKADVQNISGELIVSGLDSATSLIQAAKNLMNAVVLTVKYSYVASTKYTRQGTVSSPIVVWKMKAPEKKPLVRPEKPEEVRAKVRKGSQKKVQNPIHALSEFQSPADAV